(19) United States
(12) Patent Application Publication
(10) Pub. No.: US 2025/0342968 A1
(43) Pub. Date: Nov. 6, 2025

(54) IDENTIFICATION OF FEATURES FOR PREDICTING A PARTICULAR CHARACTERISTIC

(71) Applicant: Hoffmann-La Roche Inc., Little Falls, NJ (US)

(72) Inventors: Richard Alexander BARBIERI, Houston, TX (US); James Jinsong CAI, White Plains, NY (US); Jehad CHARO, Wettswil am Albis (CH); Vitalay FOMIN, Little Falls, NJ (US); Kenly HILLER-BITTROLFF, Marion, MA (US); WeiQing Venus SO, Verona, NJ (US)

(21) Appl. No.: 18/857,176

(22) PCT Filed: Apr. 25, 2023
(86) PCT No.: PCT/EP2023/060854

§ 371 Date: Oct. 15, 2024

(30) Foreign Application Priority Data
Apr. 26, 2022 (EP) 22170145.1

(51) Int. Cl. G16H 50/30; G16B 20/00; G16B 40/20; G16H 10/60

(52) U.S. Cl. CPC G16H 50/30; G16B 20/00; G16B 40/20; G16H 10/60

(57) ABSTRACT

A computer-implemented method of determining one or more sets of features to predict the presence of a particular phenotypic characteristic comprises: (a) receiving patient data comprising, for each of a plurality of patients: a feature profile comprising a respective feature status for each of a plurality of features for that patient; and an indication of whether that patient expresses the particular phenotypic characteristic; (b) using a genetic algorithm to generate a plurality of generations of individuals, wherein each individual comprises a subset of the predetermined plurality of features, each generation of individuals generated based, at least in part, on a plurality of fitness scores, each fitness score corresponding to a respective individual in the previous generation, and parameterizing a predictive accuracy of the set of features, each fitness score being calculated based at least in part on the patient data; (c) repeating step (b) until it has been performed N times; (d) from the plurality of individuals generated in steps (b) and (c), selecting a subset of the individuals based on their fitness scores; (e) clustering the selected subset of individuals to generate a plurality of clusters of individuals, based on the similarity of their respective subsets of features; (f) from each cluster, identifying a respective characteristic feature set based on the frequency with which features appear in individuals in that cluster.

### CROSS-REFERENCE TO RELATED APPLICATIONS

This application is a U.S. national stage application under 35 U.S.C. § 371 of International Application No. PCT/EP2023/060854, filed internationally on Apr. 25, 2023, which claims priority to European Patent Application No. 22170145.1, filed on Apr. 26, 2022, the contents of each of which are herein incorporated by reference for all purposes.

### TECHNICAL FIELD OF THE INVENTION

The present invention relates to computer-implemented methods of determining sets of features which may provide useful predictors of whether a patient is likely to display a particular phenotypic characteristic.

### BACKGROUND TO THE INVENTION

More so than ever, artificial intelligence techniques are being applied to medicine, for example in diagnosis, medical image analysis, and for tracking the status and/or progression of diseased, among many other applications. One particularly important facet of artificial intelligence, which is often applied in medical contexts is algorithms which are trained using machine-learning. Such algorithms are able to detect patterns and trends in data which may not be self-evident from human review of the data. In order to generate, train, and ultimate put to use these algorithms, it is necessary to determine which features are best correlated to the desired output. For example, it may be desirable to determine which measurements to take in order best to predict a disease status. Evidently, there are enormous of physiological and genetic features which may in some way linked to a phenotypic expression of a particular condition, or the like. Crucially, the link between the physiological or genetic feature and the phenotypic expression may not be well-established. As a result, it is often very challenging to determine a set of features which form useful predictors of a particular phenotype. This challenge is compounded by the fact that the data must be taken from real-life patients: it is not possible to control which cocktail of physiological/genetic features each patient displays in order to systematically test which features are useful predictors. The present invention aims to address these issues.

### SUMMARY OF THE INVENTION

At a high-level, the present invention provides a method of selecting features form useful predictors of a particular condition, or similar. At the heart of the invention is the repeated application of a genetic algorithm in order to generate large populations of “individuals” (which correspond to example feature profiles, and not real-life individuals), and to cluster the results in order to extract useful sets of features. It will be shown later in this application that, using these techniques, it is possible to obtain sets of features which prove to be reliable predictors in the context of prediction of CPI resistance. However, it is clear that the methods of the present invention are generally applicable to prediction of resistance to other treatments such as targeted therapies, monoclonal antibody treatment, immunotherapy, hormone therapy and chemotherapies. It is further clear that the methods of the present invention are generally applicable to prediction of other binary phenotypes and to other phenomena, medical or otherwise.

Specifically, a first aspect of the present invention provides a computer-implemented method of determining one or more sets of features to predict the presence of a particular phenotypic characteristic, the computer-implemented method comprising: (a) receiving patient data comprising, for each of a plurality of patients: a feature profile comprising a respective feature status for each of a plurality of features for that patient; and an indication of whether that patient expresses the particular phenotypic characteristic; (b) using a genetic algorithm to generate a plurality of generations of individuals, wherein each individual comprises a subset of the predetermined plurality of features, each generation of individuals generated based, at least in part, on a plurality of fitness scores, each fitness score corresponding to a respective individual in the previous generation, and parameterizing a predictive accuracy of the set of features, each fitness score being calculated based at least in part on the patient data; (c) repeating step (b) until it has been performed N times; (d) from the plurality of individuals generated in steps (b) and (c), selecting a subset of the individuals based on their fitness scores; (e) clustering selected subset of individuals to generate a plurality of clusters of individuals, based on the similarity of their respective subsets of features; (f) from each cluster, identifying a respective characteristic feature set based on the frequency with which features appear in individuals in that cluster. In some cases, it may be preferable that the plurality of clusters comprises N or more clusters.

In the context of a genetic algorithm, the term “individual” does not refer to an actual patient, or have any correspondence to a real person. Rather, the term is used to refer simply to a set of features, or an identifier of a set of features. “Phenotypic characteristic” is used to refer to any physiological characteristic that may be expressed by a patient.

We now set out various optional features of the invention.

The phenotypic characteristic may be a binary characteristic. That is, the phenotypic characteristic may be one of two possible characteristics (e.g., “resistant” and “not resistant”).

The phenotypic characteristic may be a treatment response characteristic, which may indicate resistance to a treatment. The treatment response characteristic may be a binary characteristic (e.g., “resistant” or “not resistant”). The phenotypic characteristic may be resistance to a cancer treatment.

The treatment may be a treatment that has a specific gene or protein target, e.g., certain cancer treatments. For example, the treatment may be a treatment with a defined molecular mechanism that has a specific gene or protein target, e.g., certain cancer treatments. Such phenotypic characteristics may be predictable using a binary genetic algorithm (i.e., a genetic algorithm for which the input data is binary data), which may receive input data indicating whether a gene is mutated or not, for example.

The treatment may be a cancer treatment. The computer-implemented method may be more effective than other methods for predicting cancer treatment response, because the computer-implemented method may efficiently find multiple genetic features (which may include e.g., the most predictive genes or mutations, as will be described in further detail below) that contribute to the treatment response or treatment resistance, and often multiple mutations are involved in cancers and its treatment response. The treatment may be CPI, targeted therapy (e.g. tyrosine kinase inhibitors (TKI) like imatinib, BRAF inhibitors like vemurafenib, angiogenesis inhibitors like bevacizumab), monoclonal antibodies (e.g. trastuzumab (Herceptin)), immunotherapy (e.g. checkpoint inhibitors like anti-PD1, anti-PD-L1; cytokines like interferon-alpha, interleukin-2), hormone therapy (e.g. aromatase inhibitors, selective estrogen receptor modulators (SERMs) like tamoxifen, anti-androgens) and/or chemotherapy (e.g. topoisomerase inhibitors such as irinotecan). Therefore, the phenotypic characteristic may indicate resistance to CPI, targeted therapy, monoclonal antibodies, immunotherapy, hormone therapy, and/or chemotherapy.

Step (d), in which a subset of individuals is selected based on their fitness scores, may comprise determining a predetermined number of individuals having the highest fitness scores, or a predetermined proportion of the total number of individuals having the highest fitness score, e.g. the top 10%. Alternatively, this may comprise determining a subset of the individuals whose fitness scores are in a top predetermined percentile. This may also comprise determining a subset of individuals whose fitness scores exceed a predetermined threshold). This provides a simple and reliable way of selecting a subset from what is likely to be a very large number of generated individuals. In order to achieve this, step (d) may comprise ranking all of the individuals generated using the genetic algorithm by their fitness scores, and selecting the relevant subset of individuals (i.e. predetermined number of highest-ranking individuals, a predetermined highest-ranking proportion of individuals, a subset of individuals whose fitness scores are in a top predetermined percentile, or a subset of individuals whose fitness scores exceed a predetermined threshold).

Step (f), in which a characteristic feature set is identified in each cluster, may comprise: for each cluster of individuals, identifying the one or more features which occur in more than a threshold proportion of individuals within that cluster, those features forming the respective characteristic feature set for that cluster. The threshold population may be 10% to 90%, 20% to 80%, 30% to 70%, but is preferably 40% to 60%, and most preferably about 508. This enables a balance between including only those features which appear particularly prevalent in high-fitness individuals, while ensuring that there are sufficiently many features to form a useful set of predictors. Then, step (f) may further comprise selecting one or more of the characteristic feature sets of the respective plurality of clusters as the one or more features sets to predict the presence of the particular phenotypic characteristic.

In an alternative approach, step (f) may comprise, for each cluster of individuals, identifying a set of X features in the most individuals in the cluster, those features forming the respective feature set for the cluster. The value of X may range from 40 to 180. In other words, in this alternative approach, the size of the feature set is fixed, and the X most common features in the cluster are selected. This may be achieved by ranking the features by the number of individuals within the cluster displaying that feature, and selecting the top X features. Then, as above, step (f) may further comprise selecting one or more of the characteristic feature sets of the respective plurality clusters as the one or more feature sets to predict the presence of the particular phenotypic characteristic.

Step (e) requires clustering of individuals generated using the genetic algorithm. In preferred cases, clustering the individuals comprises applying a k-means clustering algorithm on the selected individuals of the highest-ranking individuals. Other algorithms may also be used, for example UMAP or tSNE. As discussed above, it is preferable that the plurality of clusters comprises at least N clusters. In preferred cases, the plurality of clusters may comprise N+2 clusters. N is preferably no less than 10.

We now discuss in more detail how the final selection of a feature set takes place. The fitness scores are calculated based on the patient data, which means that the process is inevitably biased towards a feature set which accurately represents the patient data used to calculate the fitness scores. This is analogous e.g. to overfitting when training a machine-learning algorithm. In order to identify a set of features which accurately reflect the true dependence between the features and the phenotypic characteristic, it is therefore desirable to rely on previously unused data. Accordingly, the patient data may comprise a first subset of patient data and a second subset of patient data. Then, the fitness score is preferably calculated at least in part on the first subset of patient data, and not on the second subset of patient data. Then, step (f) may further comprise, for each identified characteristic feature set: calculating a fitness score parameterizing the predictive accuracy of the characteristic feature set based at least in part on the second subset of patient data. Preferably, the calculation is not based on the first subset of patient data. In this way, a metric indicative of the ability of a given feature set to predict the presence or absence of the phenotypic characteristic may be calculated based on data which was not used to generate the set of features in the first place, providing a more reliable selection method. Afterwards, step (f) may comprise selecting the one or more characteristic feature sets having the highest associated fitness score as the one or more feature sets which best predict the presence or absence of the particular phenotypic characteristic.

Alternatively, the step of selecting may comprise training a respective analytical model on each of the plurality of characteristic feature sets, and calculating a score representative of the predictive power of the analytical model; and selecting the characteristic feature set which yields the highest predictive power as the one or more features which best predict the presence of the particular phenotypic characteristic. The analytical model may be a machine-learning model, such as a binary or multi-class classification model. The binary classification model may be a naïve Bayes model, which may in turn comprise a Bernoulli prior.

A naïve Bayes model may be a probabilistic classifier. A naïve Bayes model may determine the probability of a certain class (a certain phenotypic characteristic in the present case) given a set of variables (a set of features in the present case). A naïve Bayes model may determine the probability of the certain class given the set of variables using Bayes' theorem. A naïve Bayes model may assume that each variable in the set of variables is independent of the other variables in the set of variables.

A naïve Bayes model may be a linear classifier.

A naïve Bayes model which comprises a Bernoulli prior may enable the interpretability of the characteristic feature set by allowing the relative importance of each type of feature to the phenotypic characteristic to be quantified, and/or by allowing each feature to be associated with the phenotypic characteristic which it predicts.

A naïve Bayes model may therefore be used in the prediction of a binary phenotypic characteristic, such as the treatment response characteristics discussed above.

Other linear classifiers may be used as alternatives to a naïve Bayes model. For example, a logistic regression classifier may be used.

The score representative of the predictive power may be a cross-validation accuracy score of the naïve Bayes model trained on the respective characteristic feature sets, on a test set which comprises a portion of the patient data on which the model has not been trained.

Optional features of the genetic algorithm are now set out.

In the context of the present invention, a “genetic algorithm” is a heuristic or metaheuristic which is inspired by the process of natural selection that belongs to the larger class of evolutionary algorithms. Genetic algorithms rely on the generation of many generations of “individuals” based on feature profiles (which in the context of computer-implemented methods which are configured to identify genetic feature sets, may be referred to herein as “genetic feature profiles” or “mutation profiles”), and by utilising biologically-inspired processes such as mutation, crossover, and selection.

The genetic algorithm may comprise the steps of: (i) generating a plurality of first generation G individuals, and for each first generation individual, calculating a fitness score; (ii) generating a plurality of second generation G₂ individuals, the subset of features of each respective second generation individual being based on the subset of features of at least one first generation individual; (iii) for each second generation individual, calculating a fitness score; and (iv) iteratively repeating steps (b) and (c) a plurality of times to generate subsequent generations Gᵢ of individuals, the subset of features of each respective individual in subsequent generations of individuals being generated based on the subset of features of at least one individual in the previous generation Gᵢ₋₁ of individuals. At a high-level, the genetic algorithm thus ensures that characteristics of individuals with higher fitness scores are carried on throughout subsequent generations, analogously to the “survival of the fittest” doctrine of natural selection. A detailed discussion of how this is achieved follows.

For each patient of the plurality of patients, the feature profile comprises a feature status for each of a plurality of features for that patient. The feature status may be represented in the form of a binary mask, in which a “1” indicates that a feature is present, and a “O” indicates that a feature is absent. The opposition configuration in which a “1” indicates that the feature is absent, and a “0” indicates that the feature is present is also covered by the present invention, albeit an unconventional arrangement. Similarly, for each individual generated using the genetic algorithm, the respective subset of features is represented in the form of a binary mask comprising all of the predetermined plurality of features, in which a “1” indicates that a feature is present and a “0” indicates that the feature is absent. Again, the inverse arrangement is also envisaged.

Herein, the feature may be genetic features. Specifically, the features may come any or all of three different forms:
The binary mask may comprise, for each of one or more genes, and indication whether there is a mutation at any point in that gene.
The binary mask may comprise, for at least one mutation, an indication of the type of mutation. Specifically, the binary mask may comprise an indication of whether the mutation is a gain-of-function mutation or a loss-of-function mutation. In this way, the genetic features may provide biological context for a mutation.
The binary mask may comprise, for each mutation, an indication of the position of that mutation within the gene in which it is located. Specifically, the indication of the position of that mutation comprises: for each of a plurality of hotspot locations within a given gene, an indication of whether a mutation is present at that hotspot location. In this way, the genetic features may provide biological context for a mutation.

Such binary masks may be used to predict the presence of a treatment response characteristic which indicates resistance to a treatment which has a defined molecular mechanism with a protein target e.g., certain cancer treatments such as those discussed above.

Herein, “hotspot” refers to a specific location within a gene in which mutations are common, or expected, and therefore which it is desirable to isolate and study using the genetic algorithm.

We now discuss how the fitness score may be calculated. For a given subset of features, represented by the feature profile, the fitness score may be calculated using an analytical model which evaluates the predictive power of a predictive model which uses only the features contained in the subset. As discussed, the purpose of the invention is to determine one or more set of features which may be used to predict the presence or absence of a particular phenotypic characteristic. This prediction may be effected by applying a predictive model to the set of features of a patient, an output of the predictive model indicative of whether the patient is likely to exhibit the phenotypic characteristic or not. This is the “predictive model” which we refer to above. The “analytical model” refers to a model which is used to determine the fitness score. The analytical model may be a machine-learning model, such as a binary classification model. In preferred implementations, the binary classification model is preferably a naïve Bayes model, which may have a Bernoulli prior. In those cases, the fitness score is preferably the cross-validation accuracy score of the naïve Bayes model on a training set which comprises a portion of the patient data (preferably the first subset of the patient data, as outlined earlier in this application). For improved results, the cross-validation accuracy is preferably class-balanced, and may be calculated using five folds.

We return to a detailed explanation of the steps which may be involved in the genetic algorithm.

In the first step of the algorithm, in step (b), it is preferred that the plurality of first generation G individuals are generated such that the subset of features of each respective individual comprises a predetermined proportion of the features of predetermined plurality of features. Alternatively, or additionally, the plurality of first generation Gᵢ individuals are generated in step (b) such that, across all of the first generation Gᵢ individuals, the subset of features of each respective individual comprises on average a predetermined proportion of the features of the predetermined plurality of features. Rather than an average, another statistical parameter may be used e.g. a median, mode, maximum, minimum, or a percentile. The predetermined proportion in this context is preferably tuneable. For example, the computer-implemented method may comprise receiving an input specifying the value of the predetermined proportion, and setting the predetermined proportion accordingly. The predetermined proportion may fall within a preferred range. The lower bound of the range may be 18, 2%, 38, 48, 58, 68, 78, 8%, or 98. The upper bound of the range may be 908, 808, 708, 60%, 50%, 40%, 30%, 208, 15%, 14%, 13%, 12% or 118. Preferably the predetermined proportion is about 10%. This may reflect the typical frequency of the occurrences of the features in real life patient data.

Genetic algorithms are typified by the use of techniques which mimic natural selection and evolution. Accordingly between one generation and the next, mutations may be applied to the individuals. In the context of the present invention, a mutation is a random (or pseudo-random) change in the feature status of one or more feature statuses within a feature profile. In order to implement this, generating the plurality of second-generation individuals may comprise, for each of one or more second generation individuals: sampling the plurality of first-generation individuals to select a candidate individuals, wherein the probability of a given first generation individual being sampled is based on the respective fitness score of that individual. Preferably, the probability is proportional to the fitness score for that individual. In this way, the individuals with the higher fitness score are more likely to be selected and “carried forward” to the next generation, mimicking the process of natural selection. The first parent individual should be different from the second parent individual. Then, generating the plurality of second-generation individuals may comprise mutating the subset of features of the candidate individual to generate a mutated subset of features, thereby generating a second-generation individual having as their subset of features the mutated subset of features. According to this method, a particular first-generation individual may form a starting point for more than one second generation individuals, again mirroring natural selection. Within the second generation of individuals, a first predetermined proportion of the total number of individuals may be generated by mutating the subset of features of a candidate individual. In other words, a fixed proportion of the individuals in the second generation are mutated versions of individuals in the first generation. The first predetermined proportion may be tuneable, and accordingly, the computer-implemented method may comprise receiving an input specifying the value of the first predetermined proportion, and setting the value of the first predetermined proportion accordingly. Preferred values of the first predetermined proportion will be set out later, after a second predetermined proportion has been introduced.

What is meant by mutation? In some cases, mutating the subset of features of the candidate individual may comprise randomly (or pseudo-randomly) adding or removing features from the subset of features. More specifically, where a feature is present in the subset of features, there is a first probability that it will be removed. Similarly, where a feature is absent from the subset of features, there is a second probability that it will be added. In preferred cases, the first probability is equal to the second probability. In other words, there is a fixed likelihood that the feature status of each feature will change. Preferably the first probability and/or the second probability is from 0.1% to 10%, and more preferably about 18. During the mutation step, features may be added and removed such that the total number of features in the mutated subset of features is the same as the number of features in the original subset of features.

As well as mutation, individuals in a subsequent generation may be generated by mating together individuals from the previous generation. Again, like biological natural selection, the individuals who have the highest fitness scores have a higher chance of “mating”. Accordingly, generating the plurality of second-generation individuals may comprise sampling the plurality of first-generation individuals to select a first parent individual and a second parent individual, wherein the generation of a given first generation individual being sampled is based on the respective fitness score of that individual. As before, the probability is preferably proportional to the fitness score. In this way, the individuals with the higher fitness score are more likely to be selected and “carried forward” to the next generation, mimicking the process of natural selection. After a first parent and a second parent have been selected from the first-generation individuals, generating the plurality of second-generation individuals may comprise mating the first parent individual and the second parent individual from the first generation, thereby generating a second-generation individual whose subset of feature is based on the respective subsets of features of the first parent individual and the second parent individual. As with mutation, within the second generation of individuals, a second predetermined proportion of the total number of individuals is generated by mating a first parent individual and a second parent individual. The second predetermined proportion may be tuneable, and accordingly, the computer-implemented method may comprise receiving an input specifying the value of the second predetermined proportion, and setting the value of the second predetermined proportion accordingly.

In some cases, all of the individuals in the second generation may have been generated either by mutation or mating of individuals in the first generation. In other words, the first predetermined proportion and the second predetermined proportion preferably sum to unity (i.e. to 100%). In preferred cases, the first predetermined proportion is greater than the second predetermined proportion. In implementation in which the first predetermined proportion and the second predetermined proportion do not add to 100%, the remaining proportion of the second generation may comprise randomly generated individuals (e.g. generated in the same manner as the first-generation individuals) and/or exact replicas of first-generation individuals. The first predetermined proportion may be 50% to 70%, or may be about 60%. The second predetermined proportion may be 30% to 50%, or may be about 40%.

What is meant by mating? Mating, in this context, refers to combining the subsets of features of the first parent individual and the second parent individual. More specifically, mating the first parent individual and the second parent individual comprises: for each of the predetermined plurality of features, selecting either the feature status of that feature from the first parent individual or the feature status of that feature from the second parent individual, as the feature status of that feature in the second-generation individual. It is preferable that the probability that the feature status will be selected from the first parent individual is equal to the probability that the feature status will be selected from the second parent individual. Alternatively, the probability that the feature will be selected from each parent individual maybe based (e.g. proportional to) the fitness score of that individual.

It should be noted that, in some implementations of the genetic algorithm, more than two first-generation individuals may be mated, in an analogous manner (i.e. by sampling a plurality of parent individuals, wherein in the probability of sampling each individual is based on the fitness score of that individual, and then selecting a feature from one of plurality of parent individuals).

The above disclosure explains the generation of a plurality of second-generation individuals from a plurality of first-generation individuals. It will be understood that processes for generating a plurality of i^{th}-generation individuals from a plurality of (i−1)^{th}-generation individuals may follow the same processes, where i≥2. However, in some cases, the process may be modified, since rather than taking account of the plurality of individuals in the immediately previous generation, the combined plurality of individuals in all previous generations may be considered.

We now set out some specific features in order to illustrate this.

Generating a plurality of i^{th}-generation individuals may comprise, for each of one or more of i^{th}-generation individuals: sampling the plurality of sampling the plurality of (i−1)^{th} generation individuals to select a candidate individual, wherein the probability of a given (i−1)^{th} generation individual being sampled is based on the respective fitness score for that individual. Then, the computer-implemented method may further comprise: mutating the subset of features of the candidate individual to generate a mutated subset of features, thereby generation an i^{th} generation individual having as their subset of features the mutated subset of features. The mutation process may take place in the same manner as outlined previously in this patent application. As outlined previously, within the i^{th} generation, a first predetermined proportion of the total number of individuals within the generation may be generated by mutating the subset of features of a candidate individual in the (i−1)^{th} generation.

In an alternative case, generating a plurality of i^{th}-generation may comprise, for each of one or more i^{th}-generation individuals, sampling a breeding pool of generated individuals to select a candidate individual, wherein the probability of an individual in the breeding pool being sampled is based on (e.g. proportional to) the respective fitness score for that individual. Accordingly, the computer-implemented method may comprise forming or otherwise generating the breeding pool. The breeding pool may contain one or more of the following: the plurality of individuals in the (i−1)^{th} generation; and a selected plurality of individuals from the (i−2) earlier generations Gⱼ, where j<i−1. Rather than a selection from the (i−2) generations, the breeding pool may contain a selected plurality of individuals from the K most recent generations, wherein K is a predetermined number of generations. The selected plurality of individuals preferably comprises a predetermined number of individuals from the set of all individuals from earlier generations whose fitness scores are the highest. Alternatively, or additionally, the selected plurality of individuals may contain a predetermined number of individuals from each generation, whose fitness scores are in a predetermined number of highest-ranking fitness scores in their respective generation. In this case, it is possible to maintain individuals from previous generations whose fitness scores are high. These individuals with high fitness scores may not be carried through to subsequent generations, as mutations/mating may result in feature profiles resulting in lower fitness scores than in previous generations. By selecting individuals from a breeding pool which contains individuals from all previous generations, this issue may be avoided. Within the i^{th} generation, a first determined number of individuals within the generation may be generated by mutation of a candidate individual from a previous generation.

A similar approach may be taken in respect of the mating process. Accordingly, generating a plurality of i^{th} generation individuals comprises, for each of one or more i^{th} generation individuals, selecting a first parent individual and a second parent individual from one or more previous generations of individuals. Then, the computer-implemented method may further comprise mating the first parent individual and the second parent individual from one or more previous generations, thereby generating an i^{th} generation individual whose subset of features is based on the respective subsets of features of the first parent individual and the second parent individual. As above, within the i^{th} generation, a second predetermined proportion of individuals within the generation may be generated by mating a first parent individual with a second parent individual. Selection of a first parent individual may comprise sampling the plurality of (i−1)th generation individuals to select the first parent individual, wherein the probability of a given (i−1)^{th} individual being selected is based on the respective fitness score of that individual. Selection of a second parent individual may comprise sampling the plurality of (i−1)^{th} generation individuals to select the second parent individual, wherein the probability of a given (i−1)^{th} individual being selected is based on the respective fitness score of that individual. In an alternative case, where the first and second parent individuals may be selected from any previous generation, selecting the first parent individual and the second parent individual may comprises: sampling a breeding pool of generated individuals to select the first parent individual and the second parent individual, wherein the probability of an individual in the breeding pool being sampled is based on the respective fitness score for that individual. The computer-implemented method may, accordingly, comprise forming or otherwise generating the breeding pool. The breeding pool may contain one or more of the following: the plurality of individuals in the (i−1)^{th} generation; and a selected plurality of individuals from the (i−2) earlier generations Gⱼ, where j<i−1. Rather than a selection from the (i−2) generations, the breeding pool may contain a selected plurality of individuals from the K most recent generations, wherein K is a predetermined number of generations. The selected plurality of individuals preferably comprises a predetermined number of individuals from the set of all individuals from earlier generations whose fitness scores are the highest. Alternatively, or additionally, the selected plurality of individuals may contain a predetermined number of individuals from each generation, whose fitness scores are in a predetermined number of highest-ranking fitness scores in their respective generation. In this case, it is possible to maintain individuals from previous generations whose fitness scores are high.

It has been observed by the inventors that the use of three distinct types of features, more specifically genetic features, gives rise to advantageous results in terms of e.g. granularity. Accordingly, a second aspect of the present invention provides a computer-implemented method of determining one or more sets of genetic features to predict the presence of a particular phenotypic characteristic, the computer-implemented method comprising: (a) receiving patient data comprising, for each of a plurality of patients: for each of a plurality of genetic features, binary mask indicating whether that genetic feature is present or absent in the genome of the patient, the binary mask comprising: for each or one or more genes, an indication whether there is a mutation at any point in that gene; for each mutation, an indication whether the mutation is a gain-of-function or loss-of-function mutation; and for each of a plurality of hotspot locations within a gene, an indication whether a mutation is present at that location; and an indication of whether that patient expresses the particular phenotypic characteristic; (b) using a genetic algorithm to generate a plurality of generations of individuals, wherein each individual comprises a subset of the predetermined plurality of features, each generation of individuals generated based, at least in part, on a plurality of fitness scores, each fitness score corresponding to a respective individual in the previous generation, and indicative of how well the set of features of that individual are able to predict the presence or absence of the phenotypic characteristic, each fitness score being calculated based at least in part on the patient data; (c) repeating step (b) until it has been performed N times; (d) from the plurality of individuals generated in steps (b) and (c), identifying, based at least in part on the respective fitness scores of the individuals, one or more sets of genetic features to predict the presence of a particular phenotypic characteristic. All features which have been set out above (either those features of the first aspect of the invention, or the optional features), particularly those features which relate to the clustering process used to identify the sets of features, may also be combined with the second aspect of the invention.

Up to this point, the disclosure focuses on the identification of a set of features which may be used as predictors of a particular phenotypic condition. We now discuss how these predictors may be used once they have been determined. It should be noted that the sets of features (i.e. the predictors) may have been obtained using either the computer-implemented method of the first aspect of the invention, or the computer-implemented method of the second aspect of the invention; both approaches are equally valid, and neither is preferable.

A third aspect of the invention provides a computer-implemented method of generating an analytical model for predicting the presence or absence of a particular phenotypic characteristic, the computer-implemented invention comprising: determining one or more sets of features using the computer-implemented method of the first aspect of the invention or the second aspect of the invention; and training an analytical model using training data relating to the one or more sets of features to generate a trained analytical model. The analytical model is preferably a machine-learning model, such as a binary classification model. The binary classification model may be a naïve Bayes model, which may in turn comprise a Bernoulli prior. The training data may comprise a feature profile which is a genetic feature profile having similar characteristics to a genetic feature profile which may be used for identifying the feature sets, i.e. the received genetic feature profile comprises a binary mask, the binary mask comprising: for each of one or more genes, an indication of whether there is a mutation at any point in that gene; and for each mutation, at least one of: (1) an indication of whether the mutation is a gain-of-function mutation or a loss-of-function mutation; (2) an indication of the position of that mutation within the gene in which it is located, the indication comprising, for each of a plurality of hotspot locations within a given gene, an indication of whether the mutation is present at that hotspot.

A fourth aspect of the invention provides a computer-implemented method of predicting whether a patient is likely to display a particular phenotypic condition, the computer-implemented method comprising: receiving a feature profile containing a feature status of each of an identified set of features; applying the analytical model generated according to the computer-implemented method of the third aspect of the invention to the received feature profile; and outputting a result indicative of whether the patient is likely to display the particular phenotypic condition. The feature profile may be a genetic feature profile having similar characteristics to a genetic feature profile which may be used for identifying the feature sets, i.e. the received genetic feature profile comprises a binary mask, the binary mask comprising: for each of one or more genes, an indication of whether there is a mutation at any point in that gene; and for each mutation, at least one of: (1) an indication of whether the mutation is a gain-of-function mutation or a loss-of-function mutation; (2) an indication of the position of that mutation within the gene in which it is located, the indication comprising, for each of a plurality of hotspot locations within a given gene, an indication of whether the mutation is present at that hotspot.

Additional aspects of the invention provide:
A system comprising a processor configured to execute the computer-implemented method of the first aspect of the invention.
A system comprising a processor configured to execute the computer-implemented method of the second aspect of the invention.
A system comprising a processor configured to execute the computer-implemented method of the third aspect of the invention.
A system comprising a processor configured to execute the computer-implemented method of the fourth aspect of the invention.
A computer program comprising instructions, which when the program is executed by a computer, or a processor thereof, causes the computer to carry out the computer-implemented of the first aspect of the invention.
A computer program comprising instructions, which when the program is executed by a computer, or a processor thereof, causes the computer to carry out the computer-implemented of the second aspect of the invention.
A computer program comprising instructions, which when the program is executed by a computer, or a processor thereof, causes the computer to carry out the computer-implemented of the third aspect of the invention.
A computer program comprising instructions, which when the program is executed by a computer, or a processor thereof, causes the computer to carry out the computer-implemented of the fourth aspect of the invention.
A computer-readable storage medium comprising instructions which, when executed by a computer, cause the computer to execute the computer-implemented method of the first aspect of the invention.
A computer-readable storage medium comprising instructions which, when executed by a computer, cause the computer to execute the computer-implemented method of the second aspect of the invention.
A computer-readable storage medium comprising instructions which, when executed by a computer, cause the computer to execute the computer-implemented method of the third aspect of the invention.
A computer-readable storage medium comprising instructions which, when executed by a computer, cause the computer to execute the computer-implemented method of the fourth aspect of the invention.

The invention includes the combination of the aspects and preferred features described except where such a combination is clearly impermissible or expressly avoided.

In addition to the above, the following disclosure provides clarifications of some terms which may be used throughout this patent application.

A “sample” as used herein may be a cell or tissue sample, a biological fluid, an extract (e.g. a DNA extract obtained from the subject), from which genomic material can be obtained for genomic analysis, such as genomic sequencing (e.g. whole genome sequencing, whole exome sequencing). The sample may be a cell, tissue or biological fluid sample obtained from a subject (e.g. a biopsy). Such samples may be referred to as “subject samples”. In particular, the sample may be a blood sample, or a tumour sample, or a sample derived therefrom. The sample may be one which has been freshly obtained from a subject or may be one which has been processed and/or stored prior to genomic analysis (e.g. frozen, fixed or subjected to one or more purification, enrichment or extraction steps). The sample may be a cell or tissue culture sample. As such, a sample as described herein may refer to any type of sample comprising cells or genomic material derived therefrom, whether from a biological sample obtained from a subject, or from a sample obtained from e.g. a cell line. In embodiments, the sample is a sample obtained from a subject, such as a human subject. The sample is preferably from a mammalian (such as e.g. a mammalian cell sample or a sample from a mammalian subject, such as a cat, dog, horse, donkey, sheep, pig, goat, cow, mouse, rat, rabbit or guinea pig), preferably from a human (such as e.g. a human cell sample or a sample from a human subject). Further, the sample may be transported and/or stored, and collection may take place at a location remote from the genomic sequence data acquisition (e.g. sequencing) location, and/or any computer-implemented method steps described herein may take place at a location remote from the sample collection location and/or remote from the genomic data acquisition (e.g. sequencing) location (e.g. the computer-implemented method steps may be performed by means of a networked computer, such as by means of a “cloud” provider).

The subject may have a cancer which comprises a solid tumour (primary and/or metastatic In some cases, the cancer may be a cancer for which CPI therapy has been approved as a treatment option. In particular, the cancer may comprise Advanced Urothelial Carcinoma, Breast Cancer, Colorectal Cancer, Advanced Endometrial Cancer, Gastric Cancer, Hepatocellular Carcinoma, Head and Neck Cancer, Melanoma, Malignant Pleural Mesothelioma, Non-Small Cell Lung Cancer (NSCLC), Renal Cell Carcinoma or Small-Cell Lung Cancer. In some cases, the cancer may be a cancer for which CPI therapy has not (yet) been approved as a treatment option. In particular, the cancer may be selected from Acute Myeloid Leukemia, Chronic Lymphocytic Leukemia, Diffuse Large B-Cell Lymphoma, Follicular Lymphoma, Mantle Cell Lymphoma, Multiple Myeloma, Ovarian Cancer, Metastatic Pancreatic Cancer, and Metastatic Prostate Cancer.

A “mixed sample” refers to a sample that is assumed to comprise multiple cell types or genetic material derived from multiple cell types. Within the context of the present disclosure, a mixed sample is typically one that comprises tumour cells, or is assumed (expected) to comprise tumour cells, or genetic material derived from tumour cells. Samples obtained from subjects, such as e.g. tumour samples, are typically mixed samples (unless they are subject to one or more purification and/or separation steps). Typically, the sample comprises tumour cells and at least one other cell type (and/or genetic material derived therefrom). For example, the mixed sample may be a tumour sample. A “tumour sample” refers to a sample derived from or obtained from a tumour. Such samples may comprise tumour cells and normal (non-tumour) cells. The normal cells may comprise immune cells (such as e.g. lymphocytes), and/or other normal (non-tumour) cells. The lymphocytes in such mixed samples may be referred to as “tumour-infiltrating lymphocytes” (TIL). A tumour may be a solid tumour or a non-solid or haematological tumour. A tumour sample may be a primary tumour sample, tumour-associated lymph node sample, or a sample from a metastatic site from the subject. A sample comprising tumour cells or genetic material derived from tumour cells may be a bodily fluid sample. Thus, the genetic material derived from tumour cells may be circulating tumour DNA or tumour DNA in exosomes. Instead or in addition to this, the sample may comprise circulating tumour cells. A mixed sample may be a sample of cells, tissue or bodily fluid that has been processed to extract genetic material. Methods for extracting genetic material from biological samples are known in the art. A mixed sample may have been subject to one or more processing steps that may modify the proportion of the multiple cell types or genetic material derived from the multiple cell types in the sample. For example, a mixed sample comprising tumour cells may have been processed to enrich the sample in tumour cells. Thus, a sample of purified tumour cells may be referred to as a “mixed sample” on the basis that small amounts of other types of cells may be present, even if the sample may be assumed, for a particular purpose, to be pure (i.e. to have a tumour fraction of 1 or 100%).

A “normal sample” or “germline sample” refers to a sample that is assumed not to comprise tumour cells or genetic material derived from tumour cells. A germline sample may be a blood sample, a tissue sample, or a purified sample such as a sample of peripheral blood mononuclear cells from a subject. Similarly, the terms “normal”, “germline” or “wild type” when referring to sequences or genotypes refer to the sequence/genotype of cells other than tumour cells. A germline sample may comprise a small proportion of tumour cells or genetic material derived therefrom, and may nevertheless be assumed, for practical purposes, not to comprise said cells or genetic material. In other words, all cells or genetic material may be assumed to be normal and/or sequence data that is not compatible with the assumption may be ignored.

The term “sequence data” refers to information that is indicative of the presence and preferably also the amount of genomic material in a sample that has a particular sequence. Such information may be obtained using sequencing technologies, such as e.g. next generation sequencing (NGS), for example whole exome sequencing (WES), whole genome sequencing (WGS), or sequencing of captured genomic loci (targeted or panel sequencing), or using array technologies, such as e.g. copy number variation arrays, or other molecular counting assays. When NGS technologies are used, the sequence data may comprise a count of the number of sequencing reads that have a particular sequence. When non-digital technologies are used such as array technology, the sequence data may comprise a signal (e.g. an intensity value) that is indicative of the number of sequences in the sample that have a particular sequence, for example by comparison to an appropriate control. Sequence data may be mapped to a reference sequence, for example a reference genome, using methods known in the art (such as e.g. Bowtie (Langmead et al., 2009)). Thus, counts of sequencing reads or equivalent non-digital signals may be associated with a particular genomic location (where the “genomic location” refers to a location in the reference genome to which the sequence data was mapped). Further, a genomic location may contain a mutation, in which case counts of sequencing reads or equivalent non-digital signals may be associated with each of the possible variants (also referred to as “alleles”) at the particular genomic location. The process of identifying the presence of a mutation at a particular location in a sample is referred to as “variant calling” and can be performed using methods known in the art (such as e.g. the GATK HaplotypeCaller, https://gatk.broadinstitute.org/hc/en-us/articles/360037225632-HaplotypeCaller). For example, sequence data may comprise a count of the number of reads (or an equivalent non-digital signal) which match a germline (also sometimes referred to as “reference”) allele at a particular genomic location, and a count of the number of reads (or an equivalent non-digital signal) which match a mutated (also sometimes referred to as “alternate”) allele at the genomic location.

Further, sequence data may be used to infer copy number profiles along a genome, using methods known in the art. Copy number profiles may be allele specific. In the context of the present invention, copy number profiles are preferably allele specific and tumour/normal sample specific. In other words, the copy number profiles used in the present invention are preferably obtained using methods designed to analyse samples comprising a mixture of tumour and normal cells, and to produce allele-specific copy number profiles for the tumour cells and the normal cells in a sample. Allele specific copy number profiles for mixed samples may be obtained from sequence data (e.g. using read counts as described above), using e.g. ASCAT (Van Loo et al., 2010). Other methods are known and equally suitable. Preferably, within the context of the present invention, the method used to obtain allele-specific copy number profiles is one that reports a plurality of possible copy number solutions and an associated quality/confidence metric. For example, ASCAT outputs a goodness-of-fit metric for each combination of values of ploidy (ploidy for a whole tumour sample, not segment-specific) and purity for which a corresponding allele-specific copy number profile was evaluated. Note that the tumour-specific copy number profiles generated by such methods represent an average or summary of the entire tumour cell population (i.e. it does not account for heterogeneity within the tumour population).

The term “total copy number” refers to the total number of copies of a genomic region in a sample. The term “major copy number” refers to the number of copies of the most prevalent allele in a sample. Conversely, the term “minor copy number” refers to the number of copies of the allele other than the most prevalent allele in a sample. Unless indicated otherwise, these terms refer to the inferred major and major copy numbers (and total copy numbers) for an inferred tumour copy number profile. The term “normal copy number” or “normal total copy number” refers to the number of copies of a genomic region in the normal cells in a sample. Normal cells typically have two copies of each chromosome (unless the cell is genetically male and the chromosome is a sex chromosome), and hence the normal copy number may in embodiments be assumed to be equal to 2 (unless the genomic region is on the X or Y chromosome and the sample under analysis is from a male subject, in which case the normal copy number may be assumed to be equal to 1). Alternatively, the normal copy number for a particular genomic region may be determined using a normal sample.

### Methods for Classification Based on Gene Mutations

In some embodiments, the present invention provides methods for classifying, prognosticating, predicting treatment response (e.g. to CPI therapy) or monitoring cancer in subjects. In particular, data obtained from analysis DNA sequencing may be evaluated using one or more pattern recognition algorithms. Such analysis methods may be used to form a predictive model, which can be used to classify test data. For example, one convenient and particularly effective method of classification employs multivariate statistical analysis modelling, first to form a model (a “predictive mathematical model”) using data (“modelling data”) from samples of known subgroup (e.g., from subjects known to have a particular CPI response), and second to classify an unknown sample (e.g., “test sample”) to the appropriate response group.

Pattern recognition methods have been used widely to characterize many different types of problems ranging, for example, over linguistics, fingerprinting, chemistry and psychology. In the context of the methods described herein, pattern recognition is the use of multivariate statistics, both parametric and non-parametric, to analyse data, and hence to classify samples and to predict the value of some dependent variable based on a range of observed measurements. There are two main approaches. One set of methods is termed “unsupervised” and these simply reduce data complexity in a rational way and also produce display plots which can be interpreted by the human eye. However, this type of approach may not be suitable for developing a clinical assay that can be used to classify samples derived from subjects independent of the initial sample population used to train the prediction algorithm.

The other approach is termed “supervised” whereby a training set of samples with known class or outcome is used to produce a mathematical model which is then evaluated with independent validation data sets. Here, a “training set” of mutation data is used to construct a statistical model that predicts correctly the “subgroup” of each sample. This training set is then tested with independent data (referred to as a test or validation set) to determine the robustness of the computer-based model. These models are sometimes termed “expert systems,” but may be based on a range of different mathematical procedures such as support vector machine, decision trees, k-nearest neighbour and naïve Bayes. Supervised methods can use a data set with reduced dimensionality (for example, the first few principal components), but typically use unreduced data, with all dimensionality. In all cases the methods allow the quantitative description of the multivariate boundaries that characterize and separate each subtype in terms of its intrinsic mutation profile. It is also possible to obtain confidence limits on any predictions, for example, a level of probability to be placed on the goodness of fit. The robustness of the predictive models can also be checked using cross-validation, by leaving out selected samples from the analysis.

The terms “tumour-specific mutation”, “somatic mutation” or simply “mutation” are used interchangeably and refer to a difference in a nucleotide sequence (e.g. DNA or RNA) in a tumour cell compared to a healthy cell from the same subject. A germline mutation, by contrast, occurs in germ cells and is passed on to offspring, such that the mutation is present in essentially all cells of the individual. A germline mutation may be a mutation that predisposes the individual carrying the mutation to developing a cancer (e.g. a mutation in the gene TP53, or the BRCA1 gene or BRCA2 gene).

As a result of a somatic mutation, the difference in the nucleotide sequence can result in the expression of a protein which is not expressed by a healthy cell from the same subject. For example, a mutation may be a single nucleotide variant (SNV), multiple nucleotide variant (MNV), a deletion mutation, an insertion mutation, a translocation, a missense mutation, a translocation, a fusion, a splice site mutation, or any other change in the genetic material of a tumour cell. A mutation may result in the expression of a protein or peptide that is not present in a healthy cell from the same subject. Mutations may be identified by exome sequencing, RNA-sequencing, whole genome sequencing and/or targeted gene panel sequencing and or routine Sanger sequencing of single genes, followed by sequence alignment and comparing the DNA and/or RNA sequence from a tumour sample to DNA and/or RNA from a reference sample or reference sequence (e.g. the germline DNA and/or RNA sequence, or a reference sequence from a database). Suitable methods are known in the art.

As used herein a “gain of function” or “GOF” mutation may be a high frequency mutation (HFM) as defined herein. Therefore, GOF and HFM may be used interchangeably. A “loss of function” or “LOF” mutation may be a low frequency mutation (LFM) as defined herein. Therefore, LOF and LFM may be used interchangeably. In particular, HFM and LFM (and GOF/LOF, accordingly) may be defined according to the following classification scheme: 1. the total number of amino acids mutated per gene was calculated; 2. the frequency of mutations in each gene was calculated (i.e., how many patients had any mutation in that gene). 3. From #1 and #2 the average amino acid mutation rate was calculated:

Average amino acid mutation=(gene level mutation frequency (#2))/(Total amino acids mutated in the gene (#1))

4. The HFM label was assigned to any mutation that had 2× the average mutations per that specific amino acid and had more than/equal to 9 mutations in that gene. The LFM label was assigned to any mutation that had lower than 2× the average mutations per that specific amino acid and/or had less than 9 mutations. Any mutation in the TERT promoter was classified as HFM. Amplifications were considered as HFM and deletions as LFM. The rationale behind this was that LFM tend to be loss of function (LOF) and HFM tend to be gain of function (GOF).

The Hotspot granular classification as used herein employs the same definition as described above for HFM/LFM, but adds the amino acid mutation location to any HFM. For example, TP53 178, refers to a HFM in TP53 located at amino acid 178, wherein the amino acid position number refers to the encoded protein sequence. Any HFM that lacks the information about amino acid location is defined as an amplification mutation. The patient population in which the determinations of high frequency or low frequency, as set out above, may be a population such as the approximately 10,000 non-small cell lung cancer patients from the Flatiron Health-Foundation Medicine NSCLC de-identified clinico-genomics database (JAMA 2019; 321 (14): 1391-1399. doi: 10.1001/jama.2019.3241), TCGA datasets (https://www.cancer.gov/about-nci/organization/ccg/research/structural-genomics/tcga) and/or from an internal clinic-genomics database. In particular, the patient population may be that described in Singal G, Miller PG, Agarwala V, et al. Association of Patient Characteristics and Tumor Genomics With Clinical Outcomes Among Patients With Non-Small Cell Lung Cancer Using a Clinicogenomic Database (CGDB). JAMA. 2019; 321 (14): 1391-1399.

doi: 10.1001/jama.2019.3241 (the entire contents of which is expressly incorporated herein by reference, including the de-identified CGDB).

An “indel mutation” refers to an insertion and/or deletion of bases in a nucleotide sequence (e.g. DNA or RNA) of an organism. Typically, the indel mutation occurs in the DNA, preferably the genomic DNA, of an organism. An indel mutation may be a frameshift indel mutation. A frameshift indel mutation is a change in the reading frame of the nucleotide sequence caused by an insertion or deletion of one or more nucleotides. Such frameshift indel mutations may generate a novel open-reading frame which is typically highly distinct from the polypeptide encoded by the non-mutated DNA/RNA in a corresponding healthy cell in the subject.

A “neoantigen” (or “neo-antigen”) is an antigen that arises as a consequence of a mutation within a cancer cell. Thus, a neoantigen is not expressed (or expressed at a significantly lower level) by normal (i.e. non-tumour) cells. A neoantigen may be processed to generate distinct peptides which can be recognised by T cells when presented in the context of MHC molecules. Neoantigens may be used as the basis for cancer immunotherapies. References herein to “neoantigens” are intended to include also peptides derived from neoantigens. The term “neoantigen” as used herein is intended to encompass any part of a neoantigen that is immunogenic. An “antigenic” molecule as referred to herein is a molecule which itself, or a part thereof, is capable of stimulating an immune response, when presented to the immune system or immune cells in an appropriate manner. The binding of a neoantigen to a particular MHC molecule (encoded by a particular HLA allele) may be predicted using methods which are known in the art. Examples of methods for predicting MHC binding include those described by Lundegaard et al., O'Donnel et al., and Bullik-Sullivan et al. For example, MHC binding of neoantigens may be predicted using the netMHC-3 (Lundegaard et al.) and netMHCpan4 (Jurtz et al.) algorithms. A neoantigen that has been predicted to bind to a particular MHC molecule is thereby predicted to be presented by said MHC molecule on the cell surface.

A cancer immunotherapy (or simply “immunotherapy”) refers to a therapeutic approach comprising administration of an immunogenic composition (e.g. a vaccine), a composition comprising immune cells, or an immunoactive drug, such as e.g. a therapeutic antibody, to a subject. The term “immunotherapy” may also refer to the therapeutic compositions themselves. In the context of the present invention, the immunotherapy typically targets a neoantigen. For example, an immunogenic composition or vaccine may comprise a neoantigen, neoantigen presenting cell or material necessary for the expression of the neoantigen. As another example, a composition comprising immune cells may comprise T and/or B cells that recognise a neoantigen. The immune cells may be isolated from tumours or other tissues (including but not limited to lymph node, blood or ascites), expanded ex vivo or in vitro and re-administered to a subject (a process referred to as “adoptive cell therapy”). Instead or in addition to this, T cells can be isolated from a subject and engineered to target a neoantigen (e.g. by insertion of a chimeric antigen receptor that binds to the neoantigen) and re-administered to the subject. As another example, a therapeutic antibody may be an antibody which recognises a neoantigen.

A composition as described herein may be a pharmaceutical composition which additionally comprises a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

References to “an immune cell” are intended to encompass cells of the immune system, for example T cells, NK cells, NKT cells, B cells and dendritic cells. In a preferred embodiment, the immune cell is a T cell. An immune cell that recognises a neoantigen may be an engineered T cell. A neoantigen specific T cell may express a chimeric antigen receptor (CAR) or a T cell receptor (TCR) which specifically binds a neoantigen or a neoantigen peptide, or an affinity-enhanced T cell receptor (TCR) which specifically binds a neoantigen or a neoantigen peptide (as discussed further hereinbelow). For example, the T cell may express a chimeric antigen receptor (CAR) or a T cell receptor (TCR) which specifically binds to a neo-antigen or a neo-antigen peptide (for example an affinity enhanced T cell receptor (TCR) which specifically binds to a neo-antigen or a neo-antigen peptide). Alternatively, a population of immune cells that recognise a neoantigen may be a population of T cell isolated from a subject with a tumour. For example, the T cell population may be generated from T cells in a sample isolated from the subject, such as e.g. a tumour sample, a peripheral blood sample or a sample from other tissues of the subject. The T cell population may be generated from a sample from the tumour in which the neoantigen is identified. In other words, the T cell population may be isolated from a sample derived from the tumour of a patient to be treated, where the neoantigen was also identified from a sample from said tumour. The T cell population may comprise tumour infiltrating lymphocytes (TIL).

The term “Antibody” (Ab) includes monoclonal antibodies, polyclonal antibodies, multispecific antibodies (e.g., bispecific antibodies), and antibody fragments that exhibit the desired biological activity. The term “immunoglobulin” (Ig) may be used interchangeably with “antibody”. Once a suitable neoantigen has been identified, for example by a method according to the invention, methods known in the art can be used to generate an antibody.

An “immunogenic composition” is a composition that is capable of inducing an immune response in a subject. The term is used interchangeably with the term “vaccine”. The immunogenic composition or vaccine described herein may lead to generation of an immune response in the subject. An “immune response” which may be generated may be humoral and/or cell-mediated immunity, for example the stimulation of antibody production, or the stimulation of cytotoxic or killer cells, which may recognise and destroy (or otherwise eliminate) cells expressing antigens corresponding to the antigens in the vaccine on their surface.

As used herein “treatment” refers to reducing, alleviating or eliminating one or more symptoms of the disease which is being treated, relative to the symptoms prior to treatment. “Prevention” (or prophylaxis) refers to delaying or preventing the onset of the symptoms of the disease. Prevention may be absolute (such that no disease occurs) or may be effective only in some individuals or for a limited amount of time.

As used herein, the terms “computer system” includes the hardware, software and data storage devices for embodying a system or carrying out a method according to the above described embodiments. For example, a computer system may comprise a central processing unit (CPU), input means, output means and data storage, which may be embodied as one or more connected computing devices. Preferably the computer system has a display or comprises a computing device that has a display to provide a visual output display (for example in the design of the business process). The data storage may comprise RAM, disk drives or other computer readable media. The computer system may include a plurality of computing devices connected by a network and able to communicate with each other over that network. It is explicitly envisaged that computer system may consist of or comprise a cloud computer.

As used herein, the term “computer readable media” includes, without limitation, any non-transitory medium or media which can be read and accessed directly by a computer or computer system. The media can include, but are not limited to, magnetic storage media such as floppy discs, hard disc storage media and magnetic tape; optical storage media such as optical discs or CD-ROMs; electrical storage media such as memory, including RAM, ROM and flash memory; and hybrids and combinations of the above such as magnetic/optical storage media.

### BRIEF DESCRIPTION OF THE DRAWINGS

Embodiments of the present invention will now be described with reference to the accompanying drawings, in which:

FIG. 1: Patient's treatment outcome group definition and cohort selection from CGDB
A. Schematic representation of response definition for durable-response and innate-resistance. The long blue arrows represent patient journey over time. The study period (270 days) in which treatment outcomes were investigated is marked by green dotted lines. Green vertical arrows represent the first time a patient was treated with a CPI and the grayed out area represents buffer time (14 days) during which treatment outcomes are ignored because they were likely resulting from the previous treatment. Green circle represents clinical benefit from CPI therapy (CR, PR, SD), while red X represents disease progression. B. Schematic representing the number of patients selected based on the criteria depicted in the scheme (more details in methods) C. Clinical characteristics of the cohort

FIG. 2: Mutational landscape of the selected cohort
A. Oncoplot including all patients in the CPI cohort (n=799), depicting the top 12 altered genes and some clinical characteristics. Each column represents a single patient and each row represents a gene. The bar plot on the top of the figure represents the number of mutations in each patient with each color representing the type of mutations (Missense, splice site, frame shift, etc). Middle of the figure represents a heatmap with clinical characteristics that include response, histology, smoking status, gender and ancestry call, with color scheme depicted at the bottom of the figure. The bottom part represents the oncoplot with different colors in each row representing a specific mutation in the depicted patient. The different colors represent different types of mutations (color scheme as in A) with the color scheme depicted at the bottom of the figure. The right side of the oncoplot shows a bar graph summarizing the total count of the indicated mutation types and the stacking represents the proportion of each type of mutation. B. Bar graph showing the top short variants found in our cohort (n=799) with color stacking representing the different types of mutation within each gene (same color scheme as in A) C. The left panel shows prevalence (in percent) of the top 10 deleted genes (green bar graph) and lower left panel is showing prevalence (in percent) of top 10 amplified genes (orange bar graph). D. Bar graph representing prevalence (in percent) of top 10 rearrangements, with color scheme (bottom figure legend) representing different types of rearrangements.

FIG. 3: Mutation association with response or resistance
Tables representing mutations that were found to be significantly or marginally-significantly enriched with treatment outcome (durable-response or innate-resistance). The first section (depicted as single gene) represents statistical test results on single gene levels analysing the three different mutation classifications (binary, HFM/LFM, Hotspot). The middle section (depicted as pair co-occurrence) represents statistical test results on a pair of co-occurring mutations showing results from binary mutation classification. Bottom section (represented as triplet co-occurrence) represents statistical test results on triplet co-occurring mutations showing results from binary mutation classification. For each section genes associated with durable-response are green, and genes associated with innate-resistance are red. Columns show: Mutation, showing the mutated gene name. P-value, showing p-value derived by Fisher Exact Test. Corrected P-value, showing false discovery rate (FDR) corrected p-value. DR with mutations, shows the number of durable-response (DR) patients with mutation in the gene of interest (among 799 patients), and in brackets the percent of patients having the mutation with DR (#DR/#IR+ #DR). IR with mutation, shows the number of innate-resistant (IR) patients with the specific mutation in the gene of interest, in brackets same as in DR with mutation column. Freq %, represents the percent of patients having any mutation in the specific gene, calculated across 8768 patients. Any gene/row with FDR value below 0.05, was filled in green.

FIG. 4: OS analysis of patients with mutations found to be significantly associated with CPI response
A. Kaplan-Meier survival curves of overall survival (OS) in patients treated with CPI or Chemo with or without mutations in genes found to be significant/marginally-significant in FIG. 3. Each Kaplan-Meier plot also shows the number of patients (in the Number at risk table) at each time point (Time in month), and includes a significance table with p-values when comparing each patient group (depicted as Group1 and Group2) at the bottom of each plot. B. Same as in A, with the exception of using the extended CGDB database of 3362 patients treated with CPI and Chemo.

FIG. 5: ML pipeline identifies 36 predictive mutation signatures with a core of shared genes
A. Schematic depicting the machine learning pipeline. B. Plot showing the Area under the ROC Curve (AUC) from held out (not used in training) test set (n=121) for each of the input types (Binary, HFM/LFM and Hotspot granular) for 36 different mutation signatures with blue and orange dots representing CPI and Chemo treated patients respectively. Each blue dot represents AUC derived from held out test set for an individual mutation signature in patients treated with CPI therapy, while orange dots represent Area under ROC Curve score derived from same mutation signature but in patients treated with chemotherapy. Error bars in blue and orange were derived from cross validation scores and are standard error of the mean (SEM). For each graph the black dotted horizontal line represents the scores for only TMB model and the orange dotted line represents the 50 percent accuracy score (depicted as random chance). Lower right panel shows average AUC scores of the 12 mutation signatures for each of the three inputs, with error bars as SEM. C. Left Venn diagram representing the gene overlap between the 36 mutation signatures across binary, HFM/LFM and Hotspot granular inputs, with 8 genes representing genes that are included in every single mutation signature (36). The right Venn diagram represents overlap between the unique genes within the 12 mutation signatures across the three inputs (58 binary genes, 149 HFM/LFM genes, and 165 hotspot genes).

FIG. 6: Relative contribution of mutations to CPI response in 36 mutation signatures
A. Waterfall plot of the top 10 linear coefficients (represent feature importance) derived from linear conversion of the 36 ML derived mutation signatures sorted by feature importance with positive values indicate association with durable-response (green bars) and negative values with innate-resistance (red bars). Left panel represents all the unique genes in binary input. Middle panel represents HFM/LFM feature importance and right most panel represents feature importance from Hotspot granular input. B. Plot representing linear coefficients in genes in which HFM and LFM mutations have divergent effects on CPI response, with red associated with innate-resistance and blue with durable-response.

FIG. 7: Pathway analysis of predictive mutation signatures reveals immune response and other biological pathways associated with CPI response.
Top 10 pathways derived from CBDD pathway analysis utilizing 8 different network-based algorithms. The results represent the pathways that have lowest P-value (derived from hypergeometric test) across algorithms. The Binary and HFM/LFM mutations are the two top panels. Lower panel (depicted as Overlap of Binary, HFM/LFM and Hotspot) represents the topology assisted pathway analysis of the 39 gene overlap between binary, HFM/LFM and Hotspot granular mutation signatures.

FIG. 8: Validating the role of IL6 identified from the pathway analysis at the protein level in atezolizumab clinical study. High serum levels of IL-6 is associated with progressive disease in patients treated with Atezolizumab
A. Boxplot depicting IL6 levels at baseline in patients who atezolizumab response was assessed by the RECIST criteria (CR, PR, SD or PD). B. Kaplan-Meier curves of OS in patients treated with Atezolizumab (trial PCD4989G) comparing patients with high (red) and low (blue) IL-6 serum levels, with the number of patient's in each month shown in the Number in risk table below the OS plot.

FIG. 9: Mutational landscape of the selected cohort grouped by response, and OS analysis of PDGFRB
A. Oncoplot of the CPI cohort (n=799) depicting the top 12 altered genes and selected clinical characteristics, segregated by response (depicted as response in lower part of oncoplot). Each column represents a single patient and each row represents a specific gene (name of gene listed on the left side). The top of the figure the histogram represents the number of mutations in each patient with each color representing the type of mutations (as indicated in figure). The middle part represents the oncoplot with different colors in each row represent a specific mutation in a specific patient, and different colors represent different types of mutations (as indicated in the figure), with the right side of the oncoplot showing a bar graph summarizing the number of mutations and the proportion of each type of mutation. Bottom of the figure represents selected clinical characteristics that include response group, histology, smoking status, gender and ancestry call, with color scheme depicted in figure legend. B. Stacked bar plot comparing prevalence of top 12 mutations between durable response (left) and innate-resistance (right side). Each color in the stacked bar plot represents a different type of mutations with same color scheme depicted in A. C. Kaplan-Meier survival overall survival (OS) curve in patients treated with CPI or Chemo for PDGFRB, details same as in FIG. 4.

FIG. 10: OS analysis of patients with mutations found to be significantly associated with CPI response
A. Kaplan-Meier survival curves of overall survival (OS) in patients treated with CPI or Chemo with or without mutations in genes found to be significant/marginally-significant in FIG. 3. Each Kaplan-Meier plot also shows the number of patients (in the Number at risk table) at each time point (Time in month), and includes a significance table with p-values when comparing each patient group (depicted as Group1 and Group2) at the bottom of each plot. B. Same as in A, with the exception of using the extended CGDB database of 3362 patients treated with CPI and Chemo.

FIG. 11: Accuracy and diversity of the 36 predictive mutation signatures
Heatmap representing the Pearson Correlation between the 12 mutation signatures within each of the three input classifications (binary, HFM/LFM and Hotspot granular). To the right of the heatmaps (the “Overlap” middle column) are genes depicted as overlap, between the 12 mutation signatures within each input category. The last column represents the overlap between the 36 mutation signatures across the three input categories.

FIG. 12: Linear conversion of 36 predictive models reveals feature importance (full list)
Waterfall plot of all the linear coefficients (depicted as feature importance) derived from linear conversion of the 36 ML derived mutation signatures sorted by feature importance with positive values indicate association with durable-response (green bars) and negative values indicate association with innate-resistance (red bars). Top panel represents all the unique genes in binary input separated by features contributing to durable response (green) and features contributing to resistance (red). Middle panel represents HFM/LFM feature importance and bottom panel represents feature importance from Hotspot granular input. Black vertical line separates between the responses.

FIG. 13: Mean accuracy across five training/validation splits.
The plot shows mean accuracy (y-axis) plotted against number of features (x-axis; from 1 feature to 7 features) for results from recursive elimination of features from 8 (which has accuracy of 0.5455 for patients with NSCLC treated with mono CPI and 0.4832 for chemotherapy patients). For the 7-gene feature set, the mean±standard deviation of the accuracy for CPI are 0.547±0.0136; for chemo are 0.491±0.0186. Error bars indicate the standard deviation of the accuracy from 5 random training/validation data splits. The seven-genes are: NF1, STK11, TSC2, STAG2, U2AF1, BRCA2, PDK1.

FIG. 14: Analysis Process

FIG. 15: Occurrence of diversity over Next Generations, as shown from freakgenie.com/heredity-and-evolution-mapping-our-genes-variation

FIG. 16A: Optimization Strategy, as shown at medium.com/@prassena.kannan/feature-extraction-using-response-code-on-customer-transaction-prediction-7d5826cca36c.

FIG. 16B: Optimization Strategy, as shown at hgl.com.

FIG. 17: Process.

FIG. 18: Drug Target Discovery with Genetic Algorithm.

FIG. 19A: Feature Selection Methods.

FIG. 19B: Additional Feature Selection Methods.

FIG. 20: Feature Selection Process.

FIG. 21: Drug Target Discovery with Genetic Algorithm.

FIG. 22: Robust solutions.

FIG. 23: Clustering Procedure: Feature Set PCA Visualization and KMeans Labels.

FIG. 24: Feature Set Performance.

FIG. 25: Feature “Popularity”.

FIG. 26: Linear Coefficient Strength of Features.

FIG. 27: Comparison to RFE feature results.

FIG. 28: Results using binary “is gene mutated” inputs.

FIG. 29: Further tuning possibilities.

### DETAILED DESCRIPTION OF THE INVENTION

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### Experimental Data

Lung cancer is the leading cause of cancer related mortality worldwide with NSCLC accounting for about 85% of all lung cancer histological subtypes^{1,2}. The discovery and FDA approval of check point inhibitors (CPI) completely revolutionized cancer therapy in a variety of malignancies, by achieving prolonged responses³⁻⁷. Unfortunately, despite the unprecedented prolonged response rates to CPIs the majority of patients are resistant to CPI therapy⁸. Resistance to CPIs can be categorized into two main patient groups: 1. Innate/primary resistant patient group, which never respond or derive clinical benefit from CPI therapy, and 2. Acquired resistance patient group, which initially respond to CPI therapy but eventually develop resistance and have disease progression⁸⁻¹⁰. Since the majority of patients treated with CPI fall into innate or acquired resistance group^{8,9,11}, there is an urgent and unmet need to understand CPI resistance mechanisms. The mechanistic understanding of CPI resistance will inevitably be followed by development of predictive biomarkers and potential targets for therapeutics discoveries aimed at reverting/preventing resistance to CPI therapy.

Extensive efforts are underway to identify predictive biomarkers utilizing various omics, histopathologic, clinical and computational approaches. These efforts led to the discovery that tumor mutational burden (TMB), microsatellite instability (MSI), PD-L1, JAK1/JAK2, IFNg, PTEN loss, PBRM1, STK11/KEAP1 mutations, antigen processing/presentation loss, WNT/b-catenin signaling can affect patient's response to CPI therapy¹². While the above biomarkers have led to important advances in the understanding of CPI resistance, the only approved biomarkers are TMB and PDL-1 levels¹⁰. However, even these approved biomarkers showed only moderate predictive value¹¹, and thus unfortunately, do not provide important mechanistic insight behind CPI resistance. In addition, several gene expression signatures were reported to be predictive of CPI response¹³⁻¹⁵, and while they increased our biological understanding behind CPI resistance, these signatures do not seem to generalize¹⁶ (at least in melanoma).

The limited genetic and clinical biomarkers to predict CPI response is a major bottle neck in developing novel therapeutics to target CPI resistance and in selection of biomarkers for patient selection.

### Methods

### Data Sources

The patent data set was obtained from the Flatiron Health de-identified Clinico-Genomic Database (CGDB) as available on Jan. 1, 2020 and which is described in Singal G, Miller P G, Agarwala V, et al. Association of Patient Characteristics and Tumor Genomics With Clinical Outcomes Among Patients With Non-Small Cell Lung Cancer Using a Clinicogenomic Database (CGDB). JAMA. 2019; 321 (14): 1391-1399. doi: 10.1001/jama.2019.3241. In particular, the de-identified Flatiron Health-Foundation Medicine NSCLC clinico-genomic database (FH-FMI CGDB). Patient treatment data between January 2011 and December 2019 (data collection cut-off date) were used for the analyses that follow.

### Defining Patient Treatment Outcome Groups

To better distinguish predictive (specific to CPI) from prognostic effects (independent of which treatment), we analysed standard-of-care chemotherapy cohorts. To better delineate the two effects, we also removed patients with early deaths (patients who died in the first 18 weeks after treatment start). This is because it has been reported that the CPI and chemotherapy survival curves did not differentiate until after about 18 weeks (Journal of Thoracic Oncology. 2018; 13:1156-1170)

Patients were categorized into two main outcome groups: durable-response and innate-resistance groups (FIG. 1B).

We used real-world progression (rwP; progressed or not on certain date) (Advances in Therapy, 2019; 36 (8): 2122-2136) and real-world response (rwR; CR, PR, SD or PD on certain date) (Advances in Therapy, 2021; 38:1843-1859) for defining the response groups.

A patient was considered to have “durable-response” if there was tumor response and no disease progression starting from 14 days after CPI treatment start to the end of the study duration. A patient was considered to have “innate-resistance” if there was disease progression without any tumor response during the study duration. To study the clinical benefit of CPI therapy and to have a more balanced number of patients in each response group, CR, PR and SD were considered as having tumor response from the rwR data. Having disease progression included rwP, death or a change to a non-CPI treatment line within the study duration. For study duration determination, sensitivity analysis using study durations ranging from 120 to 365 days, in ˜ 2-3 month increments, were performed. Study duration of 270 days resulted in an optimal balance in patient number in each response groups and is a clinically relevant duration. Disease progression within the first 14 days after CPI treatment was ignored, since it might not reflect the effect of the current treatment (recommendation by Flatiron Health).

### Treatment Data

Checkpoint inhibitor (CPI) analysis included monotherapies nivolumab, pembrolizumab, atezolizumab, durvalumab and avelumab. Chemotherapy patients from FH-FMI databases included patients with all the drugs annotated by Flatiron Health as “chemotherapy” who did not have “immunotherapy” in the patient's record in the database. For patients who had multiple lines of CPI or chemotherapy, their first CPI or chemotherapy records were used for analysis.

### TMB

Tumor mutation burden (TMB, number of mutations per Mb) calculated from targeted DNA sequencing using FoundationOne panel with solid baitsets (JAMA (2019) doi: 10.1001/jama.2019.3241) on tumor biopsies from all analysed patients was provided by FMI (Foundation Medicine Inc). TMB data from the most recent specimens collected before treatment start was used. Research Use Only (RUO) calculations based on FMI's research algorithm used at the time of collection were analysed (Genome Med. (2017) doi: 10.1186/s13073-017-0424-2.).

### Data Preparation

After associating a CPI resistance label to each patient, the mutations for each mono-CPI patient are filtered to remove synonymous mutations and then aggregated into a categorization: per-gene, as gain or loss of function per-gene, and as hotspots. There are 427 innate resistance patients and 372 durable response patients, with 284 mutations present when aggregated per-gene, 558 mutations present when aggregated as loss or gain of function per-gene, and 943 mutations when aggregated as hotspots. The input dataset is randomly split into training and test subsets, stratified by CPI resistance label, leaving 678 training patients and 121 test patients.

### Pathway Analysis Using CBDD

For pathway analysis the predictive genes were used as input to six different network-based algorithms implemented in CBDD R package, that utilizes the Metabase network and pathway data. The algorithms used were network propagation, interconnectivity, overconnectivity, hidden nodes, gene mania and causal reasoning. The top 100 nodes resulting from each algorithm were then used to run a pathway enrichment analysis on the Metabase pathways.

### Cohort Balancing; Baseline and Lab Value Bias Evaluation

In order to assess potential bias in the absence of explicit patient re-weighting, the number of correctly predicted patients in the test set categorized by baseline and lab values were compared with a Fisher's Exact Test3. For lab values, the median of the most recent 3 lab tests predating treatment within 1 year and up to 4 weeks after treatment start was used. Patients were divided into tertiles by the lab value measurements and the Fisher's test was applied comparing each pair of tertiles. After applying a False Discovery Rate correction4 no statistically significant bias in any of the baseline or lab value covariates was found (P<0.05), indicating that none of the models predict baseline or lab value quantities potentially correlated with CPI resistance rather than CPI resistance itself.

### Categorizing Mutations Using Binary, HFM/LFM and Hotspot Granular Categories

In binary classification, we consider any mutation within a particular gene as mutated (synonymous mutations were filtered) and genes without any mutations are considered WT. In HFM/LFM classification the following was flow was used to define the HFM and LFM categories: 1. We calculated the total number of amino acids mutated per gene 2. We calculated the frequency of mutations in each gene (I.e., how many patients had any mutation in that gene). 3. From #1 and #2 we calculated the average amino acid mutation rate (average amino acid mutation=gene level mutation frequency (#2)/Total amino acids mutated in the gene (#1). 4. HFM were assigned to any mutation that had 2× the average mutations per that specific amino acid and had more than/equal to 9 mutations in that gene. LFM was assigned to any mutation that had lower than 2× the average mutations per that specific amino acid and or had less than 9 mutations. Any mutation in the TERT promoter was classified as HFM. Amplifications were considered as HFM and deletions as LFM, the rationale behind this was that LFM tend to be loss of function and high Frequency mutations tend to be gain of function. In Hotspot granular classification, same as in HFM/LFM, but adding the amino acid mutation location to any HFM (TP53_178, meaning that HFM in TP53 in amino acid 178.

### Example 1—Genetic Algorithm Feature Selection

Genetic Algorithms (GA) can be adapted for use as a feature selection technique^{5,6}. In this study, we define a GA individual as a subset of the available input features of the dataset, represented as a binary mask over all features. The fitness of each individual is calculated based on the predictive power of a model which uses only the features contained in the subset and is trained to predict the binary CPI resistance category, ‘dura-response’ or ‘inn-resistance’.

Naive Bayes models with a Bernoulli prior are used. Naive Bayes models were chosen for the simplicity of their internal state, resistance to over-training, and interpretability. Random Forest and other ensemble based methods were attempted but found to require heavy hyperparameter tuning to avoid over-training during the genetic algorithm search. The Bernoulli prior is appropriate for binary input data and includes a penalty term for the feature not appearing, differentiating it from a multinomial prior.

For each GA individual, the fitness is the cross-validation (CV) accuracy score of a Naive Bayes model on the training set. The accuracy score is class-balanced to avoid favoring ‘dura-response’ over ‘inn-resistance’ or vice versa. The cross-validation uses stratification to keep the same fraction of ‘dura-response’ and ‘inn-resistance’ patients in each fold. The number of folds used was 5, a compromise to keep the number of patients in each fold high while keeping the number of folds high enough to be confident in the result. The training data is shuffled for each individual before cross validation to avoid overfitting on CV folds during GA optimization.

In each generation of the GA, individuals are selected for mutation or crossover using fitness proportionate selection⁷, which samples individuals probabilistically based on their fitness in order to maintain diversity in the GA breeding pool. The breeding pool of each generation is supplemented by a set of the highest fitness individuals from all previous generations. Mutation occurs by randomly removing or adding features to the subset while conserving the average number of features. The average number of features are conserved during mutation by partitioning the probability of mutation between adding features and removing features in order to retain (on average) the same number of features removed and added. Without this partition, mutation would tend to increase the size of models with less than 50% of the total features used and decrease the size of models with more than 50% of the features used regardless of the fitness of the result. Crossover occurs by randomly selecting each feature flag of the binary mask from two previous individuals and does not include further correction: crossover on average will produce offspring with the number of features halfway between each parent.

The GA procedure was run with the following parameters set. The GA is run for 200 generations each with a population of 1000 individuals. Larger populations and larger numbers of generations were not found to produce different results, as the GA was able to find an optima within this time. The first generation is generated randomly such that on average 10% of the features are included in each individual. This fraction was chosen to correspond roughly to the number of features at the end of the GA. During mutation, on average 1% of features are removed or added to an individual. The top 200 individuals over all generations are added to the breeding pool for each generation for a total breeding pool size of 1200 in each generation after the first. To generate each successive generation, 600 individuals are formed by mutating an individual from the breeding pool while the remaining 400 of each generation are formed by crossover of two previous individuals, a relative fraction chosen to slightly favor mutation in order to increase diversity in the population.

The GA is run 10 times for each mutational input categorization (binary per-gene, gain or loss of function, hotspot). Since each run of the GA tends to find separate local optima, these 10 runs along with the clustering technique described below are used to identify multiple local optima that are too distant for a single GA run to identify.

After 10 runs of 200 generations with a population size of 1000 there are 2,000,000 GA individuals which are available. The top 5% (100,000) of all individuals, based on their CV score, are selected and then clustered according to the similarity of the features they contain. The clustering is done using a simple KMeans clustering with 12 clusters to account for the expected 10 separate local optima (one from each run of the GA) plus some leeway for outliers.

In each cluster, the set of features which appear in more than 50% of cluster members is considered the characteristic set of features for that cluster. A final model is then trained on each of the 12 characteristic sets and evaluated on the test set.

### Feature Importance Calculation

In order to visualize the importance of individual mutations on the prediction outcomes, the internal state of the 36 Bernoulli Naive Bayes models were converted into their equivalent linear (logistic regression) coefficients⁸. This conversion is outlined below.

$$P ( M \vert C 0 ) = P ( mutation \vert class 0 )$$

This is the rate of mutation occurrence in the training set.

$$0 = log [ P ( M \vert C 0 ) 1 - P ( M \vert C 0 ) ]$$

This is the decision rule for Bernoulli Naive Bayes models for Class 0.

$$= 1 - 0 = log [ P ( M \vert C 1 ) ( 1 - P ( M \vert C 0 ) P ( M \vert C 0 ) ( 1 - P ( M \vert C 1 ) ]$$

When M>threshold, class 1 is predicted over class 0.

Notice that the value for each mutation is independent of all other mutations, therefore they are a feature of the training set itself. Bernoulli Naive Bayes models incorporate indirect mutation-mutation interactions through the model intercept/threshold (not derived here).

### Mutation Co-Occurrence Analysis

In order to assess potential relationships between small combinations of mutations and CPI resistance, a series of Fisher Exact Tests were performed on the co-occurrence of single mutations, mutation-pairs, and mutation-triples between the ‘dura-response’ and ‘inn-resistance’ CPI resistance groups.

For single mutations, the block diagram for the Fisher's Test was as below:

| | |
|---|---|
| ‘dura-response’ | ‘inn-resistance’ |

Mutation Present
Mutation Not Present

For pairs and triples of mutations, the block diagram was modified to remove the correlation between combinations of mutations containing the same mutation. For example, if mutation A is highly correlated to ‘dura-response’, then if it is paired with an uncorrelated mutation B, the pair A&B remains highly correlated to ‘dura-response’.

| | |
|---|---|
| ‘dura-response’ | ‘inn-resistance’ |

All Mutations Present
Any subset (other than
the full or empty set)
of Mutations Present

Using this corrected block diagram ensures that a significant single-mutation effect does not imply a significant mutational-pair effect and that each test is uncorrelated with each other and FDR corrections are appropriate. An additional requirement that each row/column sum of the block diagram must be at least 5 was applied to remove very rare mutational combinations.

For each of the mutation aggregations described above (binary gene, loss or gain of function, and granular hotspot) the mutation co-occurrence Fisher's Exact Test was computed and a False Discovery Rate correction applied.

### Example 2—Performance of 36 Signatures

While we identified several previously unreported mutations affecting CPI treatment response (e.g., NBN, PDGFRA, NF1, and the co-occurring mutations in TP53, KRAS and NF1), attempting to understand the biological mechanism(s) behind CPI resistance/response requires broadening the analysis beyond the aforementioned genes. Therefore, we investigated whether a mutation signature(s) (i.e., a collection of tumor mutations) can predict response to CPI. Since TMB is considered to be an established and important biomarker that correlates with response to CPI therapy, we used a TMB-only model as a benchmark of our future results. The TMB-only model trained on our NSCLC cohort showed an AuC score of 0.59 (FIG. 5C). Next, we utilized a machine learning (ML) approach which applied Naive Bayes models with a Bernoulli prior as the model architecture, embedded in a genetic algorithm (GA) for feature selection (FIG. 5A,) to reveal predictive mutational signatures. We used GA because of the large set of available features (Binary: 284, HFM/LFM: 558, Hotspot: 943) relative to the number of patients (n=799), as it can efficiently find the most predictive feature combinations (out of Binary: 2\hat{ }284, HFM/LFM: 2\hat{ }558, Hotspot: 2\hat{ }943) while including multi-feature interactions. Naive Bayes models were chosen for the simplicity of their internal state, resistance to over-training, and interpretability33. Analysis using our ML method on 678 patients (training set) resulted in 36 mutation signatures (12 for each input: binary, HFM/LFM and Hotspot) (Tables 1 to 3) with cross-validation AuC score ranges of 0.69-0.8 (Tables 8 to 10). In order to ensure generalizability and control overfitting, we held out 121 patients (test set) and used the 36 mutation signatures to predict CPI response. The generalizability analysis resulted in AUC score ranges of 0.55-0.64 (FIG. 5B, Tables 8 to 10), which supports the predictive power and generalizability of the mutation signatures. Importantly, we investigated the specificity of the mutation signatures on CPI response by utilizing the 36 mutation signatures to predict chemotherapy response in 304 chemo treated patients. Our results indicated that the average AuC scores for predicting response in chemo patients utilizing the 36 mutational signatures was 0.48±0.1, which is consistent with random chance, indicating that the mutational signatures are CPI response specific (FIG. 5B). Importantly, comparing the AuC scores between TMB-only and our binary mutational signature resulted in comparable results with AuC scores of 0.61 and 0.59 for binary and TMB respectively (FIG. 5C). These results are consistent with previously published AUC values for TMB34, 35 and provide support that our mutation signatures perform as well as the established TMB biomarker in prediction of CPI response. Altogether, our results indicate that our ML workflow performs as well as TMB, but provides the important advantage of interpretability, as the mutations deemed predictive can be explored to understand the biology behind response/resistance, which is not possible with a TMB-only prediction model.

### Example 3—Overlaps

Since each of the three ML inputs generated 12 separate predictive mutational signatures (Tables 1 to 3), we first confirmed that each mutation signature within the input is diverse (i.e., not large overlap between signatures) (FIG. 11) Next, we assessed feature (mutations) popularity across the 12 mutational signatures within each of the 3 ML inputs, as the most frequently selected mutations might indicate the importance of the mutation on CPI response (Table 4 and Table 5). We identified genes that were shared between the 12 independent mutational signatures within the 3 ML inputs sets (Table 5). We found that for the binary, HFM/LFM and Hotspot inputs, there was an overlap of 9, 13, 19 genes respectively within the 12 mutational signatures (Table 4). Among these overlaps, 8 mutations were shared across all 36 mutation signatures (FIG. 5D, Table 4). The overlap within each ML input across the 12 independent signatures suggests that the 9, 13, and 19 (Table below) genes are necessary in generating a predictive mutational signature and play an important role in CPI response.

| Input | Unique | Within input overlap | AVG |
|---|---|---|---|
| Binary | 58 | 9 | 24 |
| GOF/LOF | 149 | 13 | 52 |
| Hotspot | 165 | 19 | 87 |
| Input overlap | 39 | 8 | |

Moreover, the 8 shared genes (NF1, STK11, TSC2, BRCA2, BRAF, STAG2, U2AF1 and PDK1) shared by all mutation signatures represent a core set of mutations-arguably the most important—in predicting/affecting CPI response (FIG. 5D, Table 4). Next, we assessed the number of unique mutations selected as predictive within each of the 3 ML input across the 12 mutational signatures. Binary, HFM/LFM and hotspot mutational signatures contained 58, 149, and 165 unique features (genes/mutations), and had 39 genes overlapping between them (FIG. 5D, Table 5), further suggesting a core of mutations important for CPI response prediction. Altogether, our ML approach revealed a significant number of genes, many of which are previously unreported to play a role in immunotherapy response.

### Example 4—Relative Contributions of Individual Gene Mutations in Overall CPI Response

One of the advantages of using Naïve Bayes models with a Bernoulli prior is that it is a linear model (logistic regression) 36. This enables interpretability of the mutational signatures in two important ways: 1. enables us to quantify the relative contribution of each mutation to the prediction of CPI response (i.e., relative importance), and 2. allows us to associate each mutation with the specific response it predicts (i.e., does a mutation associate with durable-response or innate-resistance). Using the equivalent logistic regression formulation of the Naive Bayes models, we were able to assign each feature (mutation) to a corresponding CPI response group with a numeric contribution, allowing us to sort the mutations by their contribution/importance to CPI response (FIG. 6A, FIG. 12, Table 6 and Table 7). In Binary mutational signatures we found that TSC2 mutations were the top contributor to durable-response and GID4 was the top contributor to innate-resistance (FIG. 6A, Table 6 and Table 7). In HFM/LFM mutational signatures we found that FGF19 LFM mutations were the top contributor to durable response and MAP3K1 HFM mutations were the top contributor to innate resistance (FIG. 6A, Table 6 and Table 7). In Hotspot mutational signatures we found that FGF19 LFM was the top contributor to durable-response and EGFR mutation at amino acid 746 was the top contributor to innate-resistance. Interestingly, we observed a set of genes that had opposite effects on CPI response, depending on the type of mutation (FIG. 6B, Table 6 and Table 7). For example, in HFM/LFM input, we found that HFM mutations in PDGFRB were associated with durable-response, while LFM mutations were associated with innate-resistance (FIG. 6B). Furthermore, in Hotspot inputs, we found that certain TP53 HFM mutations were associated with durable-response, while other TP53 HFM mutations were associated with innate-resistance (FIG. 6B). Altogether these results provide a previously unreported link between certain mutations and CPI response, and revealed that mutations within the same gene can lead to opposite responses.

### Example 5—Pathways

While it is important to identify individual mutations that affect CPI response, there is an unmet need to understand the biology/biological-processes behind these mutations and how they affect CPI response. In order to shed some light on the biological processes behind the aforementioned predictive mutation signatures, we investigated if these signatures fall into meaningful biological process. As traditional pathway enrichment analysis looks at biological pathways as a collection of genes, we wanted to apply network/topology-based pathway analysis that takes gene-gene (or protein-protein) interactions into account when performing pathway analysis. To that end, we used 8 different algorithms to perform topology assisted pathway analysis, utilizing the Computational Biology Methods for Drug Discovery (CBDD) R package37 developed by Clarivate. This approach provides the benefit of discovering additional pathways that would otherwise not be detected in an enrichment only approach. Network/topology pathway analysis for binary input revealed that the top 10 pathways across 8 algorithms were associated with cell cycle, IL-6, DNA damage response/repair (DDR), PDGF, Leptin, and IFN alpha/beta signaling (FIG. 7). For HFM/LFM input the top 10 pathways were associated with epigenetic changes, YAP/TAZ, IL-6, DDR, PDGF, leptin and cell cycle related signaling. Since there is overlap of 39 genes between the three ML inputs (binary, HFM/LFM, hotspot), we checked which pathways are enriched in this overlap. Network/Topology based pathway analysis of the 39 gene overlap between the three inputs revealed that the top 10 pathways were associated with cell cycle, ESR1, PDGF, IL-6, DDR, IFN-alpha/beta, and EGFR signaling (FIG. 7). We chose not to preform pathway analysis on the Hotspot granular mutation signature as it included 58% of possible genes, and can potentially bias our analysis. Altogether, our pathway analysis reveals several previously reported (e.g., IL-6, IFN-alpha/beta, DDR) and unreported tumor intrinsic pathways that may be involved in CPI response (e.g., YAP/TAZ, leptin and PDGF signaling)

### Example 6—IL-6 Signalling

Since IL-6 signaling appeared in multiple pathway analysis results, we investigated its importance in NSCLC patients treated with Atezolizumab. We found that serum levels of IL-6 were elevated in both stable disease (SD) and progressive disease (PD) patients when compared to partial response (PR) patients in both Response evaluation criteria in solid tumors (RECIST) and immune-related response criteria (irRC). Furthermore, OS analysis revealed a significant survival increase in patients with lower IL-6 serum levels in NSCLC patients treated with Atezolizumab, which is consistent with previous reports. Altogether, the confirmation of the involvement of IL-6 in resistance mechanism supports the validity of our ML pipeline, which allows further insight into the biological pathways and mechanism(s) behind CPI resistance/response.

### Example 7—Recursive Elimination of Features

In order to assess the effect of reducing the number of features (i.e. genes) on the predictive accuracy of classification, a recursive elimination strategy was adopted. As shown in FIG. 13, recursive elimination of features from 8 (which has accuracy of 0.5455 for patients with NSCLC treated with mono CPI and 0.4832 for chemotherapy patients) was conducted to assess performance (mean accuracy) of 7-gene, 6-gene, 5-gene, 4-gene, 3-gene, 2-gene and 1-gene models. For the 7-gene feature set, the mean±standard deviation (sd) of the accuracy for CPI are 0.547±0.0136; for chemotherapy are 0.491±0.0186. Error bars indicate the standard deviation of the accuracy from 5 random training/validation data splits. The seven-genes are: NF1, STK11, TSC2, STAG2, U2AF1, BRCA2, PDK1. Without wishing to be bound by any particular theory, it is presently believed that reduction below 5 features (i.e. the 4-gene and below models) exhibit notably decreased mean accuracy. Therefore, models involving 5 features or greater may be chosen for their improved accuracy. The comparison between accuracy of prediction of CPI response vs. that of chemotherapy response evidences the specific nature of the CPI response predictive models as disclosed herein.

### Example 8—CGDB Subset of Minimal Genes for Predicting Durable Response Vs. Innate Resistance

The present inventors conducted further analysis to determine optimized minimal gene sets that maintain reasonable predictive performance and below which predictive performance is negatively impacted. This led to the following feature (gene) sets, each of which exhibited performance (mean accuracy) in the present data set that was comparable to other feature sets described herein, including feature sets involving larger number of genes and/or mutations.
Binary gene input (10 gene set): BRAF, BRIP1, STK11, CDK12, CTNNA1, FAS, NRAS, NOTCH3, PIK3CA and RAD51C.
HFM/LFM (GOF/LoF) (15 gene set): PBRM1 LOF, BRIP1 LOF, PTEN LOF, CDKN2A LOF, STK11 GOF, CDKN2B LOF, U2AF1 GOF, CTNNA1 LOF, FGF10 GOF, FGF19 LOF, AKT2 GOF, NBN LOF, ALOX12B LOF, BRAF GOF and NF1 GOF.
Hotspot (8 gene set): BRIP1_LOF, CDKN2B_LOF, U2AF1 GOF 34, CTNNA1 LOF, ALOX12B LOF, EGFR GOF 746, FAS LOF and KMT2A LOF.

Furthermore, the present inventors have tested a set of 5 genes selected with prior knowledge and achieved 57% AUC (prediction performance), and without those 5 genes, accuracy drops ˜3% from using all features.

The two lists of 5 genes are shown below. The predictive performance of each was approximately the same.
First 5-gene set: STK11, BRAF, BRIP1, U2AF1 and NF1.
Second 5-gene set: STK11, PDGFRA, BRAF, BRIP1 and CTNNA1.

### ANNEX 1—Tables

TABLE 1
Cluster information for binary mutations
Binary

| cluster1 | cluster2 | cluster3 | cluster4 | cluster5 | cluster6 |
|---|---|---|---|---|---|
| NF1 | NF1 | NF1 | NF1 | STK11 | STK11 |
| STK11 | STK11 | STK11 | STK11 | NF1 | TSC2 |
| TSC2 | TSC2 | BRAF | TSC2 | TSC2 | BRAF |
| ATR | TP53 | TSC2 | STAG2 | BRAF | ATR |
| STAG2 | BRCA2 | ATR | BRAF | STAG2 | NBN |
| BRAF | BRAF | NBN | NBN | ATR | NF1 |
| U2AF1 | ATRX | STAG2 | ATR | PDGFRA | PDGFRA |
| ATRX | STAG2 | BRCA2 | BRCA2 | U2AF1 | U2AF1 |
| BRCA2 | FGF23 | U2AF1 | U2AF1 | BRCA2 | BRCA2 |
| TET2 | U2AF1 | PDGFRB | GID4 | NBN | STAG2 |
| GID4 | TET2 | FGF23 | PDK1 | PDK1 | PDK1 |
| RARA | POLE | PDK1 | FGF23 | GID4 | TET2 |
| PDK1 | BRIP1 | GID4 | IDH1 | CCNE1 | GID4 |
| NBN | PDK1 | PDGFRA | PPP2R1A | CDK4 | FOXL2 |
| FOXL2 | ATR | CD79A | JUN | FOXL2 | CDK4 |
| IDH1 | ASXL1 | CCNE1 | ATRX | PDGFRB | PPP2R1A |
| CTNNA1 | ERCC4 | FOXL2 | FH | MLH1 | CCNE1 |
| FGF23 | PAX5 | TET2 | CD79A | CD79A | CSF1R |
| MPL | CTNNA1 | CTNNA1 | ASXL1 | FGF23 | ASXL1 |
| AKT1 | MPL | JUN | CTNNA1 | RAD51 | CTNNA1 |
| CCNE1 | GID4 | RAD51 | TET2 | IDH1 | RAD51 |
| FANCL | CD79A | IDH1 | PDCD1LG2 | CSF1R | IDH1 |
| ERCC4 | TSC1 | CDK4 | FOXL2 | | FGF23 |
| SMARCA4 | MAP2K1 | CSF1R | MDM2 | | CDK6 |
| | NRAS | NKX2-1 | MEF2B | | FUBP1 |
| | RARA | | CCNE1 | | AKT1 |
| | PDCD1LG2 | | PDGFRB | | FGFR1 |
| | | | CDK4 | | NTRK2 |
| | | | | | MLH1 |
| cluster7 | cluster8 | cluster9 | cluster10 | cluster11 | cluster12 |
| ATR | STK11 | NF1 | NF1 | STK11 | STK11 |
| NF1 | TSC2 | STK11 | STK11 | NF1 | NF1 |
| STK11 | BRAF | TP53 | TSC2 | TSC2 | TSC2 |
| TSC2 | BRCA2 | TSC2 | ASXL1 | TP53 | STAG2 |
| STAG2 | PDGFRA | BRCA2 | ATR | POLE | ATR |
| BRCA2 | NF1 | BRAF | BRAF | BRCA2 | ATRX |
| NBN | U2AF1 | U2AF1 | BRCA2 | ATRX | BRAF |
| BRAF | MLH1 | ATR | U2AF1 | BRAF | ASXL1 |
| PDK1 | ASXL1 | ATRX | CDK4 | STAG2 | U2AF1 |
| U2AF1 | POLE | BRIP1 | RARA | TET2 | BRCA2 |
| TET2 | PDK1 | SMAD4 | CTNNA1 | U2AF1 | TET2 |
| IDH1 | RARA | ASXL1 | STAG2 | FGF23 | PDK1 |
| GID4 | STAG2 | IDH1 | PDK1 | BRIP1 | GID4 |
| CD79A | BRIP1 | STAG2 | CCNE1 | ASXL1 | MPL |
| CTNNA1 | CDK4 | FGF23 | MLH1 | PDK1 | IDH1 |
| FGF23 | PDCD1LG2 | TET2 | CDKN1A | ERCC4 | CTNNA1 |
| CCNE1 | FANCG | GID4 | PPP2R1A | ATR | PPP2R1A |
| AKT1 | CCNE1 | CTNNA1 | MPL | PAX5 | RARA |
| ATRX | TET2 | PDK1 | ATRX | CTNNA1 | NBN |
| ASXL1 | ATR | PDCD1LG2 | RAD51B | TSC1 | CDK4 |
| PDGFRB | PPP2R1A | QKI | ERCC4 | MPL | TSC1 |
| JUN | FUBP1 | FOXL2 | IDH1 | PDCD1LG2 | MEF2B |
| | | MPL | AKT1 | RARA | FGF23 |
| | | CD79A | POLE | BRCA1 | CEBPA |
| | | TSC1 | CEBPA | CEBPA | CDK6 |
| | | MEF2B | MEF2B | CD79A | |
| | | FUBP1 | PDCD1LG2 | GID4 | |
| | | CCNE1 | GID4 | CDK4 | |
| | | BRD4 | | MAP2K1 | |
| | | | | NRAS | |
| | | | | SMAD4 | |
| | | | | MEF2B | |

TABLE 2
Cluster information for GOF/LOF mutations.
GOF/LOF

| cluter1 | cluster2 | cluster3 | cluster4 | cluster5 | cluster6 |
|---|---|---|---|---|---|
| NF1_LOF | NF1_LOF | NF1_LOF | NF1_LOF | NF1_LOF | NF1_LOF |
| STK11_LOF | STK11_LOF | STK11_LOF | STK11_LOF | STK11_LOF | STK11_LOF |
| KMT2A_LOF | BRAF_GOF | BRAF_GOF | FGF19_LOF | ASXL1_LOF | BRAF_GOF |
| ASXL1_LOF | ASXL1_LOF | TSC2_LOF | BRAF_GOF | BRAF_GOF | TSC2_LOF |
| FGF19_LOF | BRCA2_LOF | ATRX_LOF | STK11_GOF | ATRX_LOF | STAG2_LOF |
| TSC2_LOF | TSC2_LOF | BRCA2_LOF | ATRX_LOF | FGF19_LOF | FGF19_LOF |
| BRAF_GOF | STAG2_LOF | FGF19_LOF | BRCA2_LOF | STK11_GOF | PDK1_LOF |
| IDH1_GOF | NKX2-1_LOF | STAG2_LOF | TSC2_LOF | TSC2_LOF | ASXL1_LOF |
| STAG2_LOF | ATR_LOF | ASXL1_LOF | NFKBIA_GOF | BRCA2_LOF | STK11_GOF |
| STK11_GOF | FGF19_LOF | PDK1_LOF | ASXL1_LOF | KMT2A_LOF | PDGFRA_LOF |
| BRCA2_LOF | PDK1_LOF | STK11_GOF | CDK4_GOF | STAG2_LOF | IDH1_GOF |
| PAX5_GOF | KMT2A_LOF | PDGFRA_LOF | JAK1_GOF | NKX2-1_LOF | ATRX_LOF |
| SOX2_LOF | NFKBIA_GOF | RB1_GOF | JUN_LOF | PDK1_LOF | U2AF1_GOF |
| PDK1_LOF | STK11_GOF | NKX2-1_LOF | IDH1_GOF | IDH1_GOF | NKX2-1_LOF |
| RB1_GOF | RB1_GOF | IDH1_GOF | PDK1_LOF | RB1_GOF | NBN_LOF |
| ATRX_LOF | PDGFRA_LOF | CDK4_GOF | KMT2A_LOF | FANCG_GOF | PPP2R1A_GOF |
| PDCD1LG2_GOF | U2AF1_GOF | U2AF1_GOF | STAG2_LOF | RAD52_GOF | NRAS_LOF |
| GATA3_GOF | IDH1_GOF | NFKBIA_GOF | NKX2-1_LOF | CDK4_GOF | PIK3C2G_GOF |
| U2AF1_GOF | CEBPA_LOF | JUN_LOF | PPP2R1A_GOF | ATR_LOF | JUN_LOF |
| CTNNA1_LOF | PPP2R1A_GOF | PBRM1_LOF | PAX5_GOF | MUTYH_GOF | JAK1_GOF |
| NKX2-1_LOF | PAX5_GOF | QKI_LOF | U2AF1_GOF | U2AF1_GOF | NTRK2_LOF |
| CEBPA_LOF | AKT3_GOF | GATA3_GOF | PIK3C2G_GOF | PPP2R1A_GOF | ACVR1B_LOF |
| PIK3C2G_GOF | PIK3C2G_GOF | JAK1_GOF | FGF23_LOF | MAP3K1_LOF | BRCA2_GOF |
| PPP2R1A_GOF | VEGFA_LOF | PIK3C2G_GOF | ZNF703_GOF | NRAS_LOF | CEBPA_LOF |
| HSD3B1_GOF | FGF14_GOF | MYC_LOF | ATR_LOF | VEGFA_LOF | FANCL_LOF |
| JAK1_GOF | GATA6_GOF | EZH2_LOF | CDH1_LOF | CDKN2B_LOF | BRD4_GOF |
| PBRM1_LOF | SOX2_LOF | MYD88_GOF | PBRM1_LOF | EZH2_GOF | RB1_GOF |
| GATA6_GOF | MYCN_LOF | KMT2A_LOF | MAP3K1_LOF | TNFAIP3_GOF | FGF23_LOF |
| NFKBIA_GOF | HGF_LOF | FGF14_GOF | CCNE1_GOF | CREBBP_GOF | REL_LOF |
| VEGFA_LOF | LTK_GOF | CD79A_LOF | CBFB_LOF | DAXX_GOF | PDCD1LG2_GOF |
| JUN_LOF | PTPN11_LOF | PPP2R1A_GOF | FGFR4_GOF | CDK6_GOF | VEGFA_LOF |
| FGF14_GOF | NTRK2_LOF | PAX5_GOF | PPARG_LOF | BRCA2_GOF | MAP3K1_GOF |
| CDK6_GOF | FH_GOF | CEBPA_LOF | KEAP1_GOF | PDCD1LG2_GOF | RNF43_GOF |
| CD274_LOF | CD79A_LOF | SDHC_LOF | MYCN_LOF | GATA6_GOF | MYCN_LOF |
| KLHL6_GOF | PRKAR1A_GOF | CD274_GOF | AURKA_LOF | FLT3_GOF | QKI_LOF |
| SYK_LOF | MEF2B_GOF | MAP3K1_GOF | GATA3_GOF | CHEK1_GOF | TGFBR2_GOF |
| BRCA2_GOF | PARP1_GOF | ZNF703_GOF | IRF4_GOF | ACVR1B_LOF | BRCA2_LOF |
| CUL4A_LOF | PDCD1LG2_GOF | CD79B_GOF | RB1_GOF | DNMT3A_GOF | CD274_GOF |
| GABRA6_GOF | SETD2_GOF | NBN_LOF | BRCA2_GOF | FGFR2_GOF | KLHL6_GOF |
| ACVR1B_LOF | GNAQ_GOF | KEAP1_GOF | TGFBR2_GOF | AKT3_GOF | HGF_LOF |
| BCL2L1_LOF | CHEK1_GOF | MAP2K2_GOF | NRAS_LOF | CUL3_GOF | BCORL1_GOF |
| MYD88_GOF | ERCC4_LOF | GRM3_GOF | GATA6_GOF | CEBPA_LOF | GATA4_GOF |
| TGFBR2_GOF | ABL1_GOF | VEGFA_LOF | FANCA_LOF | H3F3A_GOF | PDGERB_LOF |
| RAD51_LOF | CUL4A_LOF | CUL3_GOF | CDKN2B_LOF | POLD1_GOF | RARA_GOF |
| ERCC4_LOF | FUBP1_LOF | SYK_LOF | FLCN_GOF | BAP1_LOF | TNFAIP3_GOF |
| RAC1_LOF | CDK6_GOF | NRAS_LOF | ACVR1B_LOF | CTNNA1_LOF | BRIP1_GOF |
| AKT1_GOF | TGFBR2_GOF | SUFU_LOF | MYC_LOF | CD79A_LOF | CDK6_GOF |
| MAP3K1_GOF | PTEN_GOF | AKT2_GOF | RPTOR_GOF | NOTCH3_GOF | PAX5_GOF |
| PARP1_GOF | JAK1_GOF | MED12_GOF | NBN_LOF | PDGFRB_GOF | PDCD1LG2_LOF |
| MYC_LOF | SDHC_LOF | CREBBP_GOF | ARID1A_GOF | | CDK4_GOF |
| RAD51C_GOF | RAC1_LOF | BCORL1_GOF | AKT1_LOF | | MAP2K4_GOF |
| FGF23_LOF | FGFR4_GOF | SMARCA4_GOF | CEBPA_LOF | | |
| EZH2_GOF | | | CD274_GOF | | |
| AKT1_LOF | | | CUL3_GOF | | |
| PRKAR1A_GOF | | | MTOR_GOF | | |
| NF2_GOF | | | FLT3_GOF | | |
| FGF23_GOF | | | MUTYH_LOF | | |
| CDKN2B_LOF | | | CHEK2_LOF | | |
| ABL1_GOF | | | CDKN2A_LOF | | |
| CHEK1_LOF | | | RARA_LOF | | |
| SDHC_GOF | | | | | |
| cluster7 | cluter8 | cluster9 | cluster10 | cluster11 | cluster12 |
| NF1_LOF | NF1_LOF | NF1_LOF | NF1_LOF | NF1_LOF | NF1_LOF |
| STK11_LOF | BRAF_GOF | STK11_LOF | STK11_LOF | STK11_LOF | STK11_LOF |
| BRAF_GOF | STK11_LOF | BRAF_GOF | BRAF_GOF | STK11_GOF | ASXL1_LOF |
| ASXL1_LOF | ASXL1_LOF | BRCA2_LOF | ATR_LOF | ASXL1_LOF | BRAF_GOF |
| BRCA2_LOF | TSC2_LOF | ASXL1_LOF | KMT2A_LOF | BRCA2_LOF | FGF19_LOF |
| ATRX_LOF | STAG2_LOF | KMT2A_LOF | TSC2_LOF | BRAF_GOF | STAG2_LOF |
| PAX5_GOF | ATRX_LOF | NKX2-1_LOF | ASXL1_LOF | PBRM1_LOF | KMT2A_LOF |
| FGF19_LOF | NKX2-1_LOF | TSC2_LOF | PDK1_LOF | STAG2_LOF | TSC2_LOF |
| KMT2A_LOF | STK11_GOF | PDK1_LOF | FGF19_LOF | FGF19_LOF | IDH1_GOF |
| TSC2_LOF | BRCA2_LOF | STAG2_LOF | NKX2-1_LOF | RB1_GOF | BRCA2_LOF |
| PDK1_LOF | FGF19_LOF | ATR_LOF | SOX2_LOF | IDH1_GOF | STK11_GOF |
| IDH1_GOF | ATR_LOF | PDGFRA_LOF | IDH1_GOF | PDK1_LOF | PDK1_LOF |
| STK11_GOF | PDGFRA_LOF | FGF19_LOF | BRCA2_LOF | CTNNA1_LOF | RB1_GOF |
| NKX2-1_LOF | PDK1_LOF | PIK3C2G_GOF | CEBPA_LOF | U2AF1_GOF | PAX5_GOF |
| ATR_LOF | RB1_GOF | NFKBIA_GOF | PPP2R1A_GOF | PPP2R1A_GOF | PIK3C2G_GOF |
| STAG2_LOF | IDH1_GOF | STK11_GOF | FGF23_LOF | JUN_LOF | PDCD1LG2_GOF |
| U2AF1_GOF | NFKBIA_GOF | RB1_GOF | PDCD1LG2_GOF | NKX2-1_LOF | ATRX_LOF |
| PIK3CA_GOF | U2AF1_GOF | VEGFA_LOF | KLHL6_GOF | TSC2_LOF | SOX2_LOF |
| JAK1_GOF | PPP2R1A_GOF | PAX5_GOF | SYK_LOF | ERRFI1_LOF | U2AF1_GOF |
| KLHL6_GOF | GATA3_GOF | CEBPA_LOF | NBN_LOF | ATRX_LOF | CEBPA_LOF |
| MAP3K13_GOF | JAK1_GOF | PPP2R1A_GOF | STAG2_LOF | PIK3C2G_GOF | NKX2-1_LOF |
| PIK3C2G_GOF | JUN_LOF | MYCN_LOF | NFKBIA_GOF | CHEK2_LOF | PPP2R1A_GOF |
| NBN_LOF | ERCC4_LOF | U2AF1_GOF | GATA6_GOF | XRCC2_LOF | GATA3_GOF |
| CDK4_GOF | FGF14_GOF | AKT3_GOF | U2AF1_GOF | PDCD1LG2_GOF | NFKBIA_GOF |
| HGF_LOF | IRF4_GOF | GATA6_GOF | GNA13_LOF | RNF43_GOF | HSD3B1_GOF |
| RAD51C_LOF | CDH1_LOF | IDH1_GOF | PAX5_GOF | SOX2_LOF | CTNNA1_LOF |
| NRAS_LOF | HGF_LOF | FGF14_GOF | MAP3K1_GOF | JAK1_GOF | JAK1_GOF |
| CDK6_GOF | ZNF703_GOF | SOX2_LOF | KDM6A_GOF | PAX5_GOF | CD274_LOF |
| MDM2_LOF | MAP3K1_GOF | HGF_LOF | GATA3_GOF | BRCA2_GOF | GATA6_GOF |
| FH_GOF | MYCN_LOF | ERCC4_LOF | PARP1_GOF | CDKN1A_GOF | PBRM1_LOF |
| PPP2R1A_GOF | JUN_GOF | PTPN11_LOF | RARA_GOF | MYD88_GOF | KLHL6_GOF |
| GNA13_LOF | VEGFA_LOF | NTRK2_LOF | HGF_LOF | FGF23_LOF | CDK6_GOF |
| AXL_GOF | BRCA2_GOF | FUBP1_LOF | PDGFRB_GOF | QKI_LOF | SYK_LOF |
| RB1_GOF | FANCG_GOF | PARP1_GOF | JAK1_GOF | GATA3_GOF | FGF14_GOF |
| CTNNA1_LOF | ACVR1B_LOF | GNAQ_GOF | MYCN_LOF | BCL2L1_LOF | AKT1_GOF |
| CCND2_LOF | PIK3C2G_GOF | FH_GOF | NOTCH2_GOF | RARA_GOF | JUN_LOF |
| GATA3_GOF | BARD1_GOF | PRKAR1A_GOF | RAD51C_GOF | AKT2_GOF | MYC_LOF |
| TGFBR2_GOF | CEBPA_LOF | LTK_GOF | TGFBR2_GOF | VEGFA_LOF | TGFBR2_GOF |
| TSC2_GOF | NBN_LOF | MEF2B_GOF | RAD52_GOF | MAP3K13_GOF | CUL4A_LOF |
| MYC_LOF | MPL_GOF | TGFBR2_GOF | VEGFA_LOF | HSD3B1_GOF | BRCA2_GOF |
| RAD54L_LOF | PBRM1_LOF | JAK1_GOF | RBM10_GOF | AKT3_GOF | RAC1_LOF |
| VEGFA_LOF | QKI_LOF | RAD51C_LOF | CUL3_GOF | CCNE1_GOF | MYD88_GOF |
| CCNE1_GOF | FGFR4_GOF | CHEK1_GOF | RAC1_LOF | BRD4_GOF | GABRA6_GOF |
| CD274_GOF | CHEK1_GOF | SETD2_GOF | ERBB2_GOF | PIK3R1_GOF | MAP3K1_GOF |
| MAP3K1_GOF | | ABL1_GOF | PIK3CB_LOF | FGF6_GOF | VEGFA_LOF |
| SOX2_LOF | | PTEN_GOF | XRCC2_LOF | MYC_LOF | BCL2L1_LOF |
| GATA6_GOF | | CUL4A_LOF | CTNNA1_GOF | ABL1_GOF | ACVR1B_LOF |
| ARFRP1_LOF | | IRF2_GOF | LTK_GOF | GABRA6_GOF | PRKAR1A_GOF |
| DAXX_GOF | | BTG1_GOF | NRAS_LOF | PDCD1LG2_LOF | ABL1_GOF |
| SDHD_GOF | | CD79A_LOF | CBFB_GOF | STAG2_GOF | PARP1_GOF |
| PDGFRB_GOF | | WT1_LOF | IRF4_GOF | | CUL3_GOF |
| CTNNA1_GOF | | PDCD1LG2_GOF | JUN_LOF | | FGFR2_GOF |
| TSC1_GOF | | HSD3B1_LOF | FBXW7_GOF | | RAD51_LOF |
| | | | FGF14_GOF | | RAD51C_GOF |

TABLE 3
Cluster information for hotspot mutations.
Hotspot

| cluster1 | cluster2 | cluster3 |
|---|---|---|
| NF1_LOF | NF1_LOF | NF1_LOF |
| STK11_LOF | TSC2_LOF | KMT2A_LOF |
| ASXL1_LOF | KMT2A_LOF | BRAF_GOF_600 |
| KMT2A_LOF | ASXL1_LOF | STK11_LOF |
| PDGFRA_LOF | BRAF_GOF_600 | ATR_LOF |
| ATR_LOF | STK11_LOF | TSC2_LOF |
| NFKBIA_GOF | ATR_LOF | DNMT3A_GOF_882 |
| BRAF_GOF_600 | PDGFRA_LOF | FGF19_LOF |
| TSC2_LOF | BRCA2_LOF | ASXL1_LOF |
| FGF19_LOF | STAG2_LOF | BRCA2_LOF |
| MYCN_LOF | NKX2-1_LOF | PDGFRA_LOF |
| NKX2-1_LOF | FGF19_LOF | CDKN2A_GOF_151 |
| BRCA2_LOF | NFKBIA_GOF | WT1_LOF |
| CDKN2A_GOF_151 | CDKN2A_GOF_151 | NFKBIA_GOF |
| PDK1_LOF | NFE2L2_GOF_24 | NKX2-1_LOF |
| TP53_GOF_331 | TP53_GOF_282 | MYCN_LOF |
| TP53_GOF_282 | PDK1_LOF | PDK1_LOF |
| WT1_LOF | GATA3_GOF | TP53_GOF_331 |
| QKI_LOF | PAX5_GOF | CDC73_GOF |
| NFE2L2_GOF_24 | DNMT3A_GOF_882 | PPP2R1A_GOF |
| VEGFA_LOF | U2AF1_GOF_34 | STAG2_LOF |
| U2AF1_GOF_34 | TP53_GOF_331 | U2AF1_GOF_34 |
| EGER_GOF_719 | VEGFA_LOF | VEGFA_LOF |
| PPP2R1A_GOF | TP53_GOF_272 | TP53_GOF_N82 |
| DNMT3A_GOF_882 | CEBPA_LOF | NFE2L2_GOF_77 |
| STAG2_LOF | PTCH1_GOF_48 | NFE2L2_GOF_24 |
| GATA3_GOF | STK11_GOF_291 | CCNE1_GOF |
| HGF_LOF | DIS3_GOF_458 | FANCG_GOF |
| PIK3CA_GOF_1043 | TP53_GOF_285 | TP53_GOF_560 |
| PIK3C2G_GOF_1088 | STK11_GOF_37 | EGFR_GOF_719 |
| TP53_GOF_159 | AKT3_GOF | STK11_GOF_291 |
| TP53_GOF_272 | TP53_GOF_244 | EGFR_GOF_858 |
| MTOR_GOF_1834 | PIK3C2G_GOF_1088 | BRAF_GOF_466 |
| MYCN_GOF | TP53_GOF_920 | HGF_LOF |
| NRAS_LOF | PARP1_GOF | EGER_GOF_746 |
| STK11_GOF_291 | KEAP1_GOF_116 | TP53_GOF_159 |
| JUN_LOF | KDM5A_GOF | KEAP1_GOF_260 |
| AR_GOF_493 | MAP3K1_GOF | PIK3C2G_GOF_1088 |
| CEBPA_LOF | JUN_LOF | JUN_LOF |
| ERBB2_GOF_755 | KEAP1_GOF_272 | ZNF703_GOF |
| RB1_GOF | GNAQ_GOF | AR_GOF_69 |
| BRAF_GOF_466 | POLD1_GOF | GATA3_GOF |
| STK11_GOF_221 | IDH1_GOF | KDM5C_GOF_1546 |
| PTEN_GOF_59 | CD274_GOF | KEAP1_GOF_234 |
| AKT3_GOF | FH_GOF | BRCA2_GOF |
| NFE2L2_GOF_77 | STK11_GOF_84 | PIK3CA_GOF_1043 |
| STK11_GOF_251 | MYCN_LOF | TP53_GOF_195 |
| SF3B1_GOF_666 | CDK8_GOF | KEAP1_GOF_332 |
| ARID1A_GOF_21 | PARP3_GOF | STK11_GOF_84 |
| KEAP1_GOF | CDKN2A_GOF_80 | QKI_LOF |
| CUL4A_LOF | TNFAIP3_GOF | SMARCB1_GOF |
| KEAP1_GOF_364 | MYD88_GOF_265 | KEAP1_GOF_364 |
| MAP2K1_GOF_102 | KEAP1_GOF_483 | KLHL6_GOF |
| ALOX12B_GOF | TSC1_GOF | CEBPA_LOF |
| MYD88_GOF_265 | FGF14_GOF | STK11_GOF_163 |
| ACVR1B_LOF | BRCA2_GOF | FBXW7_GOF_505 |
| SMARCA4_GOF_1160 | SF3B1_GOF_666 | AKT1_LOF |
| TNFRSF14_LOF | CCNE1_GOF | MET_GOF_3028 |
| TP53_GOF_278 | PPP2R1A_GOF | MYCN_GOF |
| RAD51B_LOF | STK11_GOF_308 | SMARCA4_GOF_1162 |
| FLT1_GOF | EGFR_GOF_790 | PIK3CA_GOF_345 |
| KEAP1_GOF_493 | CDKN1A_GOF | CD79A_LOF |
| MDM2_LOF | TP53_GOF_298 | PPARG_GOF |
| PIK3R1_LOF | CDKN2A_GOF_61 | XRCC2_LOF |
| AR_GOF_468 | SOX2_LOF | AR_GOF_70 |
| SMAD4_GOF_351 | EGFR_GOF_19 | MYD88_GOF_265 |
| POLE_GOF | MAP3K1_GOF_949 | IRF2_GOF |
| CTNNB1_GOF_33 | PALB2_GOF | RAD52_GOF |
| NBN_GOF_680 | IRF4_GOF | PIK3CB_GOF |
| CDK4_GOF | STK11_GOF_57 | PIK3CA_GOF_1047 |
| KEAP1_GOF_417 | KEAP1_GOF_470 | AR_GOF_465 |
| KEAP1_GOF_116 | CHEK2_GOF_392 | TP53_GOF_278 |
| ATM_GOF_337 | MAP3K1_GOF_5 | AMER1_GOF_385 |
| SDHA_GOF_457 | KEAP1_GOF_153 | ERBB2_GOF_755 |
| GATA4_GOF | ERBB2_GOF_755 | MSH6_GOF_1088 |
| PDGFRB_GOF | CBFB_LOF | HSD3B1_GOF_75 |
| FBXW7_GOF_479 | KEAP1_GOF_260 | CHEK1_GOF |
| CTNNB1_GOF_41 | FGFR4_GOF | KEAP1_GOF_244 |
| CDKN1A_GOF | GNAS_GOF_407 | CDKN1A_GOF |
| ZNF217_GOF_410 | SDHB_LOF | CDKN2A_GOF_69 |
| BRAF_GOF_464 | PIK3CB_LOF | TP53_GOF_272 |
| TP53_GOF_234 | STK11_GOF_221 | BTG1_GOF |
| NKX2-1_GOF_234 | BCOR_GOF_1526 | AR_GOF_457 |
| FH_GOF_476 | STK11_GOF_216 | MTOR_GOF_1834 |
| KEAP1_GOF_274 | | DDR2_GOF |
| NBN_GOF_219 | | KEAP1_GOF_362 |
| PAX5_GOF | | SF3B1_GOF_666 |
| TP53_GOF_195 | | KEAP1_GOF_470 |
| FANCG_GOF | | NOTCH1_GOF |
| NFE2L2_GOF_30 | | STK11_GOF_181 |
| | | KDM5C_GOF_1330 |
| | | SMAD4_GOF |
| | | RAD51B_LOF |
| | | FGF6_GOF |
| | | IDH1_GOF |
| | | MLH1_GOF |
| | | CEBPA_GOF_197 |
| | | CHEK2_GOF_367 |
| | | MAP3K1_GOF |
| cluster4 | cluster5 | cluster6 |
| NF1_LOF | NF1_LOF | NF1_LOF |
| ASXL1_LOF | STK11_LOF | ASXL1_LOF |
| TSC2_LOF | BRAF_GOF_600 | ATRX_LOF |
| BRAF_GOF_600 | ASXL1_LOF | BRAF_GOF_600 |
| STK11_LOF | TSC2_LOF | STK11_LOF |
| KMT2A_LOF | ATR_LOF | BRCA2_LOF |
| ATRX_LOF | KMT2A_LOF | TSC2_LOF |
| BRCA2_LOF | BRCA2_LOF | FGF19_LOF |
| PAX5_GOF | PDGFRA_LOF | PDK1_LOF |
| STAG2_LOF | NFKBIA_GOF | NFE2L2_GOF_24 |
| FGF19_LOF | ATRX_LOF | ATR_LOF |
| QKI_LOF | STAG2_LOF | NFKBIA_GOF |
| CDKN2A_GOF_151 | NFE2L2_GOF_24 | CDKN2A_GOF_151 |
| U2AF1_GOF_34 | NKX2-1_LOF | NKX2-1_LOF |
| PDK1_LOF | PDK1_LOF | DNMT3A_GOF_882 |
| NFE2L2_GOF_24 | FGF19_LOF | STAG2_LOF |
| DNMT3A_GOF_882 | U2AF1_GOF_34 | MYCN_LOF |
| EGFR_GOF_719 | FANCG_GOF | KMT2A_LOF |
| CDH1_LOF | CDKN2A_GOF_151 | PPP2R1A_GOF |
| TP53_GOF_331 | NRAS_LOF | U2AF1_GOF_34 |
| MYD88_GOF_265 | TP53_GOF_282 | PAX5_GOF |
| NRAS_LOF | CEBPA_LOF | JAK1_GOF |
| NKX2-1_LOF | FGF14_GOF | NFE2L2_GOF_77 |
| GATA3_GOF | EGER_GOF_719 | GATA3_GOF |
| PPP2R1A_GOF | GATA3_GOF | EGER_GOF_719 |
| PTEN_GOF | MDM2_LOF | TP53_GOF_282 |
| NFKBIA_GOF | EGFR_GOF_746 | TP53_GOF_244 |
| PIK3C2G_GOF_1088 | ACVR1B_LOF | TP53_GOF_331 |
| TP53_GOF_95 | EGFR_GOF_747 | FGF14_GOF |
| CDKN2A_GOF_80 | TNFAIP3_GOF | BRAF_GOF_466 |
| CD79A_LOF | CDK4_GOF | PBRM1_LOF |
| NBN_LOF | ZNF703_GOF | JUN_LOF |
| TP53_GOF_282 | CHEK1_LOF | MUTYH_GOF_165 |
| EGER_GOF_747 | ERCC4_LOF | MAP3K1_GOF |
| BRAF_GOF_469 | TP53_GOF_285 | AKT3_GOF |
| IDH1_GOF | MTOR_GOF_1834 | TP53_GOF_376 |
| NTRK2_LOF | PPP2R1A_GOF | PTEN_GOF |
| TP53_GOF_244 | BRAF_GOF_469 | STK11_GOF_291 |
| CD274_GOF | NFE2L2_GOF_77 | RAD51B_LOF |
| FGFR4_GOF | KDM5C_GOF_1546 | EGFR_GOF_746 |
| KEAP1_GOF_260 | MYD88_GOF_265 | TP53_GOF_215 |
| STK11_GOF_181 | MAP3K13_GOF | KDM5C_GOF_1330 |
| STK11_GOF_291 | TP53_GOF_278 | AR_GOF_457 |
| CDK4_GOF | SMARCA4_GOF_1157 | EGFR_GOF_747 |
| RB1_GOF | CCNE1_GOF | PPARG_LOF |
| TNFAIP3_GOF | RAD52_GOF | PARP1_GOF |
| ZNF703_GOF | IDH1_GOF | IDH1_GOF |
| FGF14_GOF | NOTCH1_GOF | CDKN1A_GOF |
| SOX2_LOF | AR_GOF_493 | PIK3CA_GOF_1043 |
| TP53_GOF_238 | TP53_GOF_192 | GATA6_GOF |
| BRD4_GOF | FLCN_GOF_306 | BRAF_GOF_469 |
| JAK1_GOF | BRCA2_GOF | EPHA3_GOF |
| BRCA2_GOF | MYCN_LOF | MTOR_GOF_1834 |
| BRAF_GOF_466 | RNF43_GOF | TP53_GOF_275 |
| SF3B1_GOF_666 | KEAP1_GOF_470 | STK11_GOF_37 |
| PRKAR1A_GOF | KEAP1_GOF_544 | TP53_GOF_195 |
| PTEN_GOF_59 | STK11_GOF_242 | ERCC4_LOF |
| AMER1_GOF_625 | SF3B1_GOF_666 | TP53_GOF_236 |
| PARP1_GOF | CD79A_LOF | ACVR1B_LOF |
| NFE2L2_GOF_29 | HRAS_GOF_61 | DIS3_GOF_458 |
| SDHA_GOF_531 | NTRK3_GOF | SMAD4_GOF |
| STK11_GOF_199 | PIK3CA_GOF_1043 | MAP3K1_GOF_949 |
| JAK3_GOF | RBM10_GOF | BCORL1_GOF_883 |
| ACVR1B_LOF | NF1_GOF_1642 | FH_GOF_476 |
| KDM6A_GOF | CUL4A_LOF | PIK3C2G_GOF_129 |
| KEAP1_GOF | TP53_GOF_159 | TP53_GOF_272 |
| CUL4A_LOF | MLH1_LOF | CDKN2A_GOF_100 |
| KEAP1_GOF_364 | NFE2L2_GOF_31 | GABRA6_GOF |
| SMARCA4_GOF_1243 | CTNNA1_LOF | MAP2K1_GOF_102 |
| KEAP1_GOF_186 | DNMT3A_GOF_749 | CDH1_LOF |
| EGER_GOF_771 | STK11_GOF_734 | NRAS_LOF |
| NFE2L2_GOF_77 | ARID1A_GOF_21 | FLT3_GOF |
| RBM10_GOF_503 | STK11_GOF_168 | GNAQ_GOF |
| EGFR_GOF_861 | STK11_GOF_256 | KEAP1_GOF_135 |
| TP53_GOF_560 | FH_GOF | TP53_GOF_285 |
| MED12_GOF | IRF4_GOF | ATM_GOF |
| RAD51D_GOF | ARID1A_GOF_343 | ZNF703_GOF |
| PIK3CA_GOF_726 | STK11_GOF_291 | STK11_GOF_163 |
| KMT2D_GOF_755 | MYCN_GOF_44 | SMARCA4_GOF_1157 |
| CTCF_LOF | RARA_GOF | RPTOR_GOF |
| ATM_GOF | PTCH1_GOF_48 | KEAP1_GOF_470 |
| NFE2L2_GOF_30 | DNMT3A_GOF_882 | KEAP1_GOF_274 |
| BCOR_GOF_679 | RAD54L_LOF | NOTCH1_GOF |
| STK11_GOF_37 | KEAP1_GOF_523 | KEAP1_GOF_509 |
| HRAS_LOF | MITF_GOF | |
| RAD51B_LOF | TYRO3_GOF | |
| IRF4_GOF | ARID1A_GOF_515 | |
| EGFR_GOF_746 | GABRA6_GOF | |
| MUTYH_GOF_165 | RB1_GOF_576 | |
| KDM5C_GOF | | |
| EGFR_GOF_763 | | |
| BCL6_GOF | | |
| FH_GOF_476 | | |
| cluster7 | cluster8 | cluster9 |
| NF1_LOF | NF1_LOF | NF1_LOF |
| ATR_LOF | KMT2A_LOF | KMT2A_LOF |
| STK11_LOF | STK11_LOF | BRAF_GOF_600 |
| ASXL1_LOF | BRAF_GOF_600 | STK11_LOF |
| BRAF_GOF_600 | ASXL1_LOF | ASXL1_LOF |
| ATRX_LOF | PDGFRA_LOF | TSC2_LOF |
| FGF19_LOF | ATR_LOF | FGF19_LOF |
| BRCA2_LOF | FGF19_LOF | PDGFRA_LOF |
| TSC2_LOF | BRCA2_LOF | MYCN_LOF |
| NKX2-1_LOF | TSC2_LOF | NFKBIA_GOF |
| NFKBIA_GOF | NFKBIA_GOF | PDK1_LOF |
| CDKN2A_GOF_151 | PDK1_LOF | CDKN2A_GOF_151 |
| KMT2A_LOF | NKX2-1_LOF | WT1_LOF |
| PDK1_LOF | NFE2L2_GOF_24 | TP53_GOF_282 |
| JAK1_GOF | MYCN_LOF | VEGFA_LOF |
| NFE2L2_GOF_77 | TP53_GOF_331 | NKX2-1_LOF |
| STAG2_LOF | WT1_LOF | ATR_LOF |
| BRAF_GOF_466 | STAG2_LOF | DNMT3A_GOF_882 |
| MUTYH_GOF_165 | TP53_GOF_282 | PPP2R1A_GOF |
| TP53_GOF_282 | CDKN2A_GOF_151 | STAG2_LOF |
| NFE2L2_GOF_24 | QKI_LOF | EGFR_GOF_719 |
| PTEN_GOF | PPP2R1A_GOF | NFE2L2_GOF_24 |
| MAP3K1_GOF | EGFR_GOF_719 | U2AF1_GOF_34 |
| DNMT3A_GOF_882 | U2AF1_GOF_34 | TP53_GOF_331 |
| PAX5_GOF | DNMT3A_GOF_882 | NFE2L2_GOF_77 |
| U2AF1_GOF_34 | GATA3_GOF | CDC73_GOF |
| TP53_GOF_376 | HGF_LOF | BRCA2_LOF |
| FGF14_GOF | NRAS_LOF | KEAP1_GOF_260 |
| TP53_GOF_244 | PIK3C2G_GOF_1088 | CCNE1_GOF |
| MYCN_LOF | FANCG_GOF | TP53_GOF_560 |
| TP53_GOF_215 | STK11_GOF_291 | BRAF_GOF_466 |
| TP53_GOF_331 | TP53_GOF_159 | TP53_GOF_159 |
| JUN_LOF | MTOR_GOF_1834 | FANCG_GOF |
| GATA3_GOF | VEGFA_LOF | EGFR_GOF_746 |
| STK11_GOF_291 | TP53_GOF_272 | MTOR_GOF_1834 |
| EGFR_GOF_747 | JUN_LOF | JUN_LOF |
| BRAF_GOF_469 | MYCN_GOF | HGF_LOF |
| EGFR_GOF_719 | PIK3CA_GOF_1043 | STK11_GOF_291 |
| PPP2R1A_GOF | AR_GOF_493 | PIK3C2G_GOF_1088 |
| IDH1_GOF | ERBB2_GOF_755 | KDM5C_GOF_1546 |
| AKT3_GOF | CEBPA_LOF | AR_GOF_69 |
| CDKN1A_GOF | BRAF_GOF_66 | EGER_GOF_858 |
| PPARG_LOF | SF3B1_GOF_666 | ZNF703_GOF |
| PBRM1_LOF | PTEN_GOF_59 | MYCN_GOF |
| KDM5C_GOF_1330 | KEAP1_GOF_364 | BRCA2_GOF |
| AR_GOF_457 | STK11_GOF_221 | GATA3_GOF |
| PIK3CA_GOF_1043 | ARID1A_GOF_21 | FGF6_GOF |
| GATA6_GOF | TNFRSF14_LOF | IRF4_GOF |
| EGFR_GOF_46 | MAP2K1_GOF_102 | STK11_GOF_84 |
| ACVR1B_LOF | STK11_GOF_251 | TP53_GOF_376 |
| ERCC4_LOF | TP53_GOF_278 | KEAP1_GOF_272 |
| PIK3C2G_GOF_129 | ERBB2_GOF_776 | MAP3K1_GOF |
| PARP1_GOF | NFE2L2_GOF_77 | KEAP1_GOF_332 |
| RAD51B_LOF | KEAP1_GOF | TNFAIP3_GOF |
| TP53_GOF_236 | RB1_GOF | PIK3CA_GOF_1043 |
| MTOR_GOF_834 | AKT3_GOF | KEAP1_GOF_234 |
| TP53_GOF_195 | MYD88_GOF_265 | KEAP1_GOF_364 |
| MAP3K1_GOF_949 | KEAP1_GOF_509 | SMARCB1_GOF |
| SMAD4_GOF | ATM_GOF_337 | CEBPA_LOF |
| MAP2K1_GOF_102 | SMARCA4_GOF_1160 | KLHL6_GOF |
| CDH1_LOF | CUL4A_LOF | QKI_LOF |
| NRAS_LOF | FLT1_GOF | AKT1_LOF |
| RPTOR_GOF | EGFR_GOF_746 | PPARG_GOF |
| CDKN2A_GOF_100 | PIK3R1_LOF | ATRX_GOF |
| KEAP1_GOF_409 | KEAP1_GOF_430 | FBXW7_GOF_505 |
| TP53_GOF_285 | ALOX12B_GOF | TP53_GOF_195 |
| GNAQ_GOF | KEAP1_GOF_234 | MET_GOF_3028 |
| TP53_GOF_275 | APC_GOF | STK11_GOF_163 |
| DIS3_GOF_458 | RAF1_GOF | IRF2_GOF |
| GABRA6_GOF | PARP1_GOF | AR_GOF_457 |
| ATM_GOF | TP53_GOF_195 | SMARCA4_GOF_1162 |
| STK11_GOF_37 | NFE2L2_GOF_30 | ERBB2_GOF_755 |
| BCORL1_GOF_883 | KEAP1_GOF_272 | CREBBP_GOF_1472 |
| ZNF703_GOF | CD79A_LOF | |
| EGFR_GOF_858 | MDM2_LOF | |
| EPHA3_GOF | KEAP1_GOF_493 | |
| PTEN_GOF_165 | CTNNB1_GOF_33 | |
| GNAS_GOF_415 | STK11_GOF_464 | |
| KEAP1_GOF_470 | | |
| KEAP1_GOF_274 | | |
| KEAP1_GOF_509 | | |
| NTRK2_LOF | | |
| FH_GOF_476 | | |
| CDKN2A_GOF_61 | | |
| MYCN_GOF | | |
| POLD1_GOF | | |
| FAS_GOF | | |
| SMAD2_LOF | | |
| cluster10 | cluster11 | cluster12 |
| STK11_LOF | NF1_LOF | NF1_LOF |
| NF1_LOF | STK11_LOF | ATR_LOF |
| BRAF_GOF_600 | BRAF_GOF_600 | STK11_LOF |
| ATR_LOF | ASXL1_LOF | BRAF_GOF_600 |
| BRCA2_LOF | STAG2_LOF | ASXL1_LOF |
| TSC2_LOF | TSC2_LOF | ATRX_LOF |
| ASXL1_LOF | FGF19_LOF | NFKBIA_GOF |
| CDKN2A_GOF_151 | NFE2L2_GOF_24 | FGF19_LOF |
| STAG2_LOF | CDKN2A_GOF_151 | BRCA2_LOF |
| FGF19_LOF | QKI_LOF | NKX2-1_LOF |
| NFE2L2_GOF_24 | TP53_GOF_95 | KMT2A_LOF |
| PDK1_LOF | NKX2-1_LOF | TSC2_LOF |
| NKX2-1_LOF | U2AF1_GOF_34 | BRAF_GOF_466 |
| DNMT3A_GOF_882 | NRAS_LOF | STAG2_LOF |
| TP53_GOF_282 | BRCA2_LOF | DNMT3A_GOF_882 |
| NFE2L2_GOF_77 | PDK1_LOF | MUTYH_GOF_165 |
| QKI_LOF | ATRX_LOF | CDKN2A_GOF_151 |
| U2AF1_GOF_34 | NFKBIA_GOF | NFE2L2_GOF_24 |
| VEGFA_LOF | STK11_GOF_291 | PDK1_LOF |
| MAP3K13_GOF | FANCG_GOF | MYCN_LOF |
| PIK3C2G_GOF_1088 | DNMT3A_GOF_882 | TP53_GOF_376 |
| JUN_LOF | NBN_LOF | JAK1_GOF |
| PPP2R1A_GOF | PDGFRA_LOF | NFE2L2_GOF_77 |
| KEAP1_GOF_272 | PIK3CA_GOF | PAX5_GOF |
| RAC1_LOF | GATA3_GOF | TP53_GOF_282 |
| TP53_GOF_195 | EGFR_GOF_719 | FGF14_GOF |
| PBRM1_LOF | PPP2R1A_GOF | U2AF1_GOF_34 |
| TP53_GOF_331 | MYCN_GOF | MAP3K1_GOF |
| AKT2_GOF | RB1_GOF | IDH1_GOF |
| NKX2-1_GOF_234 | JUN_LOF | PIK3CA_GOF_1043 |
| FGF14_GOF | TP53_GOF_282 | GATA3_GOF |
| EGFR_GOF_858 | TP53_GOF_331 | PTEN_GOF |
| KEAP1_GOF_364 | PIK3C2G_GOF_1088 | TP53_GOF_331 |
| STK11_GOF_37 | TP53_GOF_159 | JUN_LOF |
| JAK1_GOF | KMT2A_LOF | AKT3_GOF |
| TP53_GOF_285 | TNFAIP3_GOF | TP53_GOF_215 |
| EGER_GOF_747 | KEAP1_GOF_417 | EGFR_GOF_719 |
| MAP3K1_GOF | NFE2L2_GOF_77 | KDM5C_GOF_1330 |
| EGFR_GOF_719 | STK11_GOF_84 | PPARG_LOF |
| MTOR_GOF_1834 | PDCD1LG2_GOF | STK11_GOF_291 |
| KMT2A_LOF | KLHL6_GOF | PBRM1_LOF |
| BRAF_GOF_466 | PBRM1_LOF | BRAF_GOF_469 |
| CDKN1A_GOF | NTRK2_LOF | EGFR_GOF_746 |
| CTNNA1_LOF | TP53_GOF_298 | EGFR_GOF_747 |
| NTRK2_LOF | POLD1_GOF | RAD51B_LOF |
| ATRX_LOF | EGFR_GOF_746 | AR_GOF_457 |
| MUTYH_GOF_165 | VEGFA_LOF | PPP2R1A_GOF |
| SF3B1_GOF_666 | CDK4_GOF | ACVR1B_LOF |
| FGFR4_GOF | CD79B_GOF | GATA6_GOF |
| ERCC4_LOF | MYD88_GOF_265 | PARP1_GOF |
| TP53_GOF_159 | STK11_GOF_37 | SMAD4_GOF |
| MET_GOF_2888 | ERBB3_LOF | NRAS_LOF |
| GNAQ_GOF | KEAP1_GOF_272 | PIK3C2G_GOF_129 |
| FH_GOF | BRD4_GOF | ERCC4_LOF |
| ERBB2_GOF_776 | MAP3K1_GOF | MTOR_GOF_1834 |
| PIK3CA_GOF_1043 | TP53_GOF_272 | TP53_GOF_N44 |
| APC_GOF | GATA6_GOF | CDKN1A_GOF |
| AR_GOF_468 | JAK1_GOF | MAP2K1_GOF_102 |
| CDC73_GOF | FGF14_GOF | TP53_GOF_285 |
| NFKBIA_GOF | AKT3_GOF | GNAQ_GOF |
| XRCC2_LOF | AR_GOF_69 | MAP3K1_GOF_949 |
| STK11_GOF_220 | ACVR1B_LOF | EPHA3_GOF |
| IDH1_GOF | AR_GOF_469 | KEAP1_GOF_274 |
| KEAP1_GOF_332 | CDKN1A_GOF | ATM_GOF |
| NFE2L2_GOF_29 | BRAF_GOF_469 | RPTOR_GOF |
| FANCL_LOF | PTCH1_GOF | KEAP1_GOF_409 |
| FBXW7_GOF_505 | KEAP1_GOF_332 | PTEN_GOF_165 |
| KEAP1_GOF_234 | NFE2L2_GOF_27 | TP53_GOF_275 |
| CHEK2_GOF | RET_GOF_511 | SMAD2_LOF |
| MAP2K1_GOF_121 | DIS3_GOF_458 | CDH1_LOF |
| FAS_GOF | AR_GOF_598 | FANCL_LOF |
| RPTOR_GOF | MYD88_GOF | FGF6_GOF |
| AKT3_GOF | RAD51C_GOF_21 | FLT3_GOF |
| SMAD4_GOF_351 | BRAF_GOF_466 | TP53_GOF_236 |
| KEAP1_GOF_155 | STK11_GOF_242 | TP53_GOF_195 |
| IRS2_GOF | ERBB2_GOF_776 | CDKN2A_GOF_100 |
| KEAP1_GOF_274 | EGER_GOF_858 | MEN1_GOF |
| PDGFRA_LOF | AKT1_GOF_17 | CEBPA_LOF |
| SMAD2_GOF_464 | AKT2_GOF | POLD1_GOF |
| TNFAIP3_GOF | TP53_GOF_673 | FH_GOF_476 |
| CD79A_LOF | CBL_GOF_1096 | DIS3_GOF_458 |
| MITF_GOF | BCOR_GOF_679 | KMT2A_GOF_53 |
| TP53_GOF_342 | PALB2_GOF | CDK8_GOF |
| HRAS_GOF_13 | CDKN2A_GOF_69 | PIK3CA_GOF_545 |
| KEAP1_GOF_362 | KEAP1_GOF_450 | STK11_GOF_168 |
| BCL2_LOF | CDKN2A_GOF_61 | KEAP1_GOF |
| NTRK3_GOF_610 | BTG1_GOF | STK11_GOF_176 |
| SMARCB1_GOF | VHL_GOF | KEAP1_GOF_450 |
| FH_GOF_476 | KEAP1_GOF_116 | GNAS_GOF_415 |
| RB1_GOF_576 | | CDKN2A_GOF_83 |
| CEBPA_LOF | | MDM2_LOF |
| KLHL6_GOF | | KEAP1_GOF_236 |
| AR_GOF_467 | | STK11_GOF_163 |
| BCORL1_GOF_94 | | IDH2_GOF_140 |

TABLE 4
Overlap between binary, GoF/LoF, hotspot and overall mutations.
Feature popularity within 12 models

| Binary | GOF/LOF | Hotspot | Overlap Between |
|---|---|---|---|
| Overlap | Overlap | Overlap | 36 models |
| NF1 | NF1_LOF | NF1_LOF | NF1 |
| STK11 | STK11_LOF | STK11_LOF | STK11 |
| TSC2 | ASXL1_LOF | ASXL1_LOF | TSC2 |
| BRCA2 | FGF19_LOF | KMT2A_LOF | BRCA2 |
| BRAF | TSC2_LOF | NFKBIA_GOF | BRAF |
| STAG2 | BRAF_GOF | BRAF_GOF_600 | STAG2 |
| U2AF1 | IDH1_GOF | TSC2_LOF | U2AF1 |
| PDK1 | STAG2_LOF | FGF19_LOF | PDK1 |
| ATR | BRCA2_LOF | NKX2-1_LOF | |
| | PDK1_LOF | BRCA2_LOF | |
| | U2AF1_GOF | CDKN2A_GOF_151 | |
| | NKX2-1_LOF | PDK1_LOF | |
| | PPP2R1A_GOF | TP53_GOF_282 | |
| | | NFE2L2_GOF_24 | |
| | | U2AF1_GOF_34 | |
| | | EGFR_GOF_719 | |
| | | PPP2R1A_GOF | |
| | | DNMT3A_GOF_882 | |
| | | STAG2_LOF | |

TABLE 5
Overlap between all inputs.
Overlap Between all Inputs

NF1
STK11
TSC2
BRCA2
BRAF
ATRX
STAG2
U2AF1
PDK1
ATR
ASXL1
ERCC4
PAX5
CTNNA1
CD79A
TSC1
NRAS
RARA
PDCD1LG2
NBN
PDGFRB
PDGFRA
CCNE1
JUN
IDH1
CDK4
NKX2-1
PPP2R1A
FH
MDM2
AKT1
NTRK2
FANCG
QKI
BRD4
CDKN1A
CEBPA
FANCL
SMARCA4

TABLE 6
Coefficient information for the durable response group.
Durable Response

| | | | | Percent |
|---|---|---|---|---|
| | | Contri- | | In |
| | | bution | | popu- |
| | | To | | lation |
| Gene_Input | GeneName | Response | Input | (n = 8768) |
| TSC2 | TSC2 | 1.559808 | binary | 5.132299 |
| U2AF1 | U2AF1 | 1.348334 | binary | 2.463504 |
| FGF23 | FGF23 | 1.266593 | binary | 3.61542 |
| IDH1 | IDH1 | 1.266593 | binary | 1.687956 |
| PDCD1LG2 | PDCD1LG2 | 1.23884 | binary | 1.687956 |
| MEF2B | MEF2B | 1.215131 | binary | 1.448449 |
| PDK1 | PDK1 | 1.16345 | binary | 1.676551 |
| BRIP1 | BRIP1 | 1.152226 | binary | 4.17427 |
| QKI | QKI | 1.064153 | binary | 1.231752 |
| CTNNA1 | CTNNA1 | 1.064153 | binary | 1.859033 |
| FUBP1 | FUBP1 | 0.978551 | binary | 2.018704 |
| STAG2 | STAG2 | 0.972096 | binary | 4.493613 |
| FANCL | FANCL | 0.933488 | binary | 1.995894 |
| PAX5 | PAX5 | 0.925331 | binary | 3.193431 |
| MLH1 | MLH1 | 0.856218 | binary | 1.687956 |
| FANCG | FANCG | 0.849644 | binary | 2.212591 |
| AKT1 | AKT1 | 0.842021 | binary | 1.893248 |
| MPL | MPL | 0.842021 | binary | 2.703011 |
| BRCA2 | BRCA2 | 0.830051 | binary | 7.071168 |
| ATR | ATR | 0.803612 | binary | 8.81615 |
| POLE | POLE | 0.77999 | binary | 7.538777 |
| TSC1 | TSC1 | 0.778369 | binary | 3.033759 |
| FOXL2 | FOXL2 | 0.758752 | binary | 2.292427 |
| BRAF | BRAF | 0.717131 | binary | 7.607208 |
| ASXL1 | ASXL1 | 0.702532 | binary | 6.375456 |
| NF1 | NF1 | 0.700892 | binary | 12.44297 |
| ATRX | ATRX | 0.668535 | binary | 9.899635 |
| NRAS | NRAS | 0.659455 | binary | 1.254562 |
| PDGFRA | PDGFRA | 0.657149 | binary | 8.37135 |
| SMAD4 | SMAD4 | 0.63135 | binary | 4.64188 |
| NBN | NBN | 0.609419 | binary | 6.238595 |
| PDGFRB | PDGFRB | 0.558338 | binary | 4.539234 |
| BRCA1 | BRCA1 | 0.539297 | binary | 4.881387 |
| TP53 | TP53 | 0.520266 | binary | 66.40055 |
| SMARCA4 | SMARCA4 | 0.059468 | binary | 12.60265 |
| FGF19_LOF | FGF19 | 2.172156 | goflof | 6.021898 |
| U2AF1_GOF | U2AF1 | 1.634661 | goflof | 2.463504 |
| NBN_LOF | NBN | 1.621183 | goflof | 6.238595 |
| NRAS_LOF | NRAS | 1.456333 | goflof | 1.254562 |
| RAC1_LOF | RAC1 | 1.456333 | goflof | 3.421533 |
| RB1_GOF | RB1 | 1.456333 | goflof | 9.124088 |
| IDH1_GOF | IDH1 | 1.348334 | goflof | 1.687956 |
| XRCC2_LOF | XRCC2 | 1.254342 | goflof | 1.58531 |
| PAX5_GOF | PAX5 | 1.254342 | goflof | 3.193431 |
| PDCD1LG2_GOF | PDCD1LG2 | 1.23884 | goflof | 1.687956 |
| CD274_GOF | CD274 | 1.23884 | goflof | 1.459854 |
| RAD52_GOF | RAD52 | 1.23884 | goflof | 2.771442 |
| VEGFA_LOF | VEGFA | 1.215131 | goflof | 2.577555 |
| CBFB_LOF | CBFB | 1.215131 | goflof | 1.07208 |
| AKT1_GOF | AKT1 | 1.215131 | goflof | 1.893248 |
| FGF23_LOF | FGF23 | 1.16345 | goflof | 3.61542 |
| PDK1_LOF | PDK1 | 1.16345 | goflof | 1.676551 |
| MYD88_GOF | MYD88 | 1.158903 | goflof | 1.04927 |
| CD79B_GOF | CD79B | 1.158903 | goflof | 1.60812 |
| PRKAR1A_GOF | PRKAR1A | 1.158903 | goflof | 1.380018 |
| BRCA2_GOF | BRCA2 | 1.158903 | goflof | 7.071168 |
| SDHC_LOF | SDHC | 1.158903 | goflof | 2.58896 |
| GNA13_LOF | GNA13 | 1.116719 | goflof | 1.357208 |
| FANCG_GOF | FANCG | 1.064153 | goflof | 2.212591 |
| FANCL_LOF | FANCL | 1.064153 | goflof | 1.995894 |
| SOX2_LOF | SOX2 | 1.032211 | goflof | 9.181113 |
| EZH2_LOF | EZH2 | 1.011338 | goflof | 2.531934 |
| STAG2_LOF | STAG2 | 0.972096 | goflof | 4.493613 |
| RAD51C_LOF | RAD51C | 0.954659 | goflof | 1.437044 |
| QKI_LOF | QKI | 0.954659 | goflof | 1.231752 |
| FGF6_GOF | FGF6 | 0.954659 | goflof | 3.832117 |
| FGF23_GOF | FGF23 | 0.954659 | goflof | 3.61542 |
| MUTYH_GOF | MUTYH | 0.954659 | goflof | 3.159215 |
| SYK_LOF | SYK | 0.941318 | goflof | 1.630931 |
| CTNNA1_LOF | CTNNA1 | 0.941318 | goflof | 1.859033 |
| CDH1_LOF | CDH1 | 0.928907 | goflof | 2.12135 |
| PBRM1_LOF | PBRM1 | 0.877942 | goflof | 4.812956 |
| PIK3C2G_GOF | PIK3C2G | 0.867665 | goflof | 9.42062 |
| ASXL1_LOF | ASXL1 | 0.852463 | goflof | 6.375456 |
| BRAF_GOF | BRAF | 0.844869 | goflof | 7.607208 |
| NF1_LOF | NF1 | 0.839896 | goflof | 12.44297 |
| CHEK1_LOF | CHEK1 | 0.832538 | goflof | 1.528285 |
| FUBP1_LOF | FUBP1 | 0.832538 | goflof | 2.018704 |
| ERRFI1_LOF | ERRFI1 | 0.832538 | goflof | 1.58531 |
| BRCA2_LOF | BRCA2 | 0.822594 | goflof | 7.071168 |
| HSD3B1_GOF | HSD3B1 | 0.81931 | goflof | 3.113595 |
| DNMT3A_GOF | DNMT3A | 0.81931 | goflof | 8.827555 |
| WT1_LOF | WT1 | 0.800475 | goflof | 4.698905 |
| PDGFRA_LOF | PDGFRA | 0.793705 | goflof | 8.37135 |
| ATRX_LOF | ATRX | 0.743512 | goflof | 9.899635 |
| ATR_LOF | ATR | 0.739747 | goflof | 8.81615 |
| RBM10_GOF | RBM10 | 0.737032 | goflof | 10.54973 |
| MAP2K2_GOF | MAP2K2 | 0.737032 | goflof | 1.106296 |
| BRIP1_GOF | BRIP1 | 0.737032 | goflof | 4.17427 |
| PDCD1LG2_LOF | PDCD1LG2 | 0.737032 | goflof | 1.687956 |
| MEF2B_GOF | MEF2B | 0.737032 | goflof | 1.448449 |
| CREBBP_GOF | CREBBP | 0.737032 | goflof | 7.652829 |
| IRF2_GOF | IRF2 | 0.737032 | goflof | 1.448449 |
| BARD1_GOF | BARD1 | 0.737032 | goflof | 3.033759 |
| CTNNA1_GOF | CTNNA1 | 0.737032 | goflof | 1.859033 |
| MPL_GOF | MPL | 0.737032 | goflof | 2.703011 |
| ACVR1B_LOF | ACVR1B | 0.732527 | goflof | 2.018704 |
| PPARG_LOF | PPARG | 0.732527 | goflof | 1.676551 |
| TSC2_LOF | TSC2 | 0.729362 | goflof | 5.132299 |
| KMT2A_LOF | KMT2A | 0.69818 | goflof | 6.683394 |
| PTPN11_LOF | PTPN11 | 0.69437 | goflof | 1.813412 |
| PDGFRB_LOF | PDGFRB | 0.669592 | goflof | 4.539234 |
| AKT1_LOF | AKT1 | 0.610406 | goflof | 1.893248 |
| CHEK2_LOF | CHEK2 | 0.603633 | goflof | 4.12865 |
| NOTCH2_GOF | NOTCH2 | 0.559568 | goflof | 7.470347 |
| MUTYH_LOF | MUTYH | 0.559568 | goflof | 3.159215 |
| TSC2_GOF | TSC2 | 0.499286 | goflof | 5.132299 |
| MAP3K1_LOF | MAP3K1 | 0.448653 | goflof | 4.356752 |
| ARID1A_GOF | ARID1A | 0.42784 | goflof | 11.88412 |
| MED12_GOF | MED12 | 0.077416 | goflof | 8.131843 |
| CBFB_GOF | CBFB | 0.077416 | goflof | 1.07208 |
| PIK3R1_GOF | PIK3R1 | 0.077416 | goflof | 2.452099 |
| STAG2_GOF | STAG2 | 1.33E−14 | goflof | 4.493613 |
| MAP2K4_GOF | MAP2K4 | 1.33E−14 | goflof | 1.847628 |
| FLCN_GOF | FLCN | 1.33E−14 | goflof | 2.12135 |
| TSC1_GOF | TSC1 | 1.33E−14 | goflof | 3.033759 |
| SETD2_GOF | SETD2 | 1.33E−14 | goflof | 7.185219 |
| FGF19_LOF | FGF19 | 2.172156 | hotspot | 6.021898 |
| TP53_GOF_331 | TP53 | 1.874747 | hotspot | 66.40055 |
| NFE2L2_GOF_24 | NFE2L2 | 1.686601 | hotspot | 6.455292 |
| PIK3C2G_GOF_1088 | PIK3C2G | 1.686601 | hotspot | 9.42062 |
| U2AF1_GOF_34 | U2AF1 | 1.634661 | hotspot | 2.463504 |
| NBN_LOF | NBN | 1.621183 | hotspot | 6.238595 |
| NFE2L2_GOF_77 | NFE2L2 | 1.456333 | hotspot | 6.455292 |
| TP53_GOF_376 | TP53 | 1.456333 | hotspot | 66.40055 |
| TP53_GOF_244 | TP53 | 1.456333 | hotspot | 66.40055 |
| AKT1_GOF_17 | AKT1 | 1.456333 | hotspot | 1.893248 |
| RAC1_LOF | RAC1 | 1.456333 | hotspot | 3.421533 |
| MYCN_GOF | MYCN | 1.456333 | hotspot | 2.999544 |
| CDKN2A_GOF_151 | CDKN2A | 1.456333 | hotspot | 30.31478 |
| DNMT3A_GOF_882 | DNMT3A | 1.456333 | hotspot | 8.827555 |
| NRAS_LOF | NRAS | 1.456333 | hotspot | 1.254562 |
| XRCC2_LOF | XRCC2 | 1.254342 | hotspot | 1.58531 |
| PAX5_GOF | PAX5 | 1.254342 | hotspot | 3.193431 |
| BRAF_GOF_600 | BRAF | 1.254342 | hotspot | 7.607208 |
| RAD52_GOF | RAD52 | 1.23884 | hotspot | 2.771442 |
| PDCD1LG2_GOF | PDCD1LG2 | 1.23884 | hotspot | 1.687956 |
| CD274_GOF | CD274 | 1.23884 | hotspot | 1.459854 |
| TP53_GOF_159 | TP53 | 1.215131 | hotspot | 66.40055 |
| VEGFA_LOF | VEGFA | 1.215131 | hotspot | 2.577555 |
| CBFB_LOF | CBFB | 1.215131 | hotspot | 1.07208 |
| PDK1_LOF | PDK1 | 1.16345 | hotspot | 1.676551 |
| KDM5C_GOF_1330 | KDM5C | 1.158903 | hotspot | 5.782391 |
| KEAP1_GOF_332 | KEAP1 | 1.158903 | hotspot | 18.56752 |
| STK11_GOF_37 | STK11 | 1.158903 | hotspot | 17.49544 |
| BRCA2_GOF | BRCA2 | 1.158903 | hotspot | 7.071168 |
| KEAP1_GOF_116 | KEAP1 | 1.158903 | hotspot | 18.56752 |
| MYD88_GOF_265 | MYD88 | 1.158903 | hotspot | 1.04927 |
| IDH1_GOF | IDH1 | 1.158903 | hotspot | 1.687956 |
| AR_GOF_457 | AR | 1.158903 | hotspot | 8.177464 |
| RB1_GOF | RB1 | 1.158903 | hotspot | 9.124088 |
| MUTYH_GOF_165 | MUTYH | 1.158903 | hotspot | 3.159215 |
| BRAF_GOF_466 | BRAF | 1.158903 | hotspot | 7.607208 |
| POLE_GOF | POLE | 1.158903 | hotspot | 7.538777 |
| TP53_GOF_673 | TP53 | 1.158903 | hotspot | 66.40055 |
| CD79B_GOF | CD79B | 1.158903 | hotspot | 1.60812 |
| ARID1A_GOF_21 | ARID1A | 1.158903 | hotspot | 11.88412 |
| PRKAR1A_GOF | PRKAR1A | 1.158903 | hotspot | 1.380018 |
| DIS3_GOF_458 | DIS3 | 1.158903 | hotspot | 3.501369 |
| CDKN2A_GOF_69 | CDKN2A | 1.158903 | hotspot | 30.31478 |
| FANCL_LOF | FANCL | 1.064153 | hotspot | 1.995894 |
| FANCG_GOF | FANCG | 1.064153 | hotspot | 2.212591 |
| SOX2_LOF | SOX2 | 1.032211 | hotspot | 9.181113 |
| SMAD4_GOF | SMAD4 | 1.026984 | hotspot | 4.64188 |
| BRAF_GOF_469 | BRAF | 1.026984 | hotspot | 7.607208 |
| TP53_GOF_275 | TP53 | 1.026984 | hotspot | 66.40055 |
| TP53_GOF_192 | TP53 | 1.026984 | hotspot | 66.40055 |
| STAG2_LOF | STAG2 | 0.972096 | hotspot | 4.493613 |
| QKI_LOF | QKI | 0.954659 | hotspot | 1.231752 |
| FGF6_GOF | FGF6 | 0.954659 | hotspot | 3.832117 |
| CTNNA1_LOF | CTNNA1 | 0.941318 | hotspot | 1.859033 |
| MLH1_LOF | MLH1 | 0.928907 | hotspot | 1.687956 |
| CDH1_LOF | CDH1 | 0.928907 | hotspot | 2.12135 |
| ERBB3_LOF | ERBB3 | 0.926409 | hotspot | 3.706661 |
| PBRM1_LOF | PBRM1 | 0.877942 | hotspot | 4.812956 |
| ASXL1_LOF | ASXL1 | 0.852463 | hotspot | 6.375456 |
| KDM5A_GOF | KDM5A | 0.842021 | hotspot | 5.885037 |
| NF1_LOF | NF1 | 0.839896 | hotspot | 12.44297 |
| CHEK1_LOF | CHEK1 | 0.832538 | hotspot | 1.528285 |
| BRCA2_LOF | BRCA2 | 0.822594 | hotspot | 7.071168 |
| TP53_GOF_560 | TP53 | 0.81931 | hotspot | 66.40055 |
| WT1_LOF | WT1 | 0.800475 | hotspot | 4.698905 |
| SDHB_LOF | SDHB | 0.796717 | hotspot | 0.638686 |
| EGFR_GOF_858 | EGFR | 0.796717 | hotspot | 19.34307 |
| TP53_GOF_215 | TP53 | 0.796717 | hotspot | 66.40055 |
| PDGFRA_LOF | PDGFRA | 0.793705 | hotspot | 8.37135 |
| ATRX_LOF | ATRX | 0.743512 | hotspot | 9.899635 |
| ATR_LOF | ATR | 0.739747 | hotspot | 8.81615 |
| IRF2_GOF | IRF2 | 0.737032 | hotspot | 1.448449 |
| EGFR_GOF_771 | EGFR | 0.737032 | hotspot | 19.34307 |
| EGFR_GOF_861 | EGFR | 0.737032 | hotspot | 19.34307 |
| MLH1_GOF | MLH1 | 0.737032 | hotspot | 1.687956 |
| AR_GOF_465 | AR | 0.737032 | hotspot | 8.177464 |
| STK11_GOF_221 | STK11 | 0.737032 | hotspot | 17.49544 |
| FAS_GOF | FAS | 0.737032 | hotspot | 1.00365 |
| AMER1_GOF_385 | AMER1 | 0.737032 | hotspot | 7.960766 |
| RBM10_GOF_503 | RBM10 | 0.737032 | hotspot | 10.54973 |
| KEAP1_GOF_153 | KEAP1 | 0.737032 | hotspot | 18.56752 |
| STK11_GOF_199 | STK11 | 0.737032 | hotspot | 17.49544 |
| PTCH1_GOF | PTCH1 | 0.737032 | hotspot | 4.550639 |
| STK11_GOF_163 | STK11 | 0.737032 | hotspot | 17.49544 |
| ATRX_GOF | ATRX | 0.737032 | hotspot | 9.899635 |
| KEAP1_GOF_509 | KEAP1 | 0.737032 | hotspot | 18.56752 |
| TP53_GOF_236 | TP53 | 0.737032 | hotspot | 66.40055 |
| KEAP1_GOF_362 | KEAP1 | 0.737032 | hotspot | 18.56752 |
| HSD3B1_GOF_75 | HSD3B1 | 0.737032 | hotspot | 3.113595 |
| RB1_GOF_576 | RB1 | 0.737032 | hotspot | 9.124088 |
| STK11_GOF_216 | STK11 | 0.737032 | hotspot | 17.49544 |
| FBXW7_GOF_479 | FBXW7 | 0.737032 | hotspot | 3.934763 |
| KEAP1_GOF_523 | KEAP1 | 0.737032 | hotspot | 18.56752 |
| JAK3_GOF | JAK3 | 0.737032 | hotspot | 4.276916 |
| PPARG_LOF | PPARG | 0.732527 | hotspot | 1.676551 |
| PIK3CB_GOF | PIK3CB | 0.732527 | hotspot | 3.980383 |
| ACVR1B_LOF | ACVR1B | 0.732527 | hotspot | 2.018704 |
| TSC2_LOF | TSC2 | 0.729362 | hotspot | 5.132299 |
| KMT2A_LOF | KMT2A | 0.69818 | hotspot | 6.683394 |
| TP53_GOF_238 | TP53 | 0.631163 | hotspot | 66.40055 |
| AKT1_LOF | AKT1 | 0.610406 | hotspot | 1.893248 |
| PIK3R1_LOF | PIK3R1 | 0.567122 | hotspot | 2.452099 |
| IRS2_GOF | IRS2 | 0.499286 | hotspot | 8.200274 |
| HRAS_GOF_61 | HRAS | 0.499286 | hotspot | 0.969434 |
| PIK3CA_GOF_726 | PIK3CA | 0.499286 | hotspot | 14.18796 |
| AR_GOF_468 | AR | 0.499286 | hotspot | 8.177464 |
| TNFRSF14_LOF | TNFRSF14 | 0.400896 | hotspot | 1.094891 |
| NFE2L2_GOF_29 | NFE2L2 | 0.103465 | hotspot | 6.455292 |
| MEN1_GOF | MEN1 | 0.103465 | hotspot | 2.007299 |
| BCL2_LOF | BCL2 | 0.103465 | hotspot | 0.809763 |
| AR_GOF_467 | AR | 0.077416 | hotspot | 8.177464 |
| ATM_GOF_337 | ATM | 0.077416 | hotspot | 11.91834 |
| KEAP1_GOF_544 | KEAP1 | 0.077416 | hotspot | 18.56752 |
| NTRK3_GOF | NTRK3 | 0.077416 | hotspot | 9.876825 |
| NTRK3_GOF_610 | NTRK3 | 0.077416 | hotspot | 9.876825 |
| HRAS_LOF | HRAS | 0.077416 | hotspot | 0.969434 |
| CTNNB1_GOF_41 | CTNNB1 | 1.33E−14 | hotspot | 4.15146 |
| SMARCA4_GOF_1160 | SMARCA4 | 1.33E−14 | hotspot | 12.60265 |
| KEAP1_GOF_409 | KEAP1 | 1.33E−14 | hotspot | 18.56752 |
| SMAD2_GOF_464 | SMAD2 | 1.33E−14 | hotspot | 1.5625 |
| PTEN_GOF_59 | PTEN | 1.33E−14 | hotspot | 6.683394 |
| CTNNB1_GOF_33 | CTNNB1 | 1.33E−14 | hotspot | 4.15146 |
| CREBBP_GOF_1472 | CREBBP | 1.33E−14 | hotspot | 7.652829 |
| APC_GOF | APC | 1.33E−14 | hotspot | 7.937956 |
| PTEN_GOF_165 | PTEN | 1.33E−14 | hotspot | 6.683394 |
| TSC1_GOF | TSC1 | 1.33E−14 | hotspot | 3.033759 |
| KEAP1_GOF_417 | KEAP1 | 1.33E−14 | hotspot | 18.56752 |
| RBM10_GOF | RBM10 | 1.33E−14 | hotspot | 10.54973 |
| NBN_GOF_219 | NBN | 1.33E−14 | hotspot | 6.238595 |
| PIK3CA_GOF_345 | PIK3CA | 1.33E−14 | hotspot | 14.18796 |
| AR_GOF_493 | AR | 1.33E−14 | hotspot | 8.177464 |
| ZNF217_GOF_410 | ZNF217 | 1.33E−14 | hotspot | 4.345347 |
| AR_GOF_598 | AR | 1.33E−14 | hotspot | 8.177464 |
| STK11_GOF_256 | STK11 | 1.33E−14 | hotspot | 17.49544 |
| ARID1A_GOF_343 | ARID1A | 1.33E−14 | hotspot | 11.88412 |
| RET_GOF_511 | RET | 1.33E−14 | hotspot | 6.592153 |
| KEAP1_GOF_155 | KEAP1 | 1.33E−14 | hotspot | 18.56752 |
| MITF_GOF | MITF | 1.33E−14 | hotspot | 1.836223 |
| CDK8_GOF | CDK8 | 1.33E−14 | hotspot | 1.186131 |
| DNMT3A_GOF_749 | DNMT3A | 1.33E−14 | hotspot | 8.827555 |
| MYD88_GOF | MYD88 | 1.33E−14 | hotspot | 1.04927 |
| KEAP1_GOF_430 | KEAP1 | 1.33E−14 | hotspot | 18.56752 |
| GNAS_GOF_415 | GNAS | 1.33E−14 | hotspot | 10.28741 |
| KDM5C_GOF | KDM5C | 1.33E−14 | hotspot | 5.782391 |
| FLT1_GOF | FLT1 | 1.33E−14 | hotspot | 7.036953 |
| NF1_GOF_1642 | NF1 | 1.33E−14 | hotspot | 12.44297 |
| KMT2A_GOF_53 | KMT2A | 1.33E−14 | hotspot | 6.683394 |
| SDHA_GOF_531 | SDHA | 1.33E−14 | hotspot | 12.10082 |
| CDKN2A_GOF_61 | CDKN2A | 1.33E−14 | hotspot | 30.31478 |
| CEBPA_GOF_197 | CEBPA | 1.33E−14 | hotspot | 3.854927 |
| STK11_GOF_220 | STK11 | 1.33E−14 | hotspot | 17.49544 |
| NBN_GOF_680 | NBN | 1.33E−14 | hotspot | 6.238595 |
| KEAP1_GOF_450 | KEAP1 | 1.33E−14 | hotspot | 18.56752 |
| CHEK2_GOF_392 | CHEK2 | 1.33E−14 | hotspot | 4.12865 |
| NKX2-1_GOF_234 | NKX2-1 | 1.33E−14 | hotspot | 10.20757 |
| SMARCA4_GOF_1157 | SMARCA4 | 1.33E−14 | hotspot | 12.60265 |
| BCORL1_GOF_94 | BCORL1 | 1.33E−14 | hotspot | 8.32573 |
| KEAP1_GOF_493 | KEAP1 | 1.33E−14 | hotspot | 18.56752 |
| FLCN_GOF_306 | FLCN | 1.33E−14 | hotspot | 2.12135 |
| STK11_GOF_168 | STK11 | 1.33E−14 | hotspot | 17.49544 |
| MSH6_GOF_1088 | MSH6 | 1.33E−14 | hotspot | 3.410128 |

TABLE 7
Coefficient information for the innate CPI resistance group.
Innate Resistance

| | | Contri- | | Percent In |
|---|---|---|---|---|
| | | bution | | popu- |
| | Gene | To | | lation |
| Gene_Input | Name | Response | Input | (n = 8768) |
| GID4 | GID4 | −1.56175 | binary | 1.060675 |
| JUN | JUN | −0.82472 | binary | 1.345803 |
| RAD51 | RAD51 | −0.82472 | binary | 0.752737 |
| CD79A | CD79A | −0.69758 | binary | 1.414234 |
| STK11 | STK11 | −0.66955 | binary | 17.49544 |
| TET2 | TET2 | −0.58201 | binary | 6.626369 |
| MAP2K1 | MAP2K1 | −0.57806 | binary | 1.893248 |
| CCNE1 | CCNE1 | −0.574 | binary | 4.208485 |
| CDK4 | CDK4 | −0.57279 | binary | 3.820712 |
| ERCC4 | ERCC4 | −0.57279 | binary | 2.988139 |
| CEBPA | CEBPA | −0.52738 | binary | 3.854927 |
| RARA | RARA | −0.50609 | binary | 2.383668 |
| CDKN1A | CDKN1A | −0.4431 | binary | 1.02646 |
| RAD51B | RAD51B | −0.4431 | binary | 1.334398 |
| PPP2R1A | PPP2R1A | −0.38647 | binary | 2.349453 |
| CSF1R | CSF1R | −0.38397 | binary | 3.832117 |
| FH | FH | −0.36521 | binary | 3.63823 |
| NKX2-1 | NKX2-1 | −0.358 | binary | 10.20757 |
| NTRK2 | NTRK2 | −0.34154 | binary | 3.558394 |
| FGFR1 | FGFR1 | −0.23631 | binary | 6.25 |
| CDK6 | CDK6 | −0.15361 | binary | 3.467153 |
| MDM2 | MDM2 | −0.15062 | binary | 5.964872 |
| BRD4 | BRD4 | −0.06751 | binary | 3.980383 |
| MAP3K1_GOF | MAP3K1 | −1.74432 | goflof | 4.356752 |
| FGF14_GOF | FGF14 | −1.56175 | goflof | 2.976734 |
| PPP2R1A_GOF | PPP2R1A | −1.56175 | goflof | 2.349453 |
| CDKN1A_GOF | CDKN1A | −1.33962 | goflof | 1.02646 |
| JAK1_GOF | JAK1 | −1.33962 | goflof | 2.714416 |
| IRF4_GOF | IRF4 | −1.33962 | goflof | 2.782847 |
| JUN_LOF | JUN | −1.1624 | goflof | 1.345803 |
| AKT3_GOF | AKT3 | −1.1624 | goflof | 3.489964 |
| NKX2-1_LOF | NKX2-1 | −1.15045 | goflof | 10.20757 |
| TGFBR2_GOF | TGFBR2 | −1.05544 | goflof | 2.953923 |
| GABRA6_GOF | GABRA6 | −1.05544 | goflof | 4.881387 |
| BCORL1_GOF | BCORL1 | −1.05544 | goflof | 8.32573 |
| TNFAIP3_GOF | TNFAIP3 | −1.05544 | goflof | 2.395073 |
| POLD1_GOF | POLD1 | −1.05544 | goflof | 4.71031 |
| GNAQ_GOF | GNAQ | −1.05544 | goflof | 0.775547 |
| RAD51C_GOF | RAD51C | −1.05544 | goflof | 1.437044 |
| BRD4_GOF | BRD4 | −1.05544 | goflof | 3.980383 |
| RNF43_GOF | RNF43 | −1.05544 | goflof | 2.657391 |
| FGFR4_GOF | FGFR4 | −1.05544 | goflof | 3.387318 |
| GATA6_GOF | GATA6 | −1.00728 | goflof | 3.832117 |
| GATA3_GOF | GATA3 | −1.00728 | goflof | 4.037409 |
| MDM2_LOF | MDM2 | −1.00728 | goflof | 5.964872 |
| MYC_LOF | MYC | −0.99501 | goflof | 8.565237 |
| RPTOR_GOF | RPTOR | −0.82472 | goflof | 4.19708 |
| CD79A_LOF | CD79A | −0.82472 | goflof | 1.414234 |
| CCNE1_GOF | CCNE1 | −0.73203 | goflof | 4.208485 |
| STK11_LOF | STK11 | −0.72345 | goflof | 17.49544 |
| MYCN_LOF | MYCN | −0.71132 | goflof | 2.999544 |
| CEBPA_LOF | CEBPA | −0.69758 | goflof | 3.854927 |
| PARP1_GOF | PARP1 | −0.69758 | goflof | 3.330292 |
| NOTCH3_GOF | NOTCH3 | −0.65962 | goflof | 8.30292 |
| SDHD_GOF | SDHD | −0.65962 | goflof | 0.55885 |
| LTK_GOF | LTK | −0.65962 | goflof | 2.63458 |
| DAXX_GOF | DAXX | −0.65962 | goflof | 2.61177 |
| ABL1_GOF | ABL1 | −0.65962 | goflof | 3.284672 |
| PDGFRB_GOF | PDGFRB | −0.65962 | goflof | 4.539234 |
| BTG1_GOF | BTG1 | −0.65962 | goflof | 0.775547 |
| CHEK1_GOF | CHEK1 | −0.65962 | goflof | 1.528285 |
| GATA4_GOF | GATA4 | −0.65962 | goflof | 2.862683 |
| JUN_GOF | JUN | −0.65962 | goflof | 1.345803 |
| FLT3_GOF | FLT3 | −0.65962 | goflof | 4.562044 |
| CUL3_GOF | CUL3 | −0.65962 | goflof | 3.067975 |
| CDK4_GOF | CDK4 | −0.65552 | goflof | 3.820712 |
| AKT2_GOF | AKT2 | −0.63635 | goflof | 3.546989 |
| KDM6A_GOF | KDM6A | −0.60259 | goflof | 4.812956 |
| MTOR_GOF | MTOR | −0.60259 | goflof | 6.090329 |
| BCL2L1_LOF | BCL2L1 | −0.60259 | goflof | 2.09854 |
| CD274_LOF | CD274 | −0.60259 | goflof | 1.459854 |
| NF2_GOF | NF2 | −0.58541 | goflof | 2.908303 |
| SMARCA4_GOF | SMARCA4 | −0.57806 | goflof | 12.60265 |
| NFKBIA_GOF | NFKBIA | −0.57663 | goflof | 6.979927 |
| ERCC4_LOF | ERCC4 | −0.57314 | goflof | 2.988139 |
| ZNF703_GOF | ZNF703 | −0.52122 | goflof | 5.554288 |
| STK11_GOF | STK11 | −0.51839 | goflof | 17.49544 |
| KEAP1_GOF | KEAP1 | −0.50849 | goflof | 18.56752 |
| ERBB2_GOF | ERBB2 | −0.49443 | goflof | 6.421077 |
| FH_GOF | FH | −0.4839 | goflof | 3.63823 |
| REL_LOF | REL | −0.4431 | goflof | 1.859033 |
| RARA_GOF | RARA | −0.4431 | goflof | 2.383668 |
| RAD51_LOF | RAD51 | −0.40285 | goflof | 0.752737 |
| PTEN_GOF | PTEN | −0.40285 | goflof | 6.683394 |
| NTRK2_LOF | NTRK2 | −0.4019 | goflof | 3.558394 |
| CDKN2B_LOF | CDKN2B | −0.37499 | goflof | 17.4042 |
| BAP1_LOF | BAP1 | −0.36521 | goflof | 2.372263 |
| RARA_LOF | RARA | −0.35811 | goflof | 2.383668 |
| AXL_GOF | AXL | −0.34779 | goflof | 3.341697 |
| CCND2_LOF | CCND2 | −0.34779 | goflof | 2.828467 |
| CUL4A_LOF | CUL4A | −0.34779 | goflof | 2.543339 |
| H3F3A_GOF | H3F3A | −0.34779 | goflof | 1.197537 |
| GRM3_GOF | GRM3 | −0.32465 | goflof | 8.759124 |
| CDK6_GOF | CDK6 | −0.32465 | goflof | 3.467153 |
| ARFRP1_LOF | ARFRP1 | −0.31841 | goflof | 2.497719 |
| SUFU_LOF | SUFU | −0.28798 | goflof | 1.391423 |
| RAD54L_LOF | RAD54L | −0.27916 | goflof | 1.881843 |
| PIK3CB_LOF | PIK3CB | −0.27448 | goflof | 3.980383 |
| KLHL6_GOF | KLHL6 | −0.2703 | goflof | 9.021442 |
| MAP3K13_GOF | MAP3K13 | −0.2318 | goflof | 7.527372 |
| PIK3CA_GOF | PIK3CA | −0.22932 | goflof | 14.18796 |
| CDKN2A_LOF | CDKN2A | −0.22309 | goflof | 30.31478 |
| EZH2_GOF | EZH2 | −0.21284 | goflof | 2.531934 |
| FANCA_LOF | FANCA | −0.20211 | goflof | 4.482208 |
| HGF_LOF | HGF | −0.1972 | goflof | 9.990876 |
| FBXW7_GOF | FBXW7 | −0.18072 | goflof | 3.934763 |
| FGFR2_GOF | FGFR2 | −0.18072 | goflof | 2.782847 |
| AURKA_LOF | AURKA | −0.18072 | goflof | 1.744982 |
| HSD3B1_LOF | HSD3B1 | −0.15965 | goflof | 3.113595 |
| SDHC_GOF | SDHC | −0.10371 | goflof | 2.58896 |
| EGFR_GOF_746 | EGFR | −1.89944 | hotspot | 19.34307 |
| TP53_GOF_282 | TP53 | −1.74432 | hotspot | 66.40055 |
| EGFR_GOF_747 | EGFR | −1.56175 | hotspot | 19.34307 |
| EGFR_GOF_719 | EGFR | −1.56175 | hotspot | 19.34307 |
| TP53_GOF_195 | TP53 | −1.56175 | hotspot | 66.40055 |
| FGF14_GOF | FGF14 | −1.56175 | hotspot | 2.976734 |
| PPP2R1A_GOF | PPP2R1A | −1.56175 | hotspot | 2.349453 |
| IRF4_GOF | IRF4 | −1.33962 | hotspot | 2.782847 |
| MAP3K1_GOF | MAP3K1 | −1.33962 | hotspot | 4.356752 |
| STK11_GOF_291 | STK11 | −1.33962 | hotspot | 17.49544 |
| ERBB2_GOF_776 | ERBB2 | −1.33962 | hotspot | 6.421077 |
| JAK1_GOF | JAK1 | −1.33962 | hotspot | 2.714416 |
| CDKN1A_GOF | CDKN1A | −1.33962 | hotspot | 1.02646 |
| KEAP1_GOF_272 | KEAP1 | −1.33962 | hotspot | 18.56752 |
| JUN_LOF | JUN | −1.1624 | hotspot | 1.345803 |
| AKT3_GOF | AKT3 | −1.1624 | hotspot | 3.489964 |
| NKX2-1_LOF | NKX2-1 | −1.15045 | hotspot | 10.20757 |
| ATM_GOF | ATM | −1.05544 | hotspot | 11.91834 |
| CDKN2A_GOF_100 | CDKN2A | −1.05544 | hotspot | 30.31478 |
| STK11_GOF_84 | STK11 | −1.05544 | hotspot | 17.49544 |
| FGFR4_GOF | FGFR4 | −1.05544 | hotspot | 3.387318 |
| KEAP1_GOF_483 | KEAP1 | −1.05544 | hotspot | 18.56752 |
| KEAP1_GOF_234 | KEAP1 | −1.05544 | hotspot | 18.56752 |
| GABRA6_GOF | GABRA6 | −1.05544 | hotspot | 4.881387 |
| CDKN2A_GOF_80 | CDKN2A | −1.05544 | hotspot | 30.31478 |
| MYCN_GOF_44 | MYCN | −1.05544 | hotspot | 2.999544 |
| KEAP1_GOF_260 | KEAP1 | −1.05544 | hotspot | 18.56752 |
| MAP2K1_GOF_102 | MAP2K1 | −1.05544 | hotspot | 1.893248 |
| PTEN_GOF | PTEN | −1.05544 | hotspot | 6.683394 |
| PTCH1_GOF_48 | PTCH1 | −1.05544 | hotspot | 4.550639 |
| GNAQ_GOF | GNAQ | −1.05544 | hotspot | 0.775547 |
| KEAP1_GOF_364 | KEAP1 | −1.05544 | hotspot | 18.56752 |
| POLD1_GOF | POLD1 | −1.05544 | hotspot | 4.71031 |
| PIK3CA_GOF_1043 | PIK3CA | −1.05544 | hotspot | 14.18796 |
| BCOR_GOF_679 | BCOR | −1.05544 | hotspot | 8.462591 |
| FH_GOF_476 | FH | −1.05544 | hotspot | 3.63823 |
| STK11_GOF_181 | STK11 | −1.05544 | hotspot | 17.49544 |
| TP53_GOF_285 | TP53 | −1.05544 | hotspot | 66.40055 |
| TNFAIP3_GOF | TNFAIP3 | −1.05544 | hotspot | 2.395073 |
| BRD4_GOF | BRD4 | −1.05544 | hotspot | 3.980383 |
| KDM5C_GOF_1546 | KDM5C | −1.05544 | hotspot | 5.782391 |
| KEAP1_GOF_274 | KEAP1 | −1.05544 | hotspot | 18.56752 |
| SMAD4_GOF_351 | SMAD4 | −1.05544 | hotspot | 4.64188 |
| RNF43_GOF | RNF43 | −1.05544 | hotspot | 2.657391 |
| SF3B1_GOF_666 | SF3B1 | −1.05544 | hotspot | 4.607664 |
| MTOR_GOF_1834 | MTOR | −1.05544 | hotspot | 6.090329 |
| NOTCH1_GOF | NOTCH1 | −1.05544 | hotspot | 9.272354 |
| TP53_GOF_272 | TP53 | −1.00728 | hotspot | 66.40055 |
| GATA6_GOF | GATA6 | −1.00728 | hotspot | 3.832117 |
| MDM2_LOF | MDM2 | −1.00728 | hotspot | 5.964872 |
| GATA3_GOF | GATA3 | −1.00728 | hotspot | 4.037409 |
| PIK3CA_GOF_1047 | PIK3CA | −0.82472 | hotspot | 14.18796 |
| TP53_GOF_278 | TP53 | −0.82472 | hotspot | 66.40055 |
| CD79A_LOF | CD79A | −0.82472 | hotspot | 1.414234 |
| RPTOR_GOF | RPTOR | −0.82472 | hotspot | 4.19708 |
| ZNF703_GOF | ZNF703 | −0.8035 | hotspot | 5.554288 |
| CCNE1_GOF | CCNE1 | −0.73203 | hotspot | 4.208485 |
| STK11_LOF | STK11 | −0.72345 | hotspot | 17.49544 |
| MYCN_LOF | MYCN | −0.71132 | hotspot | 2.999544 |
| PARP1_GOF | PARP1 | −0.69758 | hotspot | 3.330292 |
| CEBPA_LOF | CEBPA | −0.69758 | hotspot | 3.854927 |
| MAP2K1_GOF_121 | MAP2K1 | −0.65962 | hotspot | 1.893248 |
| SMARCA4_GOF_1243 | SMARCA4 | −0.65962 | hotspot | 12.60265 |
| MED12_GOF | MED12 | −0.65962 | hotspot | 8.131843 |
| KEAP1_GOF_135 | KEAP1 | −0.65962 | hotspot | 18.56752 |
| AR_GOF_69 | AR | −0.65962 | hotspot | 8.177464 |
| BTG1_GOF | BTG1 | −0.65962 | hotspot | 0.775547 |
| MAP3K1_GOF_5 | MAP3K1 | −0.65962 | hotspot | 4.356752 |
| TYRO3_GOF | TYRO3 | −0.65962 | hotspot | 3.740876 |
| ERBB2_GOF_755 | ERBB2 | −0.65962 | hotspot | 6.421077 |
| CBL_GOF_1096 | CBL | −0.65962 | hotspot | 3.649635 |
| STK11_GOF_176 | STK11 | −0.65962 | hotspot | 17.49544 |
| EGFR_GOF_790 | EGFR | −0.65962 | hotspot | 19.34307 |
| RAF1_GOF | RAF1 | −0.65962 | hotspot | 1.494069 |
| KEAP1_GOF_244 | KEAP1 | −0.65962 | hotspot | 18.56752 |
| STK11_GOF_464 | STK11 | −0.65962 | hotspot | 17.49544 |
| STK11_GOF_308 | STK11 | −0.65962 | hotspot | 17.49544 |
| KDM6A_GOF | KDM6A | −0.65962 | hotspot | 4.812956 |
| PDGFRB_GOF | PDGFRB | −0.65962 | hotspot | 4.539234 |
| BRAF_GOF_464 | BRAF | −0.65962 | hotspot | 7.607208 |
| PIK3C2G_GOF_129 | PIK3C2G | −0.65962 | hotspot | 9.42062 |
| FBXW7_GOF_505 | FBXW7 | −0.65962 | hotspot | 3.934763 |
| KEAP1_GOF_470 | KEAP1 | −0.65962 | hotspot | 18.56752 |
| ALOX12B_GOF | ALOX12B | −0.65962 | hotspot | 1.505475 |
| FLT3_GOF | FLT3 | −0.65962 | hotspot | 4.562044 |
| AMER1_GOF_625 | AMER1 | −0.65962 | hotspot | 7.960766 |
| IDH2_GOF_140 | IDH2 | −0.65962 | hotspot | 1.311588 |
| GNAS_GOF_407 | GNAS | −0.65962 | hotspot | 10.28741 |
| KEAP1_GOF_186 | KEAP1 | −0.65962 | hotspot | 18.56752 |
| BCOR_GOF_1526 | BCOR | −0.65962 | hotspot | 8.462591 |
| NFE2L2_GOF_27 | NFE2L2 | −0.65962 | hotspot | 6.455292 |
| STK11_GOF_251 | STK11 | −0.65962 | hotspot | 17.49544 |
| CHEK1_GOF | CHEK1 | −0.65962 | hotspot | 1.528285 |
| HRAS_GOF_13 | HRAS | −0.65962 | hotspot | 0.969434 |
| KEAP1_GOF_236 | KEAP1 | −0.65962 | hotspot | 18.56752 |
| GATA4_GOF | GATA4 | −0.65962 | hotspot | 2.862683 |
| MET_GOF_2888 | MET | −0.65962 | hotspot | 8.257299 |
| KMT2D_GOF_755 | KMT2D | −0.65962 | hotspot | 17.66651 |
| RAD51C_GOF_21 | RAD51C | −0.65962 | hotspot | 1.437044 |
| SMARCA4_GOF_1162 | SMARCA4 | −0.65962 | hotspot | 12.60265 |
| STK11_GOF_734 | STK11 | −0.65962 | hotspot | 17.49544 |
| MAP3K1_GOF_949 | MAP3K1 | −0.65962 | hotspot | 4.356752 |
| PALB2_GOF | PALB2 | −0.65962 | hotspot | 3.216241 |
| BCORL1_GOF_883 | BCORL1 | −0.65962 | hotspot | 8.32573 |
| KEAP1_GOF | KEAP1 | −0.65962 | hotspot | 18.56752 |
| ARID1A_GOF_515 | ARID1A | −0.65962 | hotspot | 11.88412 |
| AR_GOF_469 | AR | −0.65962 | hotspot | 8.177464 |
| EGFR_GOF_763 | EGFR | −0.65962 | hotspot | 19.34307 |
| AR_GOF_70 | AR | −0.65962 | hotspot | 8.177464 |
| PPARG_GOF | PPARG | −0.65962 | hotspot | 1.676551 |
| VHL_GOF | VHL | −0.65962 | hotspot | 1.00365 |
| PARP3_GOF | PARP3 | −0.65962 | hotspot | 1.106296 |
| CDK4_GOF | CDK4 | −0.65552 | hotspot | 3.820712 |
| AKT2_GOF | AKT2 | −0.63635 | hotspot | 3.546989 |
| TP53_GOF_920 | TP53 | −0.60259 | hotspot | 66.40055 |
| NFE2L2_GOF_30 | NFE2L2 | −0.60259 | hotspot | 6.455292 |
| CHEK2_GOF | CHEK2 | −0.60259 | hotspot | 4.12865 |
| SMAD2_LOF | SMAD2 | −0.58541 | hotspot | 1.5625 |
| SMARCB1_GOF | SMARCB1 | −0.58541 | hotspot | 1.630931 |
| TP53_GOF_298 | TP53 | −0.58541 | hotspot | 66.40055 |
| SDHA_GOF_457 | SDHA | −0.58541 | hotspot | 12.10082 |
| FH_GOF | FH | −0.57806 | hotspot | 3.63823 |
| CDC73_GOF | CDC73 | −0.57806 | hotspot | 3.136405 |
| NFKBIA_GOF | NFKBIA | −0.57663 | hotspot | 6.979927 |
| ERCC4_LOF | ERCC4 | −0.57314 | hotspot | 2.988139 |
| RARA_GOF | RARA | −0.4431 | hotspot | 2.383668 |
| RAD51B_LOF | RAD51B | −0.4431 | hotspot | 1.334398 |
| NTRK2_LOF | NTRK2 | −0.4019 | hotspot | 3.558394 |
| CUL4A_LOF | CUL4A | −0.34779 | hotspot | 2.543339 |
| STK11_GOF_57 | STK11 | −0.31841 | hotspot | 17.49544 |
| TP53_GOF_234 | TP53 | −0.31841 | hotspot | 66.40055 |
| STK11_GOF_242 | STK11 | −0.31841 | hotspot | 17.49544 |
| CHEK2_GOF_367 | CHEK2 | −0.31841 | hotspot | 4.12865 |
| RAD51D_GOF | RAD51D | −0.31841 | hotspot | 1.471259 |
| CTCF_LOF | CTCF | −0.28798 | hotspot | 2.144161 |
| EPHA3_GOF | EPHA3 | −0.28798 | hotspot | 11.04015 |
| RAD54L_LOF | RAD54L | −0.27916 | hotspot | 1.881843 |
| PIK3CB_LOF | PIK3CB | −0.27448 | hotspot | 3.980383 |
| KLHL6_GOF | KLHL6 | −0.2703 | hotspot | 9.021442 |
| PIK3CA_GOF | PIK3CA | −0.26748 | hotspot | 14.18796 |
| DDR2_GOF | DDR2 | −0.23599 | hotspot | 6.443887 |
| MAP3K13_GOF | MAP3K13 | −0.2318 | hotspot | 7.527372 |
| PIK3CA_GOF_545 | PIK3CA | −0.20301 | hotspot | 14.18796 |
| HGF_LOF | HGF | −0.1972 | hotspot | 9.990876 |
| NFE2L2_GOF_31 | NFE2L2 | −0.18072 | hotspot | 6.455292 |
| MET_GOF_3028 | MET | −0.18072 | hotspot | 8.257299 |
| BCL6_GOF | BCL6 | −0.16433 | hotspot | 7.162409 |
| TP53_GOF_342 | TP53 | −0.10542 | hotspot | 66.40055 |
| CDKN2A_GOF_83 | CDKN2A | −0.05772 | hotspot | 30.31478 |

TABLE 8
feature set scores for binary gene input.

| | | | |
|---|---|---|---|
| | Train | | |
| Train Validation | Validation | | Chemo Test |
| Score | Score | Test Score | Score |

Binary Gene Inputs Feature Set 1

| | | | |
|---|---|---|---|
| Accuracy | 0.695 ± 0.008 | 0.575 ± 0.002 | 0.471 ± 0.002 |
| F1 | 0.704 ± 0.011 | 0.599 ± 0.003 | 0.536 ± 0.002 |
| Precision | 0.677 ± 0.025 | 0.566 ± 0.002 | 0.475 ± 0.001 |
| Recall | 0.742 ± 0.012 | 0.650 ± 0.005 | 0.613 ± 0.003 |
| Matthews | 0.394 ± 0.028 | 0.152 ± 0.005 | −0.068 ± 0.004 |
| CorrCoef | | | |
| Area under ROC | 0.704 ± 0.011 | 0.596 ± 0.002 | 0.457 ± 0.002 |
| Curve | | | |

Binary Gene Inputs Feature Set 2

| | | | |
|---|---|---|---|
| Accuracy | 0.697 ± 0.014 | 0.573 ± 0.002 | 0.461 ± 0.001 |
| F1 | 0.709 ± 0.014 | 0.612 ± 0.002 | 0.543 ± 0.002 |
| Precision | 0.686 ± 0.016 | 0.553 ± 0.002 | 0.469 ± 0.001 |
| Recall | 0.742 ± 0.024 | 0.686 ± 0.005 | 0.641 ± 0.004 |
| Matthews | 0.418 ± 0.058 | 0.137 ± 0.005 | −0.084 ± 0.004 |
| CorrCoef | | | |
| Area under ROC | 0.720 ± 0.006 | 0.588 ± 0.002 | 0.451 ± 0.002 |
| Curve | | | |

Binary Gene Inputs Feature Set 3

| | | | |
|---|---|---|---|
| Accuracy | 0.703 ± 0.014 | 0.604 ± 0.002 | 0.498 ± 0.002 |
| F1 | 0.719 ± 0.017 | 0.636 ± 0.002 | 0.570 ± 0.001 |
| Precision | 0.661 ± 0.005 | 0.595 ± 0.002 | 0.502 ± 0.001 |
| Recall | 0.770 ± 0.027 | 0.677 ± 0.005 | 0.665 ± 0.003 |
| Matthews | 0.410 ± 0.020 | 0.216 ± 0.004 | −0.005 ± 0.003 |
| CorrCoef | | | |
| Area under ROC | 0.720 ± 0.015 | 0.629 ± 0.002 | 0.487 ± 0.002 |
| Curve | | | |

Binary Gene Inputs Feature Set 4

| | | | |
|---|---|---|---|
| Accuracy | 0.688 ± 0.025 | 0.595 ± 0.002 | 0.484 ± 0.002 |
| F1 | 0.716 ± 0.016 | 0.624 ± 0.002 | 0.567 ± 0.002 |
| Precision | 0.685 ± 0.022 | 0.577 ± 0.002 | 0.490 ± 0.001 |
| Recall | 0.753 ± 0.020 | 0.673 ± 0.004 | 0.670 ± 0.004 |
| Matthews | 0.405 ± 0.030 | 0.184 ± 0.005 | −0.031 ± 0.004 |
| CorrCoef | | | |
| Area under ROC | 0.718 ± 0.017 | 0.601 ± 0.002 | 0.471 ± 0.002 |
| Curve | | | |

Binary Gene Inputs Feature Set 5

| | | | |
|---|---|---|---|
| Accuracy | 0.701 ± 0.008 | 0.592 ± 0.002 | 0.507 ± 0.001 |
| F1 | 0.710 ± 0.009 | 0.626 ± 0.002 | 0.583 ± 0.002 |
| Precision | 0.682 ± 0.006 | 0.577 ± 0.002 | 0.503 ± 0.001 |
| Recall | 0.756 ± 0.027 | 0.677 ± 0.004 | 0.687 ± 0.004 |
| Matthews | 0.369 ± 0.033 | 0.188 ± 0.005 | 0.008 ± 0.003 |
| CorrCoef | | | |
| Area under ROC | 0.709 ± 0.009 | 0.617 ± 0.002 | 0.505 ± 0.001 |
| Curve | | | |

Binary Gene Inputs Feature Set 6

| | | | |
|---|---|---|---|
| Accuracy | 0.700 ± 0.013 | 0.615 ± 0.002 | 0.491 ± 0.002 |
| F1 | 0.705 ± 0.019 | 0.647 ± 0.002 | 0.567 ± 0.002 |
| Precision | 0.668 ± 0.019 | 0.605 ± 0.002 | 0.497 ± 0.001 |
| Recall | 0.759 ± 0.022 | 0.694 ± 0.004 | 0.662 ± 0.004 |
| Matthews | 0.397 ± 0.039 | 0.240 ± 0.004 | −0.013 ± 0.003 |
| CorrCoef | | | |
| Area under ROC | 0.721 ± 0.019 | 0.644 ± 0.002 | 0.492 ± 0.002 |
| Curve | | | |

Binary Gene Inputs Feature Set 7

| | | | |
|---|---|---|---|
| Accuracy | 0.700 ± 0.014 | 0.599 ± 0.002 | 0.493 ± 0.002 |
| F1 | 0.707 ± 0.013 | 0.627 ± 0.002 | 0.567 ± 0.002 |
| Precision | 0.678 ± 0.013 | 0.593 ± 0.002 | 0.495 ± 0.002 |
| Recall | 0.751 ± 0.037 | 0.677 ± 0.004 | 0.666 ± 0.004 |
| Matthews | 0.367 ± 0.018 | 0.202 ± 0.004 | −0.020 ± 0.004 |
| CorrCoef | | | |
| Area under ROC | 0.717 ± 0.006 | 0.609 ± 0.002 | 0.469 ± 0.002 |
| Curve | | | |

Binary Gene Inputs Feature Set 8

| | | | |
|---|---|---|---|
| Accuracy | 0.692 ± 0.006 | 0.594 ± 0.002 | 0.465 ± 0.001 |
| F1 | 0.707 ± 0.022 | 0.621 ± 0.002 | 0.540 ± 0.002 |
| Precision | 0.680 ± 0.022 | 0.584 ± 0.002 | 0.475 ± 0.001 |
| Recall | 0.737 ± 0.016 | 0.667 ± 0.004 | 0.632 ± 0.004 |
| Matthews | 0.402 ± 0.033 | 0.193 ± 0.005 | −0.076 ± 0.003 |
| CorrCoef | | | |
| Area under ROC | 0.713 ± 0.019 | 0.623 ± 0.002 | 0.464 ± 0.002 |
| Curve | | | |

Binary Gene Inputs Feature Set 9

| | | | |
|---|---|---|---|
| Accuracy | 0.690 ± 0.015 | 0.596 ± 0.002 | 0.461 ± 0.001 |
| F1 | 0.720 ± 0.010 | 0.635 ± 0.002 | 0.542 ± 0.002 |
| Precision | 0.671 ± 0.009 | 0.577 ± 0.002 | 0.472 ± 0.001 |
| Recall | 0.748 ± 0.027 | 0.709 ± 0.004 | 0.636 ± 0.004 |
| Matthews | 0.372 ± 0.019 | 0.206 ± 0.004 | −0.089 ± 0.003 |
| CorrCoef | | | |
| Area under ROC | 0.722 ± 0.008 | 0.621 ± 0.002 | 0.449 ± 0.001 |
| Curve | | | |

Binary Gene Inputs Feature Set 10

| | | | |
|---|---|---|---|
| Accuracy | 0.681 ± 0.015 | 0.564 ± 0.002 | 0.477 ± 0.002 |
| F1 | 0.713 ± 0.023 | 0.613 ± 0.003 | 0.553 ± 0.002 |
| Precision | 0.656 ± 0.013 | 0.552 ± 0.002 | 0.480 ± 0.001 |
| Recall | 0.759 ± 0.029 | 0.690 ± 0.005 | 0.645 ± 0.004 |
| Matthews | 0.409 ± 0.032 | 0.139 ± 0.005 | −0.059 ± 0.003 |
| CorrCoef | | | |
| Area under ROC | 0.699 ± 0.012 | 0.577 ± 0.002 | 0.479 ± 0.002 |
| Curve | | | |

Binary Gene Inputs Feature Set 11

| | | | |
|---|---|---|---|
| Accuracy | 0.707 ± 0.009 | 0.584 ± 0.003 | 0.472 ± 0.002 |
| F1 | 0.721 ± 0.014 | 0.635 ± 0.002 | 0.554 ± 0.002 |
| Precision | 0.700 ± 0.015 | 0.570 ± 0.002 | 0.478 ± 0.001 |
| Recall | 0.740 ± 0.026 | 0.724 ± 0.004 | 0.660 ± 0.003 |
| Matthews | 0.408 ± 0.042 | 0.183 ± 0.005 | −0.064 ± 0.003 |
| CorrCoef | | | |
| Area under ROC | 0.724 ± 0.010 | 0.610 ± 0.002 | 0.476 ± 0.002 |
| Curve | | | |

Binary Gene Inputs Feature Set 12

| | | | |
|---|---|---|---|
| Accuracy | 0.686 ± 0.019 | 0.579 ± 0.002 | 0.467 ± 0.002 |
| F1 | 0.707 ± 0.020 | 0.617 ± 0.002 | 0.554 ± 0.002 |
| Precision | 0.665 ± 0.016 | 0.567 ± 0.002 | 0.478 ± 0.001 |
| Recall | 0.773 ± 0.020 | 0.683 ± 0.005 | 0.667 ± 0.004 |
| Matthews | 0.392 ± 0.032 | 0.162 ± 0.004 | −0.074 ± 0.004 |
| CorrCoef | | | |
| Area under ROC | 0.717 ± 0.020 | 0.595 ± 0.002 | 0.468 ± 0.002 |
| Curve | | | |

TABLE 9
Feature scores for GoF/LoF gene input.

| | | |
|---|---|---|
| Train | | |
| Validation | | |
| Score | Test Score | Chemo Test Score |

Gain or Loss of Function Feature Set 1

| | | | |
|---|---|---|---|
| Accuracy | 0.722 ± 0.009 | 0.583 ± 0.002 | 0.498 ± 0.002 |
| F1 | 0.746 ± 0.009 | 0.598 ± 0.002 | 0.553 ± 0.002 |
| Precision | 0.714 ± 0.015 | 0.582 ± 0.002 | 0.498 ± 0.001 |
| Recall | 0.797 ± 0.010 | 0.609 ± 0.004 | 0.626 ± 0.003 |
| Matthews | 0.485 ± 0.031 | 0.171 ± 0.004 | −0.005 ± 0.003 |
| CorrCoef | | | |
| Area under ROC | 0.744 ± 0.021 | 0.612 ± 0.002 | 0.501 ± 0.002 |
| Curve | | | |

Gain or Loss of Function Feature Set 2

| | | | |
|---|---|---|---|
| Accuracy | 0.736 ± 0.020 | 0.571 ± 0.003 | 0.505 ± 0.002 |
| F1 | 0.738 ± 0.013 | 0.599 ± 0.002 | 0.565 ± 0.002 |
| Precision | 0.719 ± 0.007 | 0.562 ± 0.002 | 0.504 ± 0.001 |
| Recall | 0.778 ± 0.020 | 0.630 ± 0.004 | 0.651 ± 0.003 |
| Matthews | 0.474 ± 0.018 | 0.142 ± 0.004 | 0.009 ± 0.004 |
| CorrCoef | | | |
| Area under ROC | 0.752 ± 0.016 | 0.580 ± 0.002 | 0.514 ± 0.002 |
| Curve | | | |

Gain or Loss of Function Feature Set 3

| | | | |
|---|---|---|---|
| Accuracy | 0.732 ± 0.014 | 0.565 ± 0.002 | 0.507 ± 0.002 |
| F1 | 0.737 ± 0.011 | 0.588 ± 0.002 | 0.571 ± 0.002 |
| Precision | 0.697 ± 0.008 | 0.556 ± 0.002 | 0.508 ± 0.001 |
| Recall | 0.786 ± 0.016 | 0.642 ± 0.005 | 0.649 ± 0.003 |
| Matthews | 0.443 ± 0.037 | 0.134 ± 0.004 | 0.022 ± 0.004 |
| CorrCoef | | | |
| Area under ROC | 0.754 ± 0.015 | 0.603 ± 0.002 | 0.491 ± 0.002 |
| Curve | | | |

Gain or Loss of Function Feature Set 4

| | | | |
|---|---|---|---|
| Accuracy | 0.730 ± 0.013 | 0.557 ± 0.002 | 0.501 ± 0.002 |
| F1 | 0.732 ± 0.014 | 0.582 ± 0.003 | 0.560 ± 0.002 |
| Precision | 0.698 ± 0.011 | 0.549 ± 0.002 | 0.499 ± 0.001 |
| Recall | 0.797 ± 0.015 | 0.617 ± 0.004 | 0.635 ± 0.003 |
| Matthews | 0.444 ± 0.025 | 0.113 ± 0.004 | −0.004 ± 0.004 |
| CorrCoef | | | |
| Area under ROC | 0.749 ± 0.013 | 0.606 ± 0.002 | 0.508 ± 0.002 |
| Curve | | | |

Gain or Loss of Function Feature Set 5

| | | | |
|---|---|---|---|
| Accuracy | 0.724 ± 0.023 | 0.560 ± 0.002 | 0.508 ± 0.002 |
| F1 | 0.735 ± 0.011 | 0.572 ± 0.002 | 0.570 ± 0.002 |
| Precision | 0.716 ± 0.015 | 0.552 ± 0.002 | 0.505 ± 0.001 |
| Recall | 0.773 ± 0.013 | 0.596 ± 0.005 | 0.645 ± 0.004 |
| Matthews | 0.450 ± 0.041 | 0.115 ± 0.004 | 0.019 ± 0.003 |
| CorrCoef | | | |
| Area under ROC | 0.740 ± 0.013 | 0.613 ± 0.002 | 0.498 ± 0.002 |
| Curve | | | |

Gain or Loss of Function Feature Set 6

| | | | |
|---|---|---|---|
| Accuracy | 0.721 ± 0.017 | 0.583 ± 0.002 | 0.509 ± 0.002 |
| F1 | 0.740 ± 0.009 | 0.606 ± 0.002 | 0.583 ± 0.002 |
| Precision | 0.691 ± 0.014 | 0.578 ± 0.002 | 0.506 ± 0.001 |
| Recall | 0.770 ± 0.016 | 0.640 ± 0.004 | 0.682 ± 0.003 |
| Matthews | 0.482 ± 0.035 | 0.170 ± 0.003 | 0.020 ± 0.004 |
| CorrCoef | | | |
| Area under ROC | 0.752 ± 0.030 | 0.602 ± 0.002 | 0.503 ± 0.002 |
| Curve | | | |

Gain or Loss of Function Feature Set 7

| | | | |
|---|---|---|---|
| Accuracy | 0.728 ± 0.012 | 0.558 ± 0.002 | 0.528 ± 0.002 |
| F1 | 0.738 ± 0.015 | 0.588 ± 0.002 | 0.578 ± 0.002 |
| Precision | 0.704 ± 0.021 | 0.547 ± 0.002 | 0.523 ± 0.001 |
| Recall | 0.778 ± 0.033 | 0.621 ± 0.005 | 0.639 ± 0.004 |
| Matthews | 0.471 ± 0.041 | 0.117 ± 0.004 | 0.060 ± 0.003 |
| CorrCoef | | | |
| Area under ROC | 0.733 ± 0.017 | 0.587 ± 0.002 | 0.515 ± 0.002 |
| Curve | | | |

Gain or Loss of Function Feature Set 8

| | | | |
|---|---|---|---|
| Accuracy | 0.733 ± 0.015 | 0.598 ± 0.002 | 0.498 ± 0.001 |
| F1 | 0.737 ± 0.011 | 0.610 ± 0.003 | 0.558 ± 0.002 |
| Precision | 0.711 ± 0.012 | 0.589 ± 0.002 | 0.498 ± 0.001 |
| Recall | 0.762 ± 0.011 | 0.639 ± 0.004 | 0.635 ± 0.004 |
| Matthews | 0.446 ± 0.031 | 0.200 ± 0.005 | 0.003 ± 0.003 |
| CorrCoef | | | |
| Area under ROC | 0.760 ± 0.015 | 0.622 ± 0.002 | 0.483 ± 0.002 |
| Curve | | | |

Gain or Loss of Function Feature Set 9

| | | | |
|---|---|---|---|
| Accuracy | 0.723 ± 0.006 | 0.560 ± 0.002 | 0.514 ± 0.002 |
| F1 | 0.752 ± 0.020 | 0.585 ± 0.002 | 0.572 ± 0.002 |
| Precision | 0.727 ± 0.017 | 0.551 ± 0.002 | 0.512 ± 0.001 |
| Recall | 0.748 ± 0.033 | 0.617 ± 0.004 | 0.651 ± 0.004 |
| Matthews | 0.463 ± 0.019 | 0.117 ± 0.005 | 0.030 ± 0.004 |
| CorrCoef | | | |
| Area under ROC | 0.739 ± 0.031 | 0.572 ± 0.003 | 0.522 ± 0.002 |
| Curve | | | |

Gain or Loss of Function Feature Set 10

| | | | |
|---|---|---|---|
| Accuracy | 0.722 ± 0.008 | 0.550 ± 0.002 | 0.477 ± 0.002 |
| F1 | 0.736 ± 0.011 | 0.568 ± 0.002 | 0.548 ± 0.002 |
| Precision | 0.718 ± 0.020 | 0.544 ± 0.002 | 0.483 ± 0.001 |
| Recall | 0.778 ± 0.020 | 0.593 ± 0.003 | 0.637 ± 0.003 |
| Matthews | 0.432 ± 0.045 | 0.102 ± 0.004 | −0.043 ± 0.003 |
| CorrCoef | | | |
| Area under ROC | 0.751 ± 0.009 | 0.554 ± 0.002 | 0.468 ± 0.002 |
| Curve | | | |

Gain or Loss of Function Feature Set 11

| | | | |
|---|---|---|---|
| Accuracy | 0.697 ± 0.016 | 0.582 ± 0.002 | 0.501 ± 0.001 |
| F1 | 0.737 ± 0.006 | 0.598 ± 0.002 | 0.553 ± 0.002 |
| Precision | 0.694 ± 0.019 | 0.578 ± 0.002 | 0.501 ± 0.001 |
| Recall | 0.792 ± 0.016 | 0.615 ± 0.004 | 0.619 ± 0.003 |
| Matthews | 0.462 ± 0.021 | 0.169 ± 0.004 | 0.005 ± 0.003 |
| CorrCoef | | | |
| Area under ROC | 0.748 ± 0.007 | 0.607 ± 0.002 | 0.488 ± 0.001 |
| Curve | | | |

Gain or Loss of Function Feature Set 12

| | | | |
|---|---|---|---|
| Accuracy | 0.726 ± 0.010 | 0.576 ± 0.002 | 0.490 ± 0.001 |
| F1 | 0.746 ± 0.010 | 0.584 ± 0.003 | 0.549 ± 0.001 |
| Precision | 0.707 ± 0.028 | 0.567 ± 0.002 | 0.493 ± 0.001 |
| Recall | 0.792 ± 0.006 | 0.615 ± 0.005 | 0.617 ± 0.003 |
| Matthews | 0.453 ± 0.023 | 0.160 ± 0.004 | −0.012 ± 0.003 |
| CorrCoef | | | |
| Area under ROC | 0.750 ± 0.017 | 0.588 ± 0.002 | 0.481 ± 0.001 |
| Curve | | | |

TABLE 10
Feature set scores for hotspot gene input.

| | | |
|---|---|---|
| Train Validation | | |
| Score | Test Score | Chemo Test Score |

Gain or Loss of Function Feature Set 1

| | | | |
|---|---|---|---|
| Accuracy | 0.722 ± 0.009 | 0.583 ± 0.002 | 0.498 ± 0.002 |
| F1 | 0.746 ± 0.009 | 0.598 ± 0.002 | 0.553 ± 0.002 |
| Precision | 0.714 ± 0.015 | 0.582 ± 0.002 | 0.498 ± 0.001 |
| Recall | 0.797 ± 0.010 | 0.609 ± 0.004 | 0.626 ± 0.003 |
| Matthews | 0.485 ± 0.031 | 0.171 ± 0.004 | −0.005 ± 0.003 |
| CorrCoef | | | |
| Area under | 0.744 ± 0.021 | 0.612 ± 0.002 | 0.501 ± 0.002 |
| ROC Curve | | | |

Gain or Loss of Function Feature Set 2

| | | | |
|---|---|---|---|
| Accuracy | 0.736 ± 0.020 | 0.571 ± 0.003 | 0.505 ± 0.002 |
| F1 | 0.738 ± 0.013 | 0.599 ± 0.002 | 0.565 ± 0.002 |
| Precision | 0.719 ± 0.007 | 0.562 ± 0.002 | 0.504 ± 0.001 |
| Recall | 0.778 ± 0.020 | 0.630 ± 0.004 | 0.651 ± 0.003 |
| Matthews | 0.474 ± 0.018 | 0.142 ± 0.004 | 0.009 ± 0.004 |
| CorrCoef | | | |
| Area under | 0.752 ± 0.016 | 0.580 ± 0.002 | 0.514 ± 0.002 |
| ROC Curve | | | |

Gain or Loss of Function Feature Set 3

| | | | |
|---|---|---|---|
| Accuracy | 0.732 ± 0.014 | 0.565 ± 0.002 | 0.507 ± 0.002 |
| F1 | 0.737 ± 0.011 | 0.588 ± 0.002 | 0.571 ± 0.002 |
| Precision | 0.697 ± 0.008 | 0.556 ± 0.002 | 0.508 ± 0.001 |
| Recall | 0.786 ± 0.016 | 0.642 ± 0.005 | 0.649 ± 0.003 |
| Matthews | 0.443 ± 0.037 | 0.134 ± 0.004 | 0.022 ± 0.004 |
| CorrCoef | | | |
| Area under | 0.754 ± 0.015 | 0.603 ± 0.002 | 0.491 ± 0.002 |
| ROC Curve | | | |

Gain or Loss of Function Feature Set 4

| | | | |
|---|---|---|---|
| Accuracy | 0.730 ± 0.013 | 0.557 ± 0.002 | 0.501 ± 0.002 |
| F1 | 0.732 ± 0.014 | 0.582 ± 0.003 | 0.560 ± 0.002 |
| Precision | 0.698 ± 0.011 | 0.549 ± 0.002 | 0.499 ± 0.001 |
| Recall | 0.797 ± 0.015 | 0.617 ± 0.004 | 0.635 ± 0.003 |
| Matthews | 0.444 ± 0.025 | 0.113 ± 0.004 | −0.004 ± 0.004 |
| CorrCoef | | | |
| Area under | 0.749 ± 0.013 | 0.606 ± 0.002 | 0.508 ± 0.002 |
| ROC Curve | | | |

Gain or Loss of Function Feature Set 5

| | | | |
|---|---|---|---|
| Accuracy | 0.724 ± 0.023 | 0.560 ± 0.002 | 0.508 ± 0.002 |
| F1 | 0.735 ± 0.011 | 0.572 ± 0.002 | 0.570 ± 0.002 |
| Precision | 0.716 ± 0.015 | 0.552 ± 0.002 | 0.505 ± 0.001 |
| Recall | 0.773 ± 0.013 | 0.596 ± 0.005 | 0.645 ± 0.004 |
| Matthews | 0.450 ± 0.041 | 0.115 ± 0.004 | 0.019 ± 0.003 |
| CorrCoef | | | |
| Area under | 0.740 ± 0.013 | 0.613 ± 0.002 | 0.498 ± 0.002 |
| ROC Curve | | | |

Gain or Loss of Function Feature Set 6

| | | | |
|---|---|---|---|
| Accuracy | 0.721 ± 0.017 | 0.583 ± 0.002 | 0.509 ± 0.002 |
| F1 | 0.740 ± 0.009 | 0.606 ± 0.002 | 0.583 ± 0.002 |
| Precision | 0.691 ± 0.014 | 0.578 ± 0.002 | 0.506 ± 0.001 |
| Recall | 0.770 ± 0.016 | 0.640 ± 0.004 | 0.682 ± 0.003 |
| Matthews | 0.482 ± 0.035 | 0.170 ± 0.003 | 0.020 ± 0.004 |
| CorrCoef | | | |
| Area under | 0.752 ± 0.030 | 0.602 ± 0.002 | 0.503 ± 0.002 |
| ROC Curve | | | |

Gain or Loss of Function Feature Set 7

| | | | |
|---|---|---|---|
| Accuracy | 0.728 ± 0.012 | 0.558 ± 0.002 | 0.528 ± 0.002 |
| F1 | 0.738 ± 0.015 | 0.588 ± 0.002 | 0.578 ± 0.002 |
| Precision | 0.704 ± 0.021 | 0.547 ± 0.002 | 0.523 ± 0.001 |
| Recall | 0.778 ± 0.033 | 0.621 ± 0.005 | 0.639 ± 0.004 |
| Matthews | 0.471 ± 0.041 | 0.117 ± 0.004 | 0.060 ± 0.003 |
| CorrCoef | | | |
| Area under | 0.733 ± 0.017 | 0.587 ± 0.002 | 0.515 ± 0.002 |
| ROC Curve | | | |

Gain or Loss of Function Feature Set 8

| | | | |
|---|---|---|---|
| Accuracy | 0.733 ± 0.015 | 0.598 ± 0.002 | 0.498 ± 0.001 |
| F1 | 0.737 ± 0.011 | 0.610 ± 0.003 | 0.558 ± 0.002 |
| Precision | 0.711 ± 0.012 | 0.589 ± 0.002 | 0.498 ± 0.001 |
| Recall | 0.762 ± 0.011 | 0.639 ± 0.004 | 0.635 ± 0.004 |
| Matthews | 0.446 ± 0.031 | 0.200 ± 0.005 | 0.003 ± 0.003 |
| CorrCoef | | | |
| Area under | 0.760 ± 0.015 | 0.622 ± 0.002 | 0.483 ± 0.002 |
| ROC Curve | | | |

Gain or Loss of Function Feature Set 9

| | | | |
|---|---|---|---|
| Accuracy | 0.723 ± 0.006 | 0.560 ± 0.002 | 0.514 ± 0.002 |
| F1 | 0.752 ± 0.020 | 0.585 ± 0.002 | 0.572 ± 0.002 |
| Precision | 0.727 ± 0.017 | 0.551 ± 0.002 | 0.512 ± 0.001 |
| Recall | 0.748 ± 0.033 | 0.617 ± 0.004 | 0.651 ± 0.004 |
| Matthews | 0.463 ± 0.019 | 0.117 ± 0.005 | 0.030 ± 0.004 |
| CorrCoef | | | |
| Area under | 0.739 ± 0.031 | 0.572 ± 0.003 | 0.522 ± 0.002 |
| ROC Curve | | | |

Gain or Loss of Function Feature Set 10

| | | | |
|---|---|---|---|
| Accuracy | 0.722 ± 0.008 | 0.550 ± 0.002 | 0.477 ± 0.002 |
| F1 | 0.736 ± 0.011 | 0.568 ± 0.002 | 0.548 ± 0.002 |
| Precision | 0.718 ± 0.020 | 0.544 ± 0.002 | 0.483 ± 0.001 |
| Recall | 0.778 ± 0.020 | 0.593 ± 0.003 | 0.637 ± 0.003 |
| Matthews | 0.432 ± 0.045 | 0.102 ± 0.004 | −0.043 ± 0.003 |
| CorrCoef | | | |
| Area under | 0.751 ± 0.009 | 0.554 ± 0.002 | 0.468 ± 0.002 |
| ROC Curve | | | |

Gain or Loss of Function Feature Set 11

| | | | |
|---|---|---|---|
| Accuracy | 0.697 ± 0.016 | 0.582 ± 0.002 | 0.501 ± 0.001 |
| F1 | 0.737 ± 0.006 | 0.598 ± 0.002 | 0.553 ± 0.002 |
| Precision | 0.694 ± 0.019 | 0.578 ± 0.002 | 0.501 ± 0.001 |
| Recall | 0.792 ± 0.016 | 0.615 ± 0.004 | 0.619 ± 0.003 |
| Matthews | 0.462 ± 0.021 | 0.169 ± 0.004 | 0.005 ± 0.003 |
| CorrCoef | | | |
| Area under | 0.748 ± 0.007 | 0.607 ± 0.002 | 0.488 ± 0.001 |
| ROC Curve | | | |

Gain or Loss of Function Feature Set 12

| | | | |
|---|---|---|---|
| Accuracy | 0.726 ± 0.010 | 0.576 ± 0.002 | 0.490 ± 0.001 |
| F1 | 0.746 ± 0.010 | 0.584 ± 0.003 | 0.549 ± 0.001 |
| Precision | 0.707 ± 0.028 | 0.567 ± 0.002 | 0.493 ± 0.001 |
| Recall | 0.792 ± 0.006 | 0.615 ± 0.005 | 0.617 ± 0.003 |
| Matthews | 0.453 ± 0.023 | 0.160 ± 0.004 | −0.012 ± 0.003 |
| CorrCoef | | | |
| Area under | 0.750 ± 0.017 | 0.588 ± 0.002 | 0.481 ± 0.001 |
| ROC Curve | | | |

TABLE 11
Gene ID information

| Gene_name | Transcript ID |
|---|---|
| ABL1 | NM_005157 |
| ABL1 | NM_007313 |
| ACVR1B | NM_020328 |
| AKT1 | NM_001014431 |
| AKT2 | NM_001626 |
| AKT3 | NM_005465 |
| AKT3 | NM_181690 |
| ALK | NM_004304 |
| ALOX12B | NM_001139 |
| APC | NM_000038 |
| AR | NM_000044 |
| AR | NM_001011645 |
| ARAF | NM_001654 |
| ARFRP1 | NM_003224 |
| ARFRP1 | NM_001134758 |
| ARID1A | NM_006015 |
| ARID1A | NM_139135 |
| ASXL1 | NM_015338 |
| ASXL1 | NM_001164603 |
| ATM | NM_000051 |
| ATR | NM_001184 |
| ATRX | NM_000489 |
| ATRX | NM_138270 |
| AURKA | NM_003600 |
| AURKB | NM_004217 |
| AXIN1 | NM_003502 |
| AXL | NM_001699 |
| AXL | NM_021913 |
| BAP1 | NM_004656 |
| BARD1 | NM_000465 |
| BCL2 | NM_000633 |
| BCL2 | NM_000657 |
| BCL2L1 | NM_138578 |
| BCL2L2 | NM_004050 |
| BCL6 | NM_001706 |
| BCOR | NM_017745 |
| BCOR | NM_001123385 |
| BCORL1 | NM_021946 |
| BRAF | NM_004333 |
| BRCA1 | NM_007294 |
| BRCA1 | NM_007300 |
| BRCA1 | NM_007299 |
| BRCA2 | NM_000059 |
| BRD4 | NM_014299 |
| BRD4 | NM_058243 |
| BRIP1 | NM_032043 |
| BTG1 | NM_001731 |
| BTK | NM_000061 |
| CARD11 | NM_032415 |
| CASP8 | NM_001228 |
| CASP8 | NM_033358 |
| CASP8 | NM_001080125 |
| CBFB | NM_022845 |
| CBFB | NM_001755 |
| CBL | NM_005188 |
| CCND1 | NM_053056 |
| CCND2 | NM_001759 |
| CCND3 | NM_001760 |
| CCND3 | NM_001136017 |
| CCNE1 | NM_001238 |
| CD274 | NM_014143 |
| CD79A | NM_001783 |
| CD79B | NM_000626 |
| CDC73 | NM_024529 |
| CDH1 | NM_004360 |
| CDK12 | NM_016507 |
| CDK4 | NM_000075 |
| CDK6 | NM_001259 |
| CDK8 | NM_001260 |
| CDKN1A | NM_000389 |
| CDKN1B | NM_004064 |
| CDKN2A | NM_000077 |
| CDKN2A | NM_058195 |
| CDKN2A | NM_058197 |
| CDKN2B | NM_078487 |
| CDKN2B | NM_004936 |
| CDKN2C | NM_001262 |
| CEBPA | NM_004364 |
| CHEK1 | NM_001274 |
| CHEK2 | NM_007194 |
| CHEK2 | NM_001005735 |
| CIC | NM_015125 |
| CIC | NM_001304815 |
| CREBBP | NM_004380 |
| CRKL | NM_005207 |
| CSF1R | NM_005211 |
| CTCF | NM_006565 |
| CTCF | NM_001191022 |
| CTNNA1 | NM_001903 |
| CTNNB1 | NM_001904 |
| CUL3 | NM_003590 |
| CUL4A | NM_003589 |
| CUL4A | NM_001008895 |
| CYP17A1 | NM_000102 |
| DAXX | NM_001350 |
| DAXX | NM_001141970 |
| DDR1 | NM_001954 |
| DDR1 | NM_001202523 |
| DDR1 | NM_013994 |
| DDR1 | NM_001202522 |
| DDR2 | NM_006182 |
| DIS3 | NM_001128226 |
| DIS3 | NM_014953 |
| DNMT3A | NM_022552 |
| DNMT3A | NM_175630 |
| DOT1L | NM_032482 |
| EGFR | NM_005228 |
| EGFR | NM_201284 |
| EGFR | NM_201283 |
| EMSY | NM_020193 |
| EP300 | NM_001429 |
| EPHA3 | NM_005233 |
| EPHA3 | NM_182644 |
| EPHB1 | NM_004441 |
| EPHB4 | NM_004444 |
| ERBB2 | NM_004448 |
| ERBB3 | NM_001982 |
| ERBB3 | NM_001005915 |
| ERBB4 | NM_005235 |
| ERCC4 | NM_005236 |
| ERG | NM_182918 |
| ERG | NM_001136154 |
| ERRFI1 | NM_018948 |
| ESR1 | NM_000125 |
| EZH2 | NM_004456 |
| EZH2 | NM_001203249 |
| FANCA | NM_000135 |
| FANCC | NM_000136 |
| FANCG | NM_004629 |
| FANCL | NM_018062 |
| FANCL | NM_001114636 |
| FAS | NM_000043 |
| FAS | NM_152872 |
| FBXW7 | NM_033632 |
| FBXW7 | NM_018315 |
| FBXW7 | NM_001013415 |
| FGF10 | NM_004465 |
| FGF12 | NM_021032 |
| FGF12 | NM_004113 |
| FGF14 | NM_004115 |
| FGF14 | NM_175929 |
| FGF19 | NM_005117 |
| FGF23 | NM_020638 |
| FGF3 | NM_005247 |
| FGF4 | NM_002007 |
| FGF6 | NM_020996 |
| FGFR1 | NM_023110 |
| FGFR1 | NM_001174067 |
| FGFR1 | NM_001174065 |
| FGFR2 | NM_022970 |
| FGFR2 | NM_000141 |
| FGFR2 | NM_001144919 |
| FGFR3 | NM_000142 |
| FGFR4 | NM_022963 |
| FGFR4 | NM_002011 |
| FGFR4 | NM_213647 |
| FH | NM_000143 |
| FLCN | NM_144997 |
| FLCN | NM_144606 |
| FLT1 | NM_002019 |
| FLT1 | NM_001159920 |
| FLT1 | NM_001160030 |
| FLT3 | NM_004119 |
| FOXL2 | NM_023067 |
| FUBP1 | NM_003902 |
| GABRA6 | NM_000811 |
| GATA3 | NM_001002295 |
| GATA3 | NM_002051 |
| GATA4 | NM_002052 |
| GATA6 | NM_005257 |
| GNA11 | NM_002067 |
| GNA13 | NM_006572 |
| GNAQ | NM_002072 |
| GNAS | NM_000516 |
| GNAS | NM_080425 |
| GNAS | NM_016592 |
| GNAS | NM_001077490 |
| GRM3 | NM_000840 |
| GSK3B | NM_002093 |
| H3F3A | NM_002107 |
| HGF | NM_000601 |
| HGF | NM_001010931 |
| HNF1A | NM_000545 |
| HRAS | NM_176795 |
| HRAS | NM_005343 |
| HSD3B1 | NM_000862 |
| IDH1 | NM_005896 |
| IDH2 | NM_002168 |
| IGF1R | NM_000875 |
| IKBKE | NM_014002 |
| IKZF1 | NM_006060 |
| INPP4B | NM_003866 |
| IRF2 | NM_002199 |
| IRF4 | NM_002460 |
| IRS2 | NM_003749 |
| JAK1 | NM_002227 |
| JAK2 | NM_004972 |
| JAK3 | NM_000215 |
| JUN | NM_002228 |
| KDM5A | NM_001042603 |
| KDM5C | NM_004187 |
| KDM5C | NM_001146702 |
| KDM6A | NM_021140 |
| KDR | NM_002253 |
| KEAP1 | NM_012289 |
| KEL | NM_000420 |
| KIT | NM_000222 |
| KLHL6 | NM_130446 |
| KMT2D | NM_003482 |
| KRAS | NM_004985 |
| KRAS | NM_033360 |
| LTK | NM_002344 |
| LTK | NM_001135685 |
| LYN | NM_002350 |
| LYN | NM_001111097 |
| MAP2K1 | NM_002755 |
| MAP2K2 | NM_030662 |
| MAP2K4 | NM_003010 |
| MAP3K1 | NM_005921 |
| MAP3K13 | NM_004721 |
| MCL1 | NM_182763 |
| MCL1 | NM_001197320 |
| MDM2 | NM_002392 |
| MDM4 | NM_002393 |
| MED12 | NM_005120 |
| MEF2B | NM_001145785 |
| MEN1 | NM_130801 |
| MERTK | NM_006343 |
| MET | NM_000245 |
| MET | NM_001127500 |
| MITF | NM_198159 |
| MITF | NM_006722 |
| MITF | NM_000248 |
| MITF | NM_198177 |
| MKNK1 | NM_003684 |
| MKNK1 | NM_198973 |
| MLH1 | NM_000249 |
| MPL | NM_005373 |
| MSH2 | NM_000251 |
| MSH6 | NM_000179 |
| MST1R | NM_002447 |
| MTOR | NM_004958 |
| MUTYH | NM_001048171 |
| MUTYH | NM_001128425 |
| MUTYH | NM_001048172 |
| MYC | NM_002467 |
| MYCN | NM_005378 |
| MYD88 | NM_002468 |
| MYD88 | NM_001172568 |
| MYD88 | NM_001172567 |
| NBN | NM_002485 |
| NF1 | NM_001042492 |
| NF1 | NM_001128147 |
| NF2 | NM_000268 |
| NF2 | NM_181830 |
| NFE2L2 | NM_006164 |
| NFE2L2 | NM_001145412 |
| NFKBIA | NM_020529 |
| NKX2-1 | NM_003317 |
| NKX2-1 | NM_001079668 |
| NOTCH1 | NM_017617 |
| NOTCH2 | NM_024408 |
| NOTCH2 | NM_001200001 |
| NOTCH3 | NM_000435 |
| NPM1 | NM_002520 |
| NPM1 | NM_001037738 |
| NRAS | NM_002524 |
| NTRK1 | NM_002529 |
| NTRK1 | NM_001007792 |
| NTRK2 | NM_006180 |
| NTRK3 | NM_001007156 |
| NTRK3 | NM_001012338 |
| NTRK3 | NM_002530 |
| PALB2 | NM_024675 |
| PARP1 | NM_001618 |
| PARP2 | NM_005484 |
| PARP3 | NM_005485 |
| PARP3 | NM_001003931 |
| PAX5 | NM_016734 |
| PBRM1 | NM_018313 |
| PBRM1 | NM_181042 |
| PDCD1LG2 | NM_025239 |
| PDGFRA | NM_006206 |
| PDGFRB | NM_002609 |
| PDK1 | NM_002610 |
| PIK3C2B | NM_002646 |
| PIK3C2G | NM_004570 |
| PIK3CA | NM_006218 |
| PIK3CB | NM_006219 |
| PIK3R1 | NM_181523 |
| PIK3R1 | NM_181504 |
| PIK3R1 | NM_181524 |
| PMS2 | NM_000535 |
| POLD1 | NM_002691 |
| POLE | NM_006231 |
| PPARG | NM_015869 |
| PPP2R1A | NM_014225 |
| PRDM1 | NM_001198 |
| PRKAR1A | NM_212472 |
| PRKCI | NM_002740 |
| PTCH1 | NM_000264 |
| PTCH1 | NM_001083603 |
| PTEN | NM_000314 |
| PTPN11 | NM_002834 |
| QKI | NM_206854 |
| QKI | NM_006775 |
| QKI | NM_206853 |
| QKI | NM_206855 |
| RAC1 | NM_006908 |
| RAC1 | NM_018890 |
| RAD51 | NM_133487 |
| RAD51 | NM_001164270 |
| RAD51 | NM_002875 |
| RAD51B | NM_133509 |
| RAD51C | NM_058216 |
| RAD51D | NM_002878 |
| RAD51D | NM_001142571 |
| RAD52 | NM_134424 |
| RAD54L | NM_003579 |
| RAF1 | NM_002880 |
| RARA | NM_000964 |
| RARA | NM_001024809 |
| RB1 | NM_000321 |
| RBM10 | NM_005676 |
| REL | NM_002908 |
| RET | NM_020975 |
| RET | NM_020630 |
| RICTOR | NM_152756 |
| RNF43 | NM_017763 |
| ROS1 | NM_002944 |
| RPTOR | NM_020761 |
| SDHA | NM_004168 |
| SDHB | NM_003000 |
| SDHC | NM_003001 |
| SDHC | NM_001035511 |
| SDHD | NM_003002 |
| SETD2 | NM_014159 |
| SF3B1 | NM_012433 |
| SF3B1 | NM_001005526 |
| SMAD2 | NM_005901 |
| SMAD4 | NM_005359 |
| SMARCA4 | NM_003072 |
| MYCL | NM_001033082 |
| MYCL | NM_005376 |
| KMT2A | NM_005933 |
| GID4 | NM_024052 |
| AMER1 | NM_152424 |

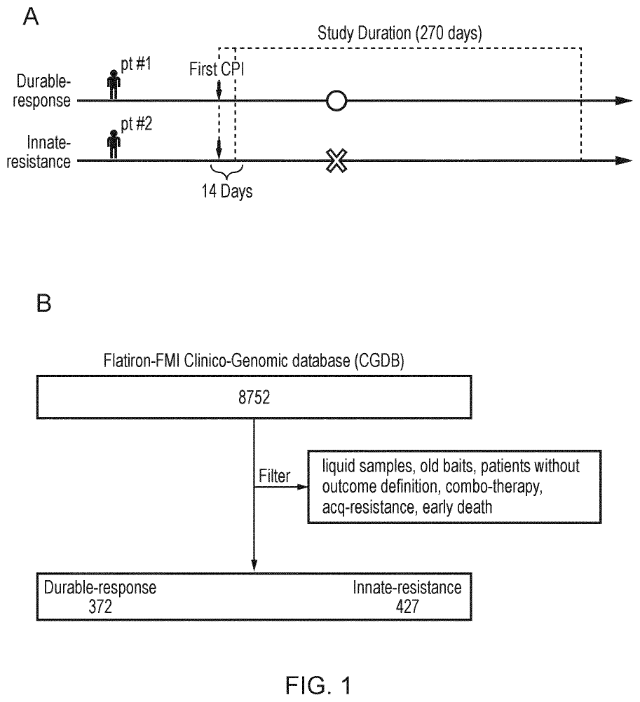

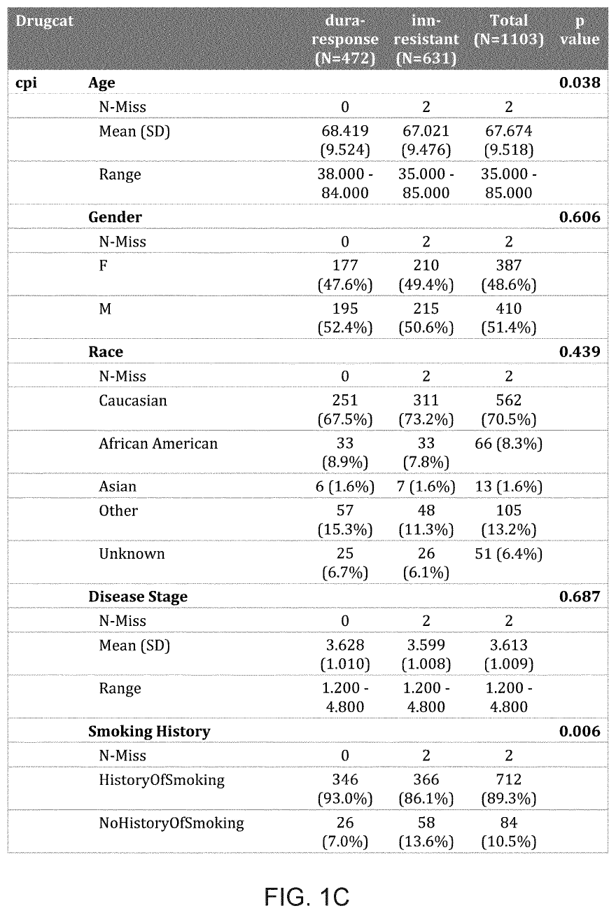

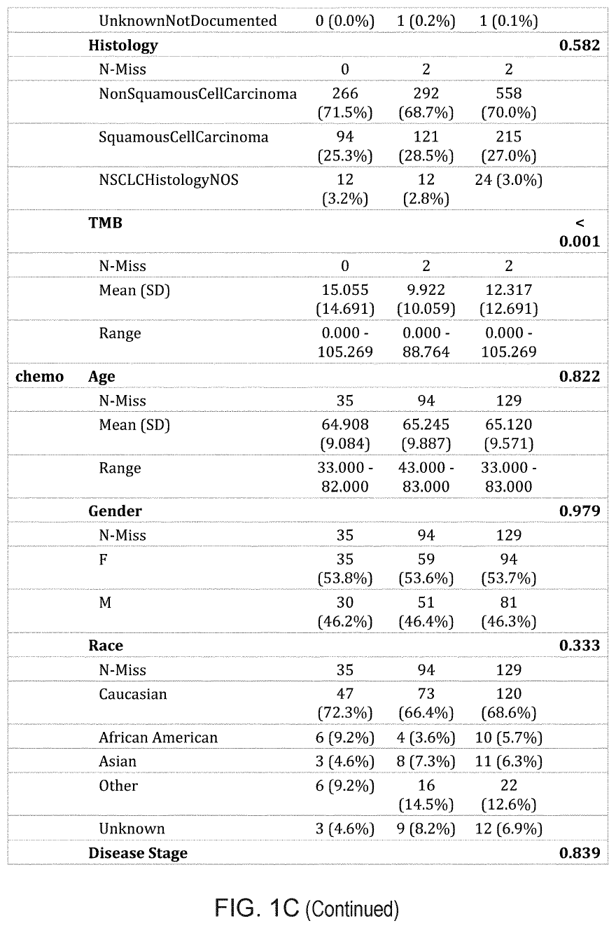

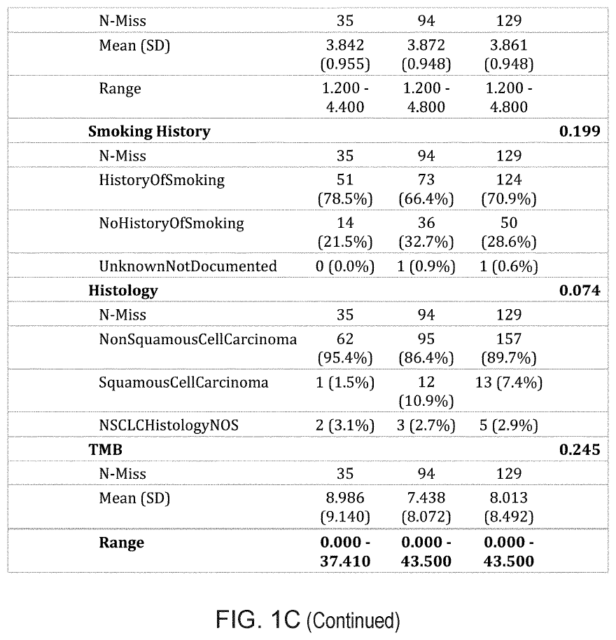

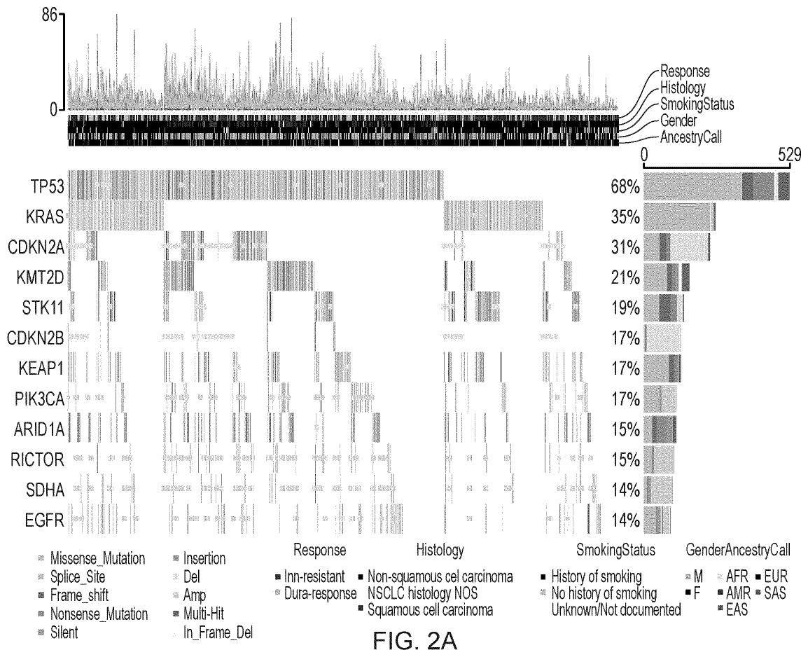

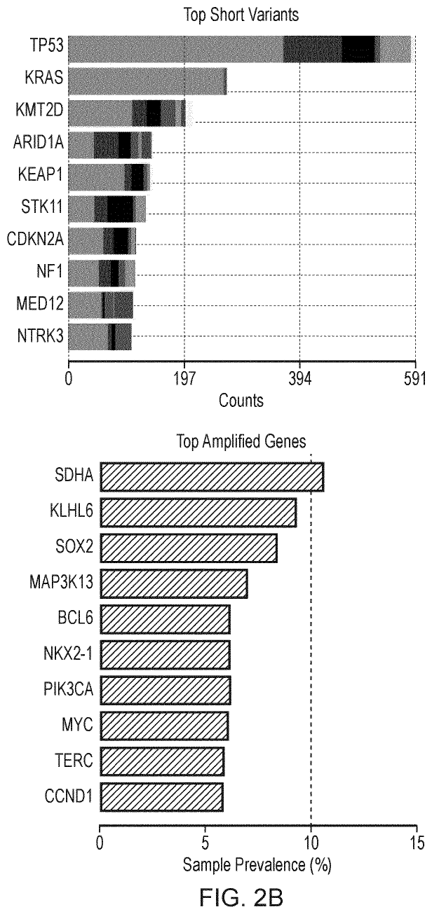

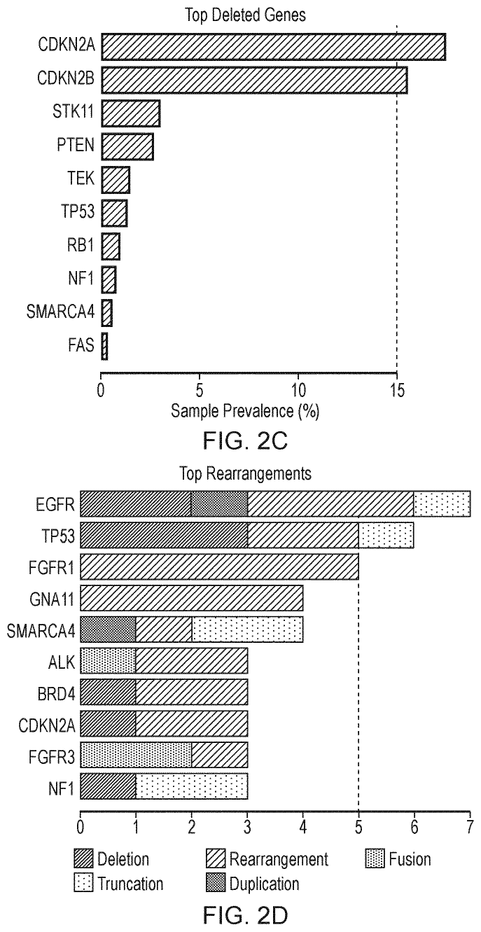

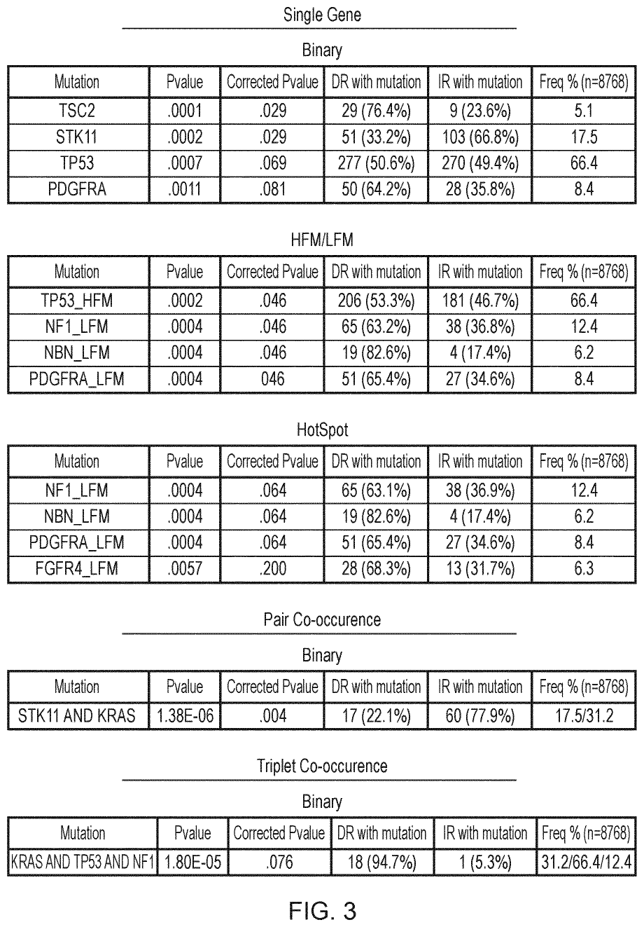

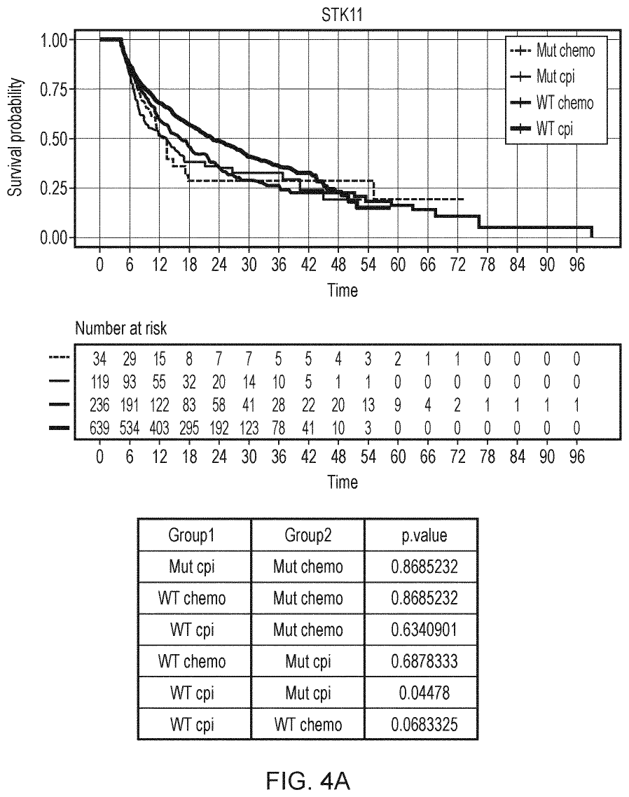

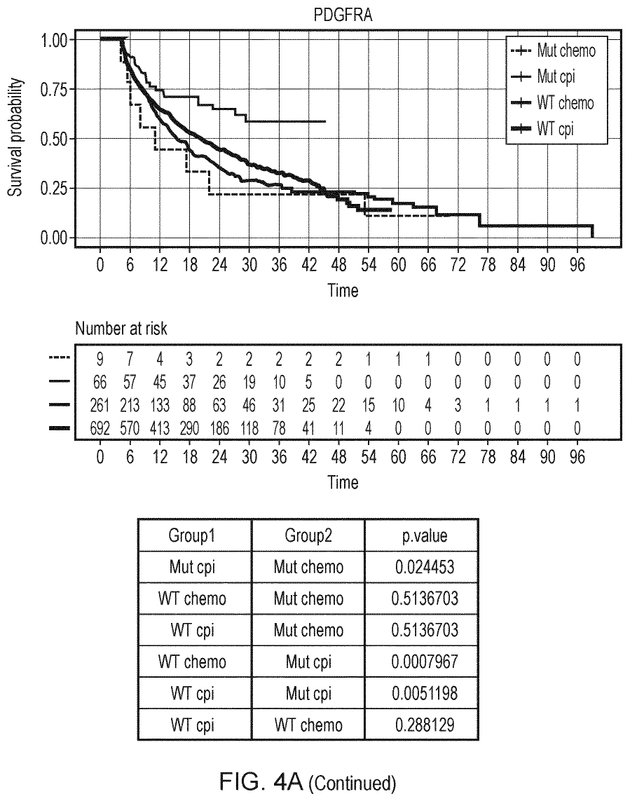

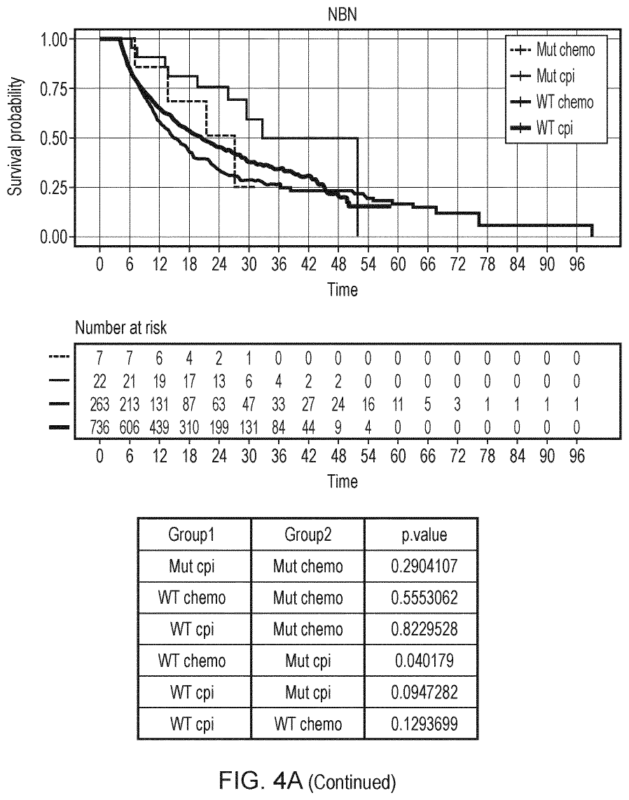

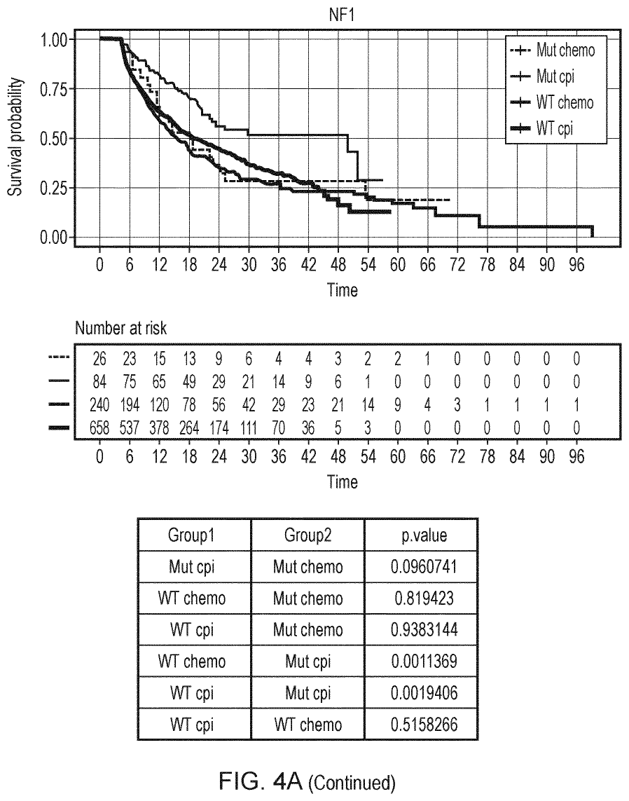

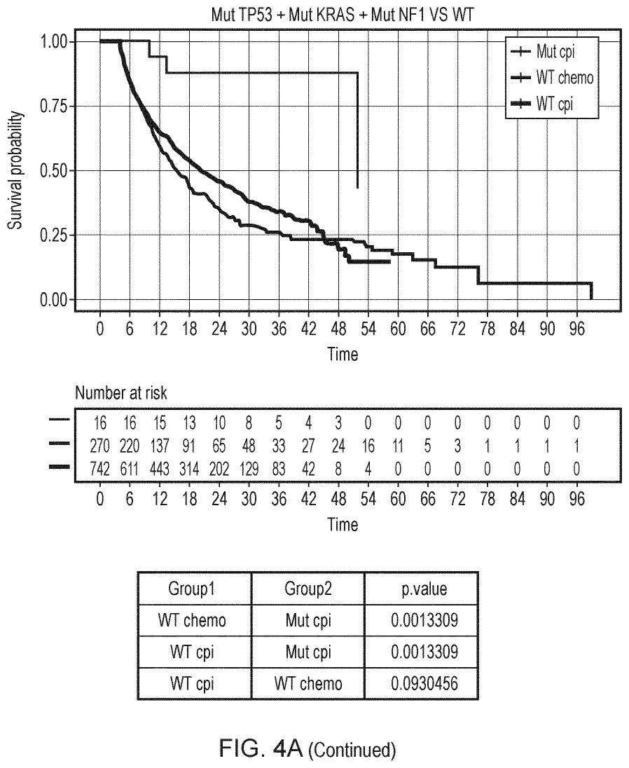

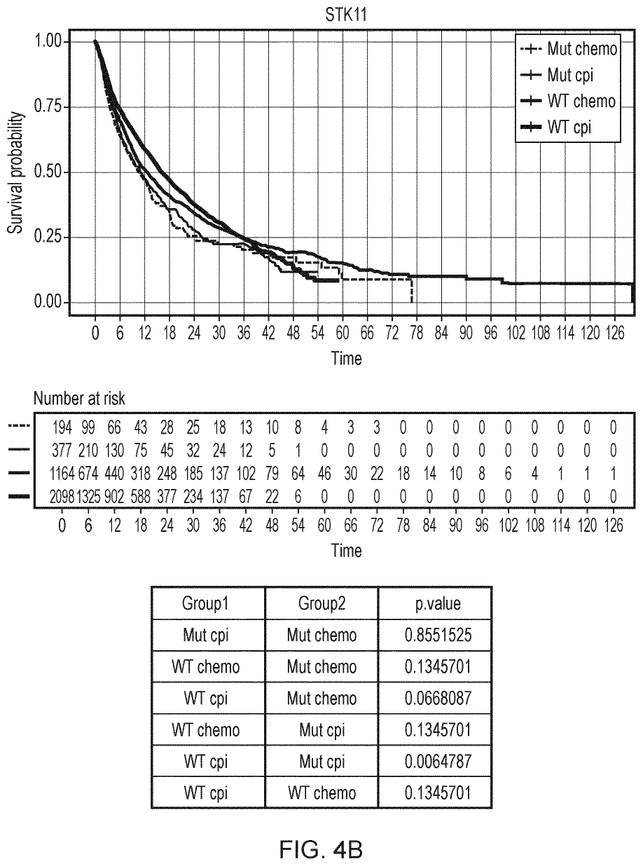

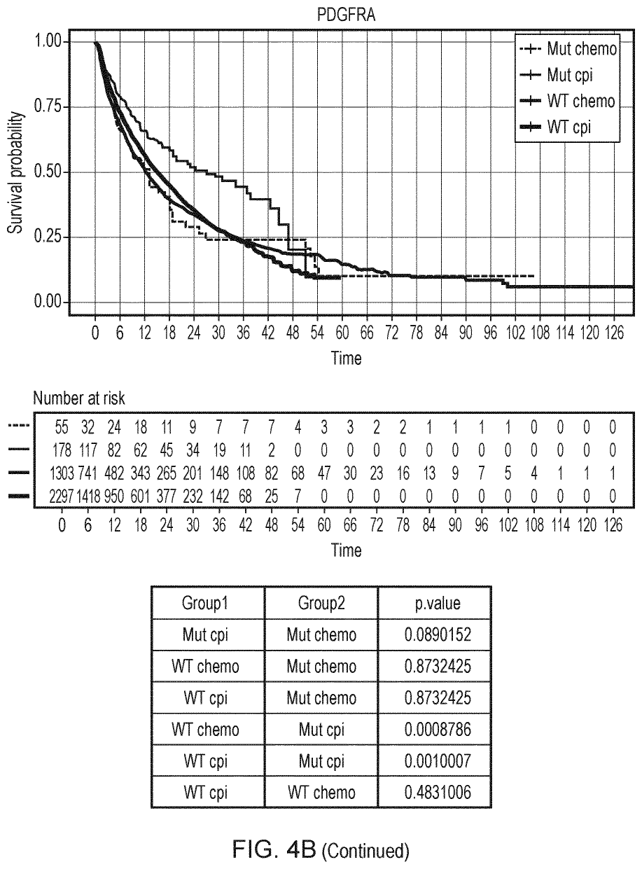

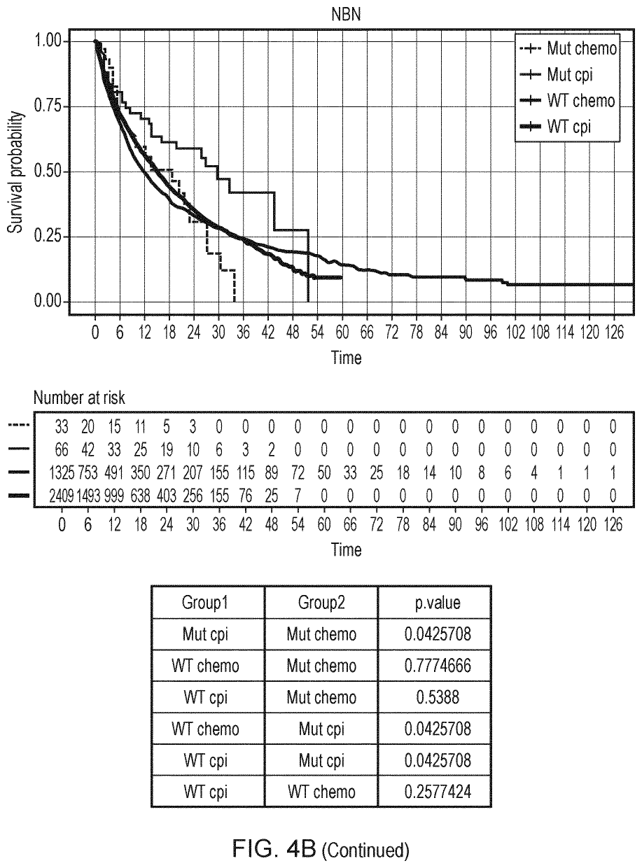

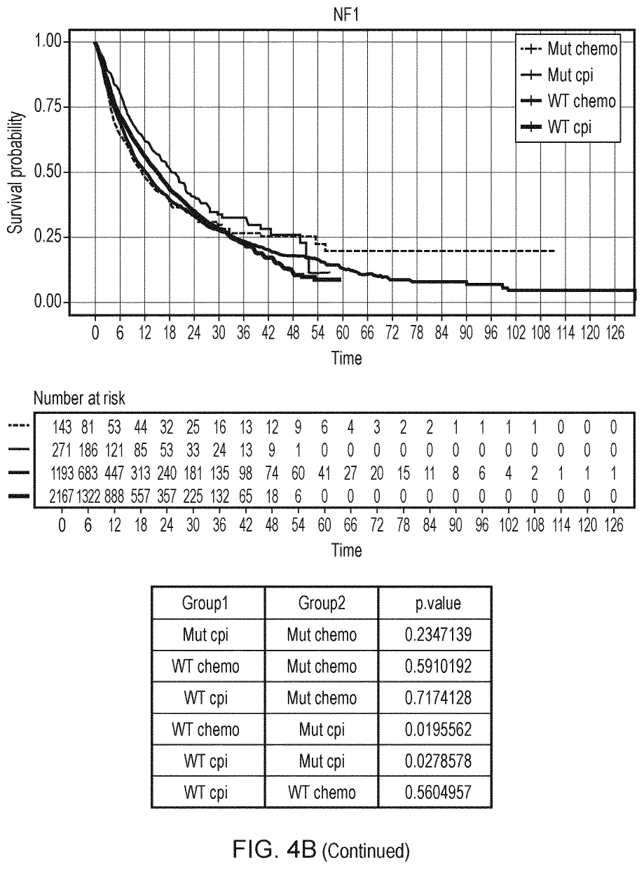

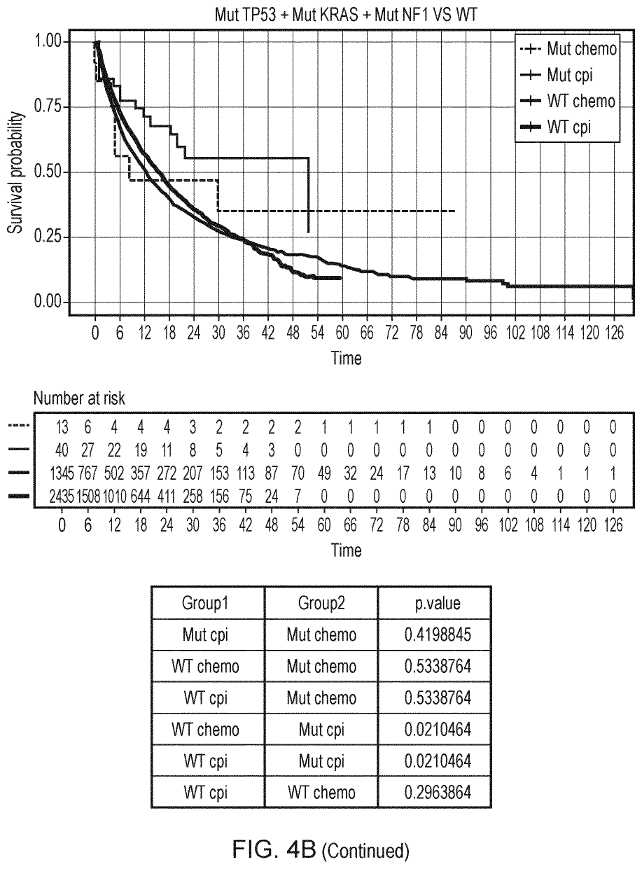

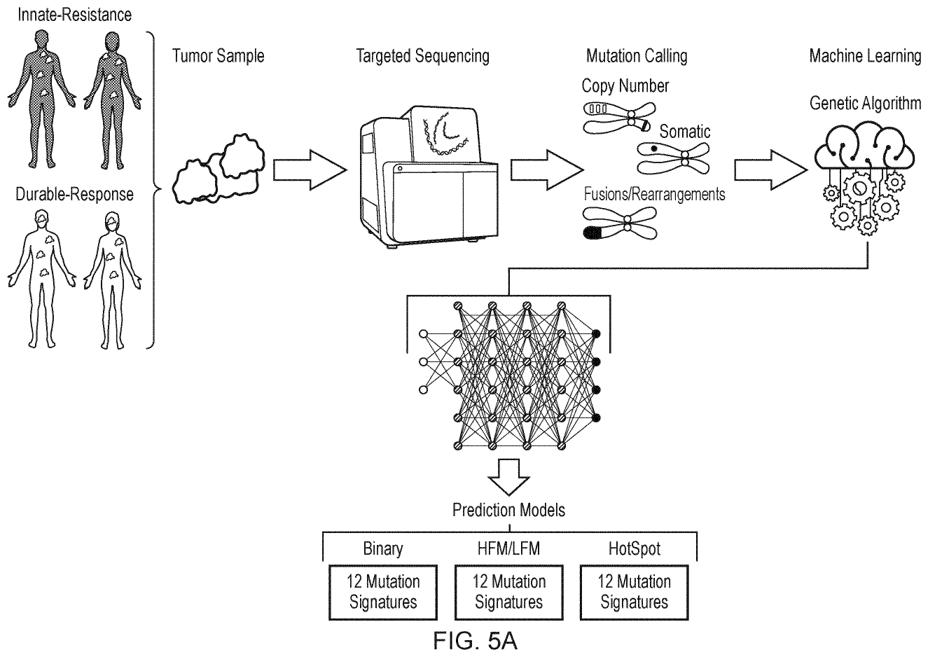

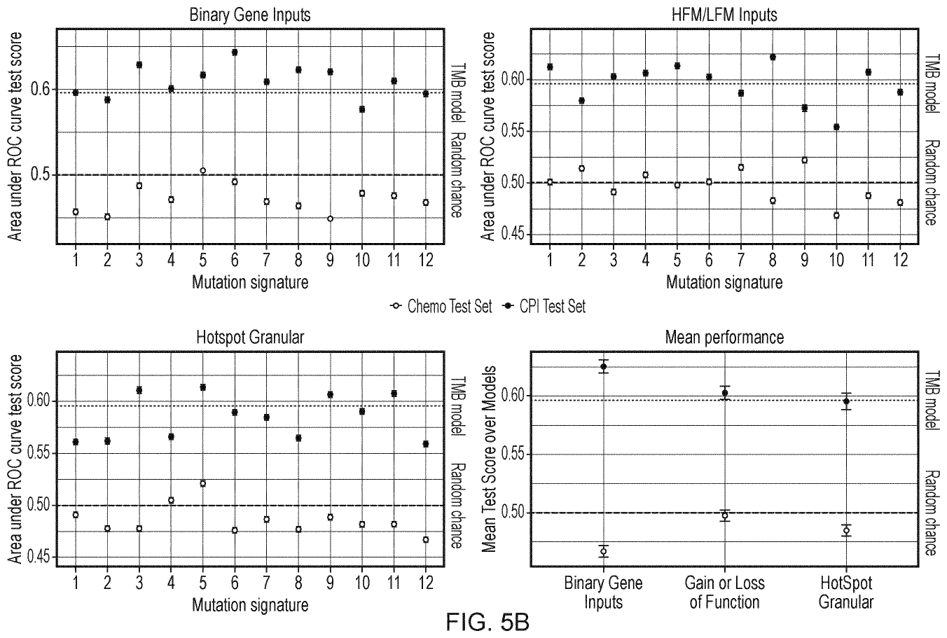

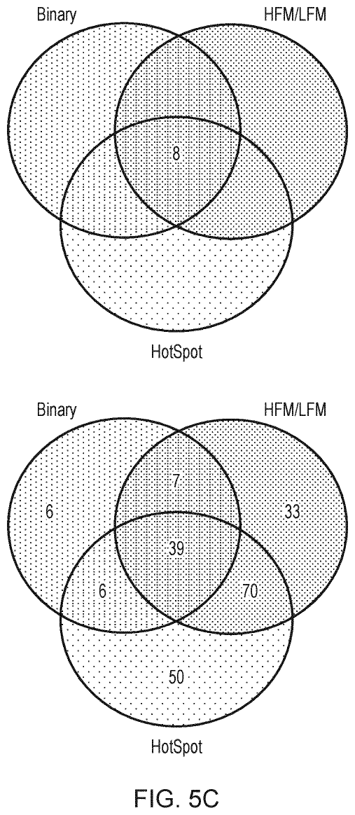

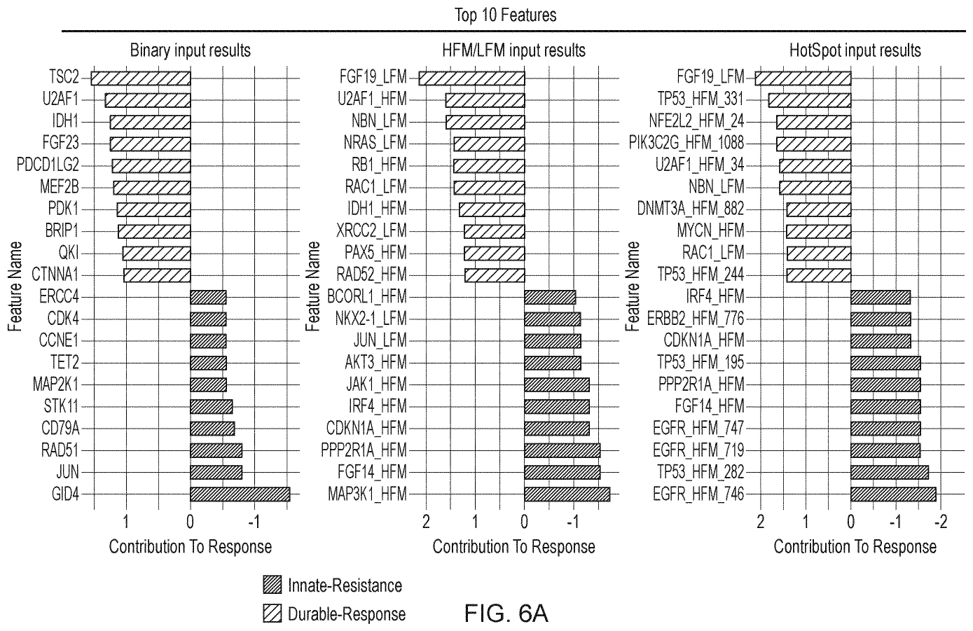

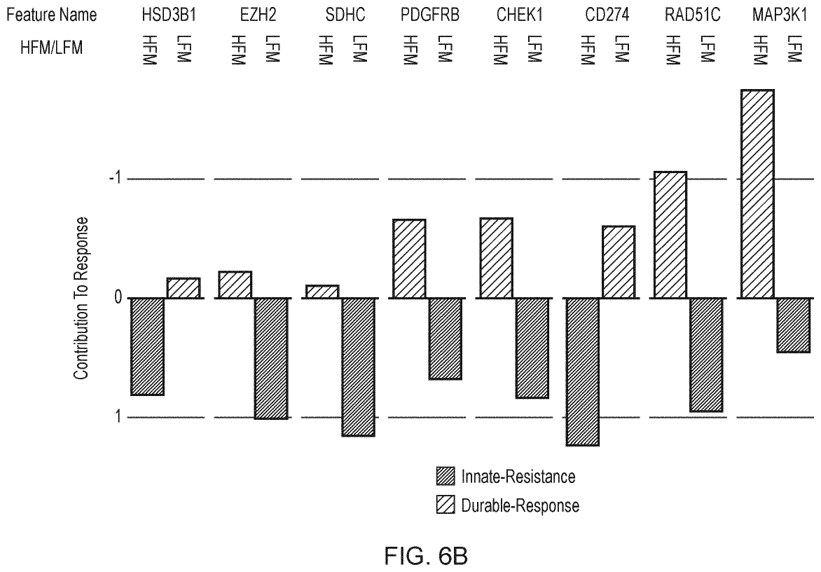

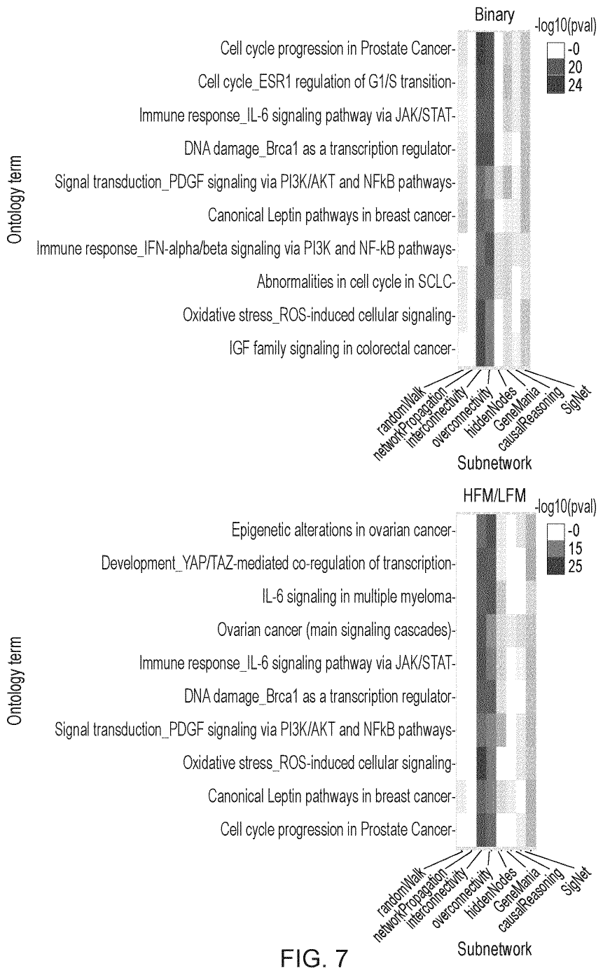

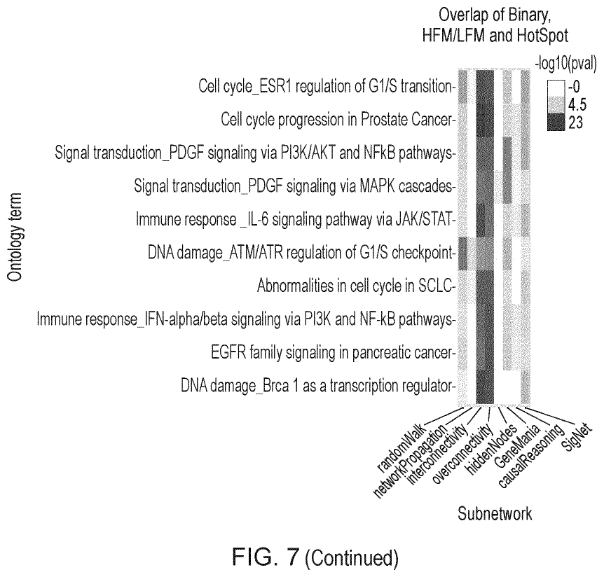

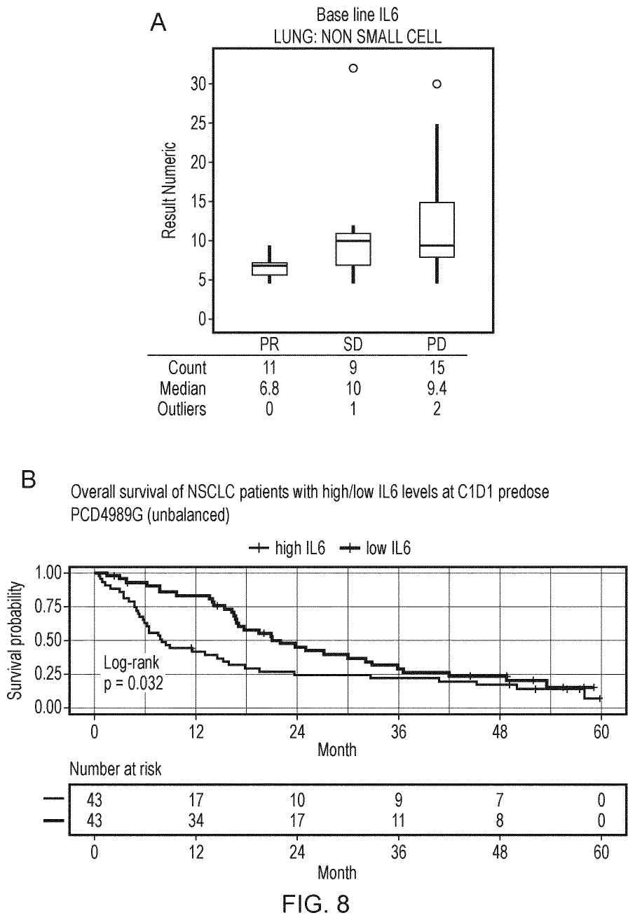

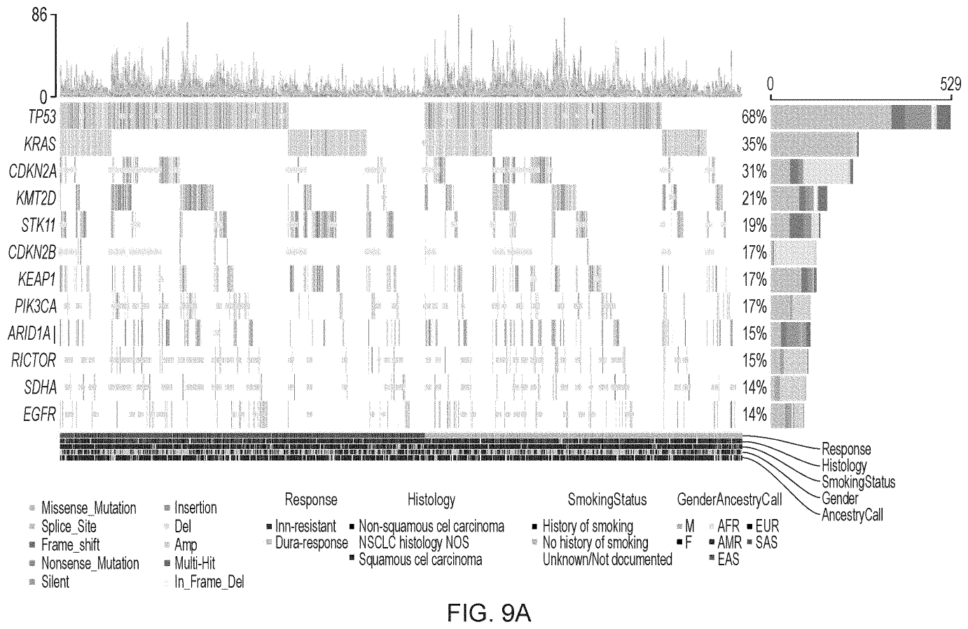

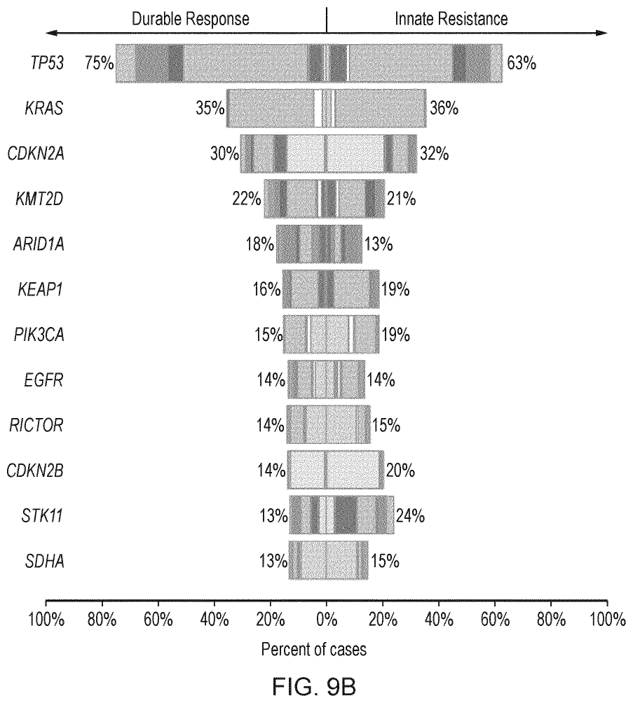

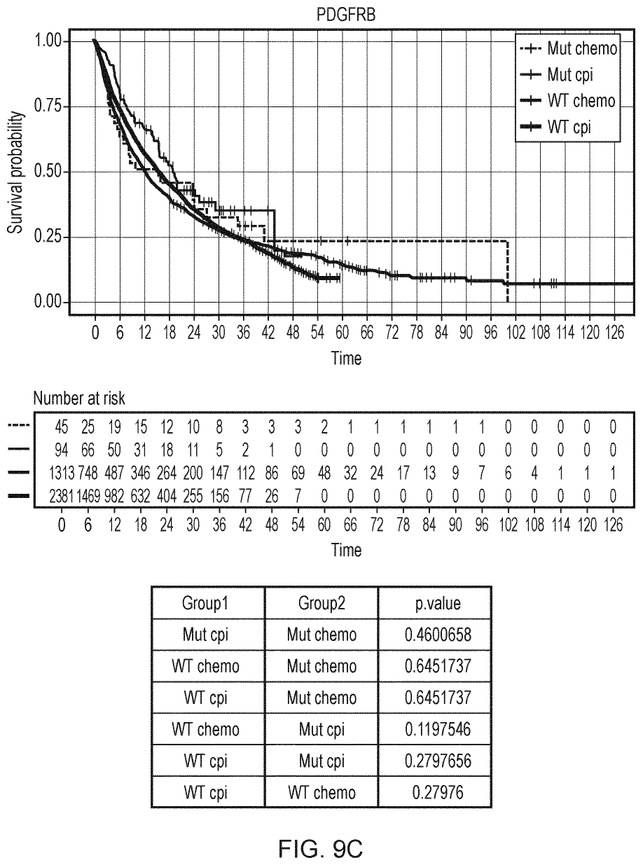

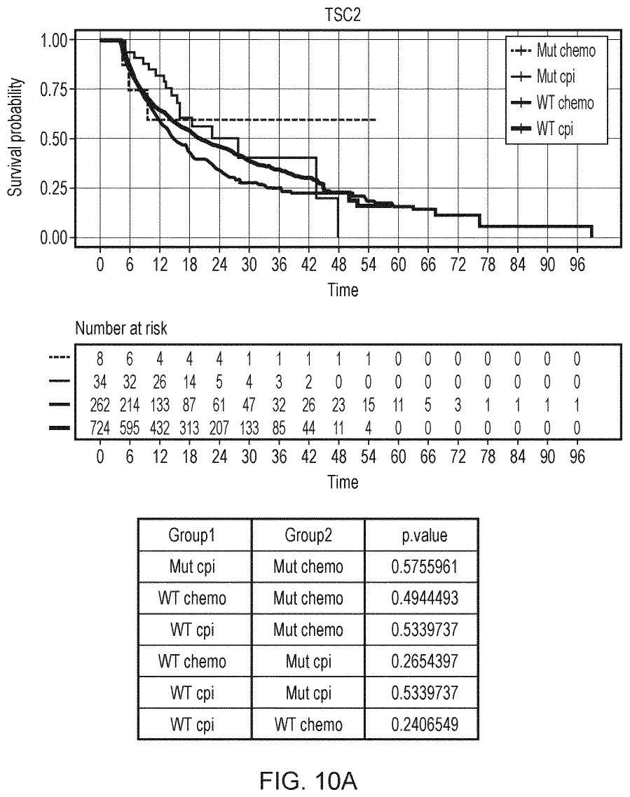

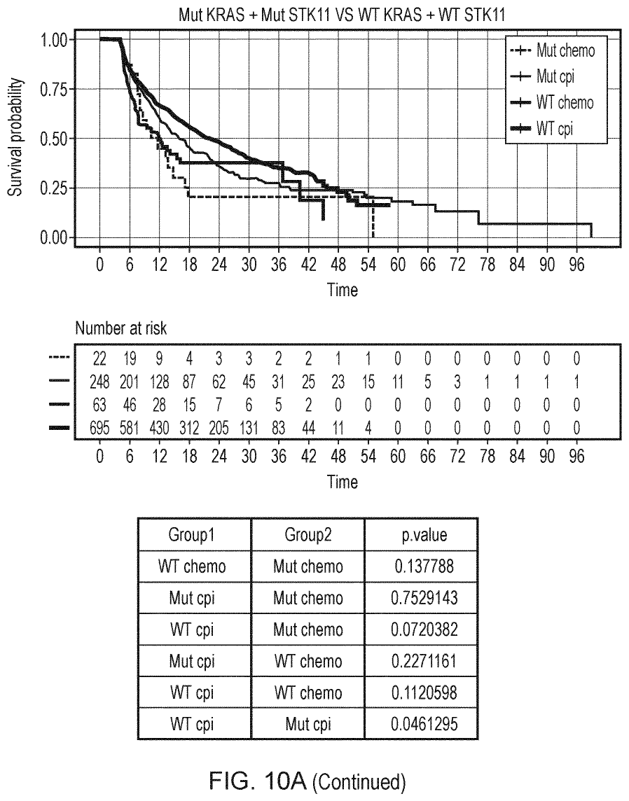

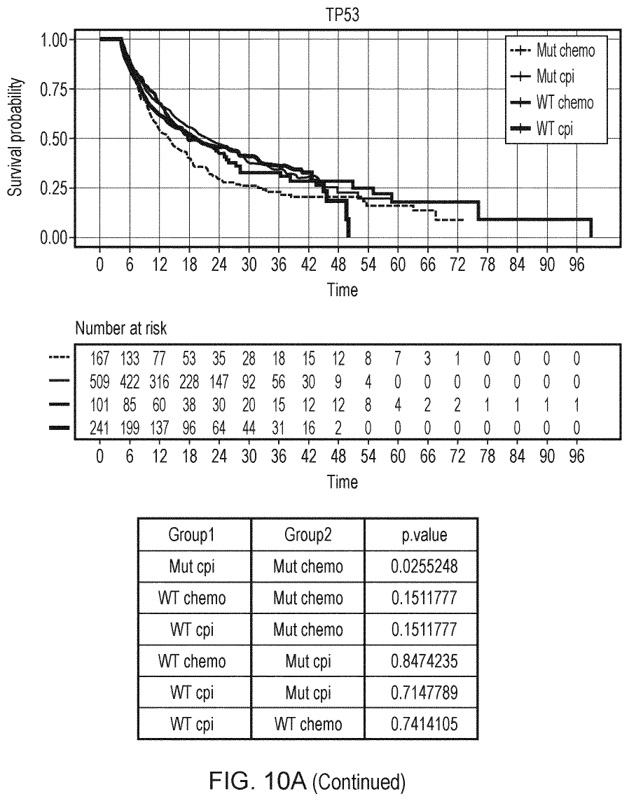

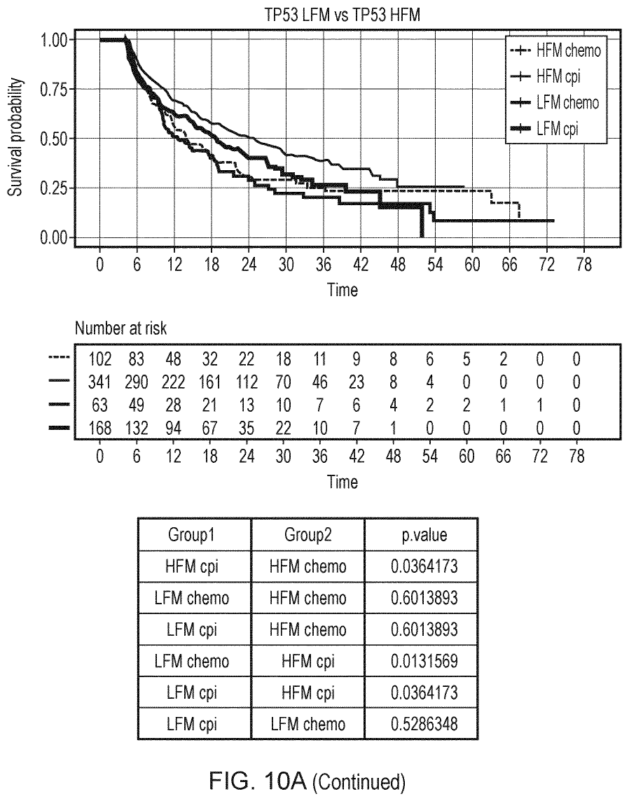

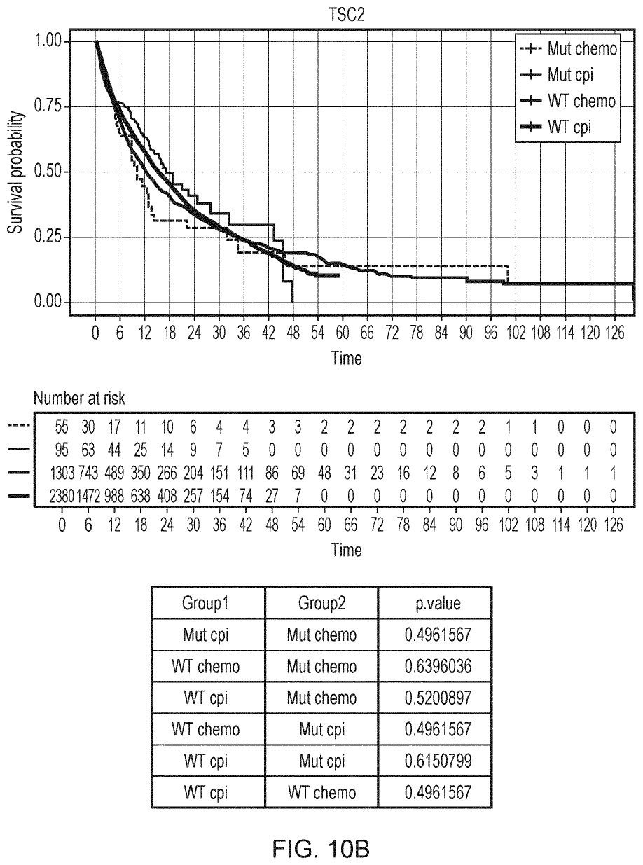

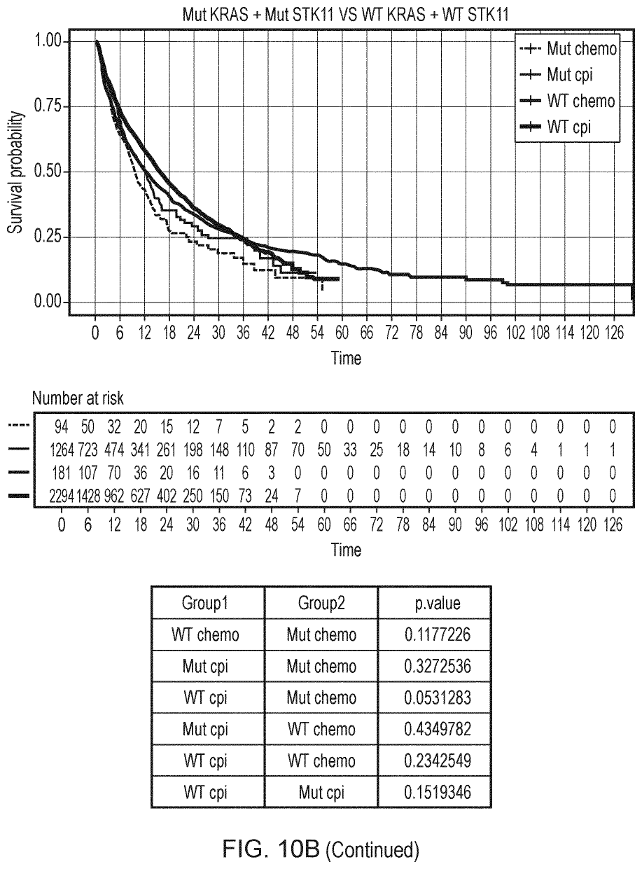

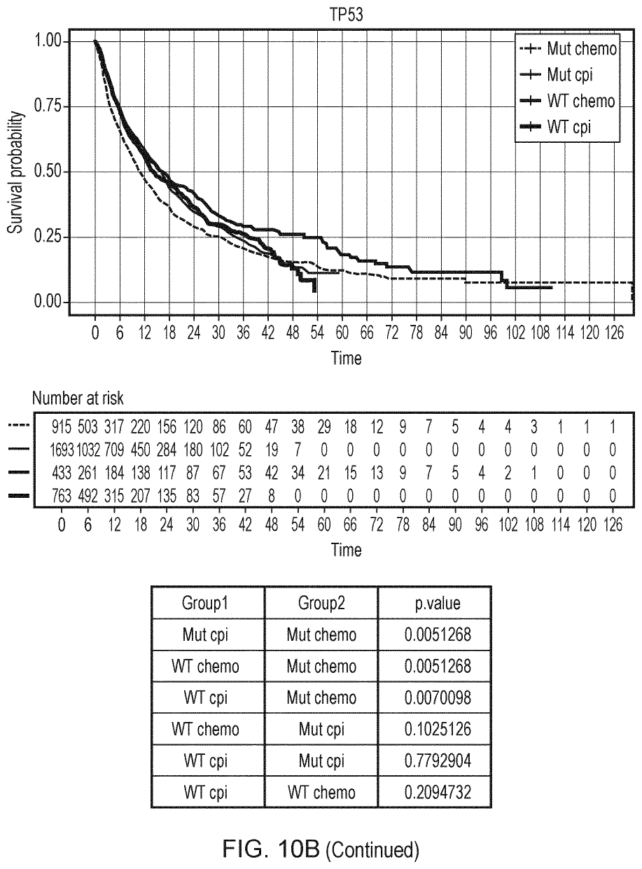

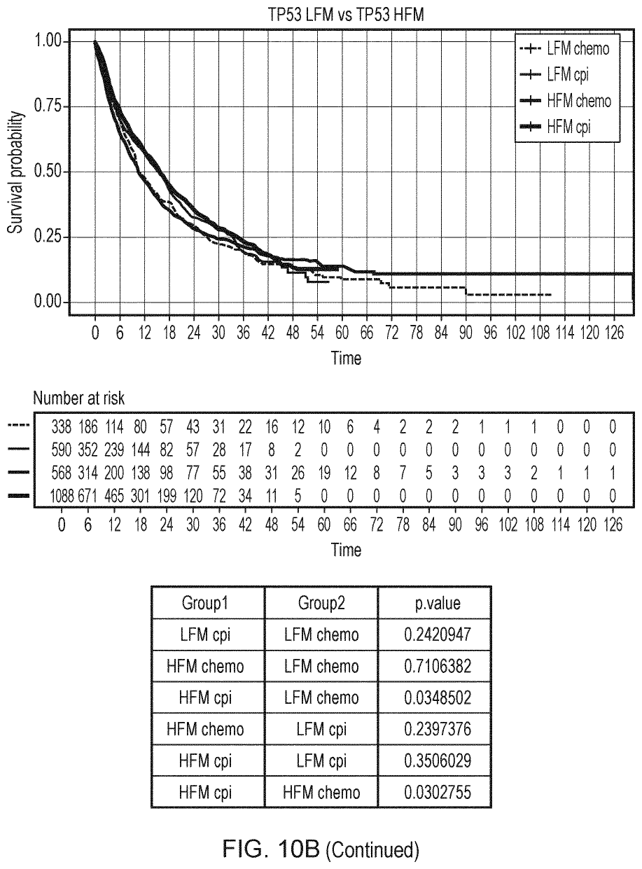

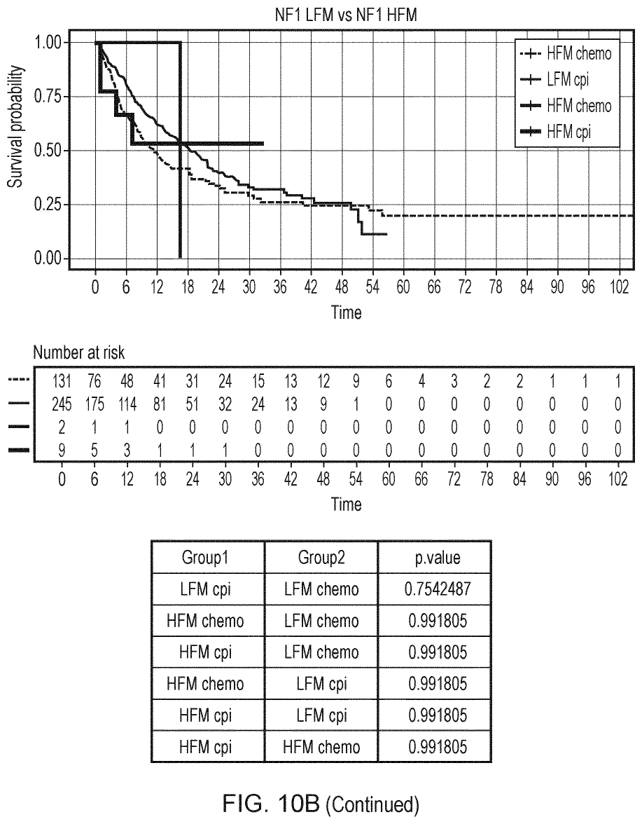

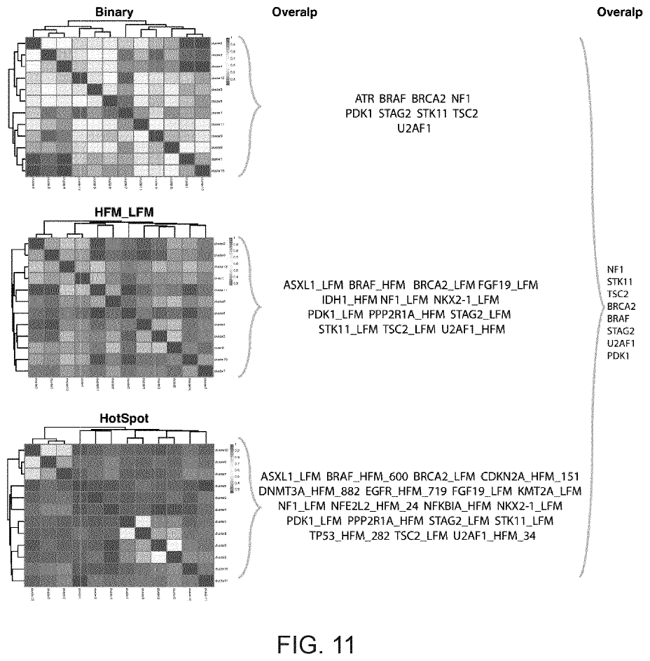

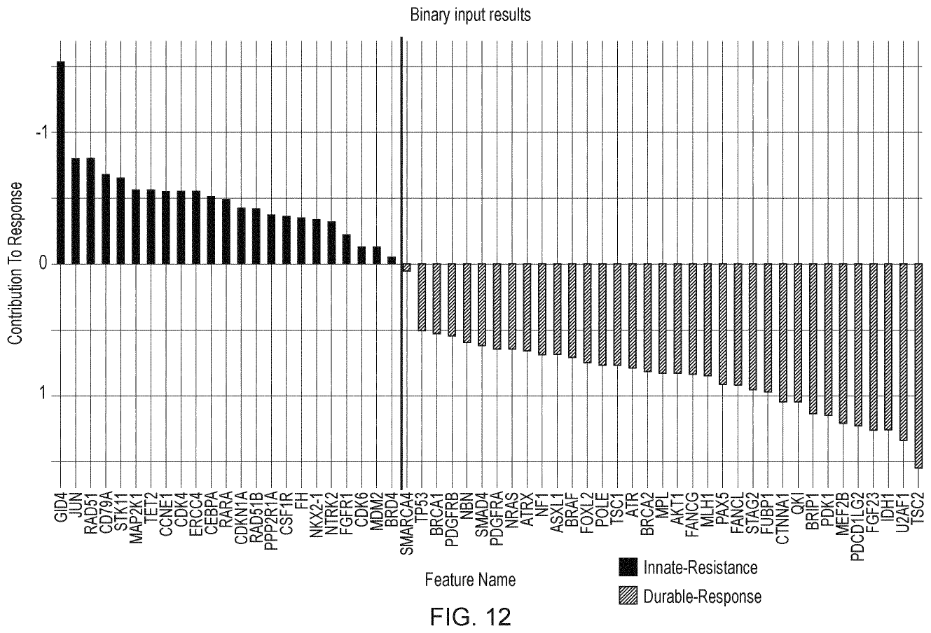

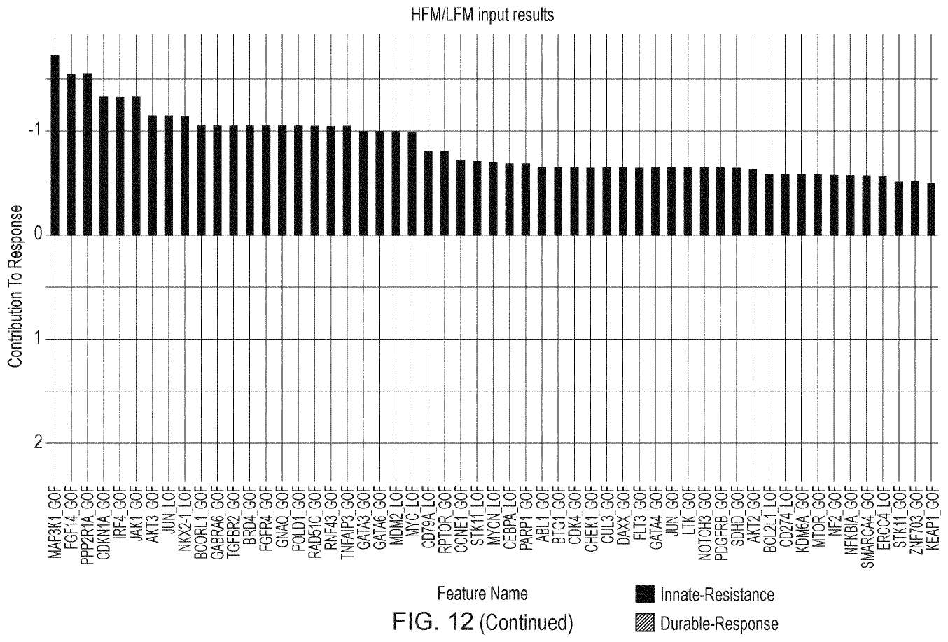

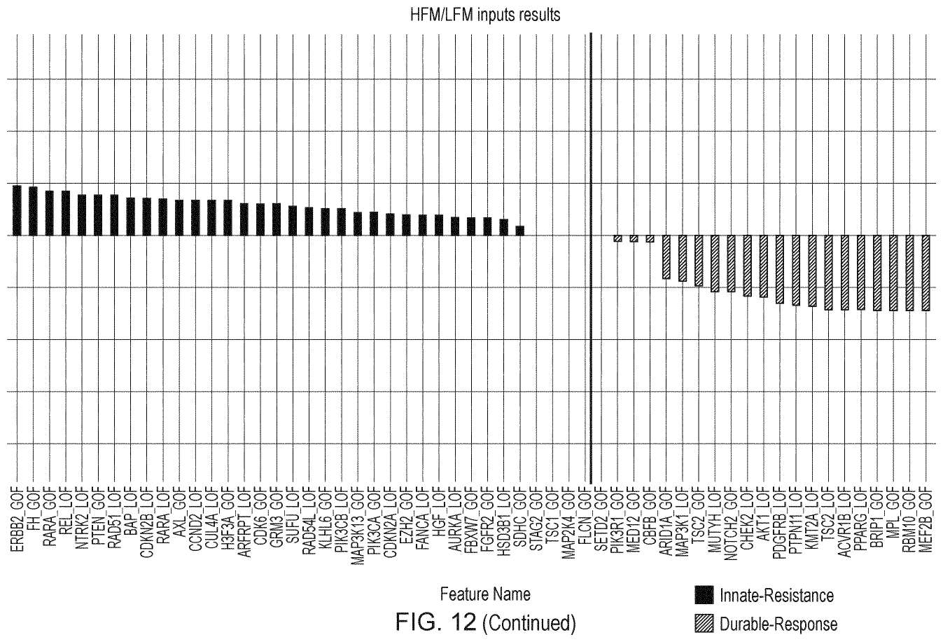

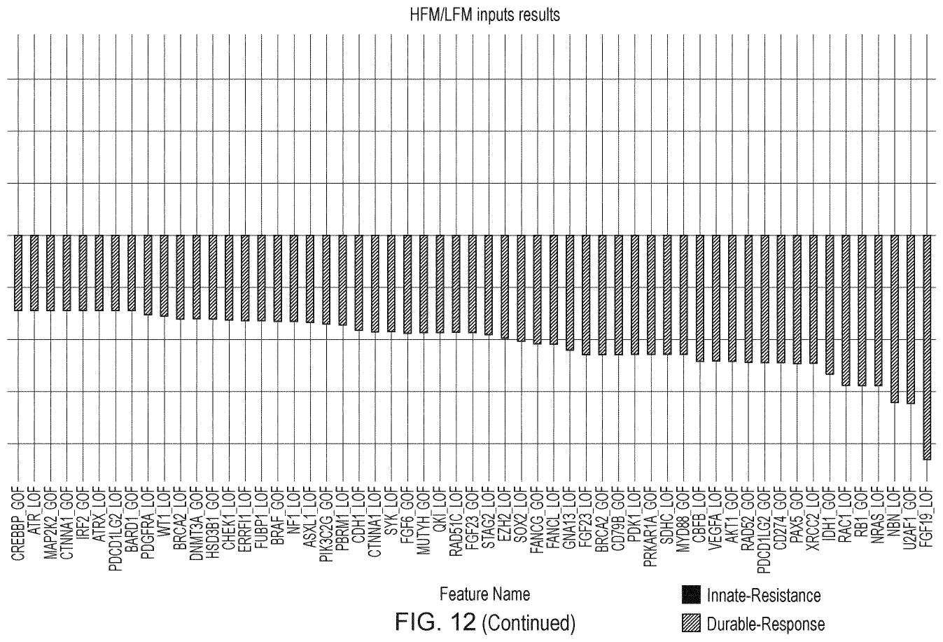

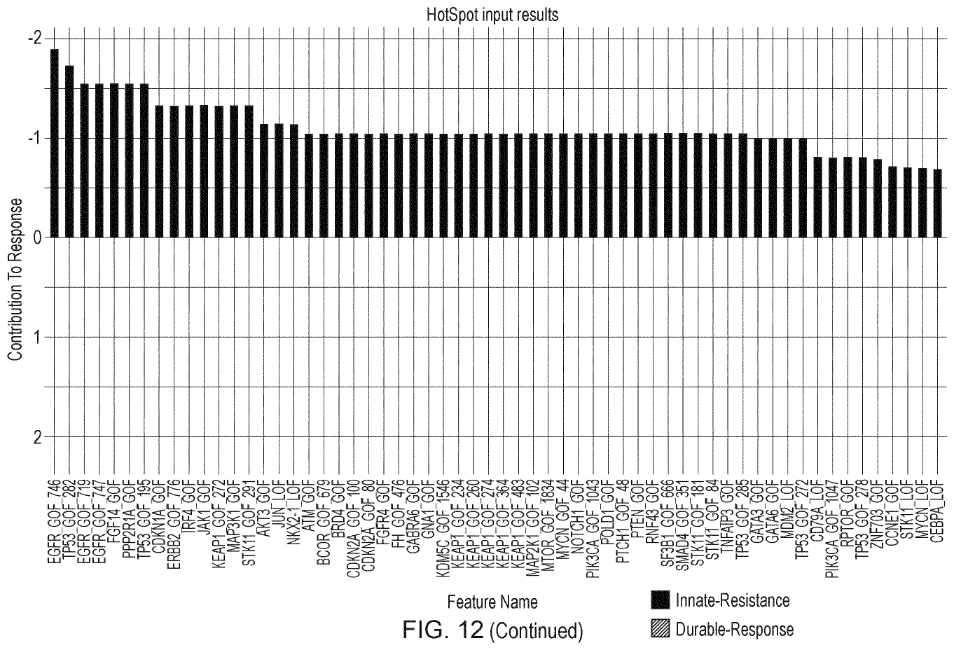

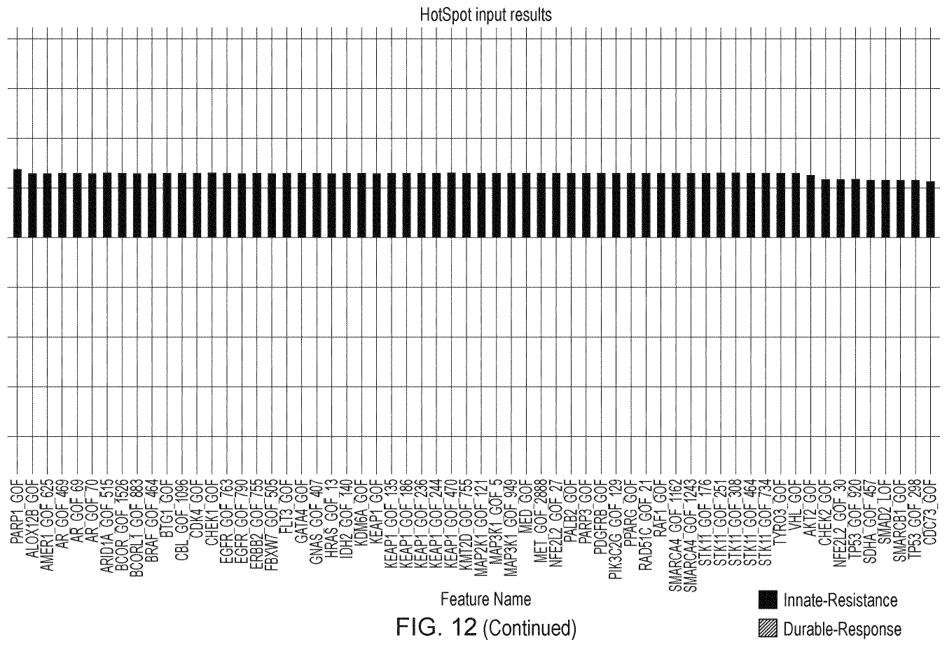

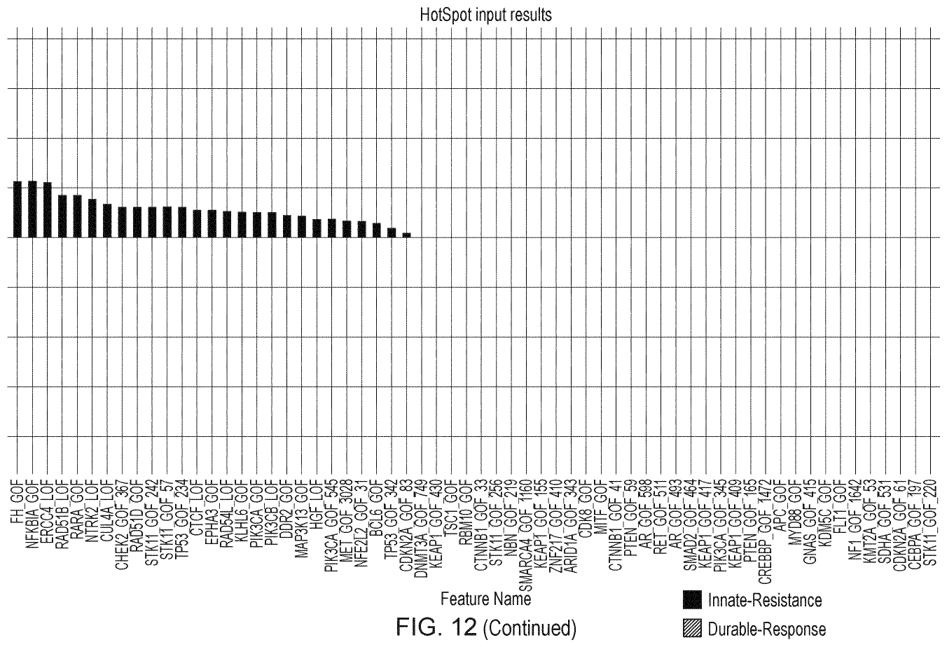

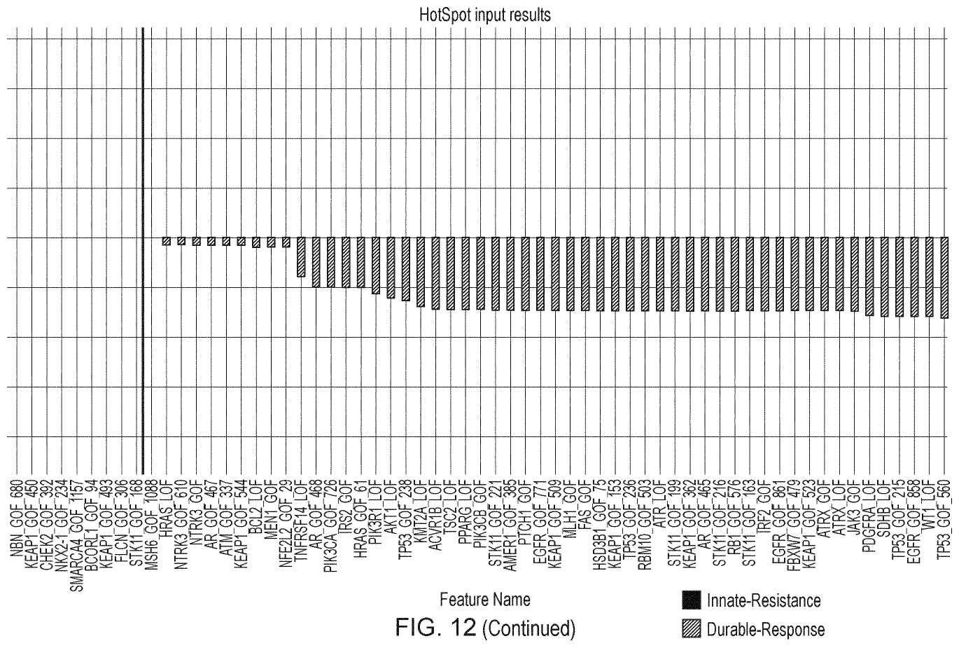

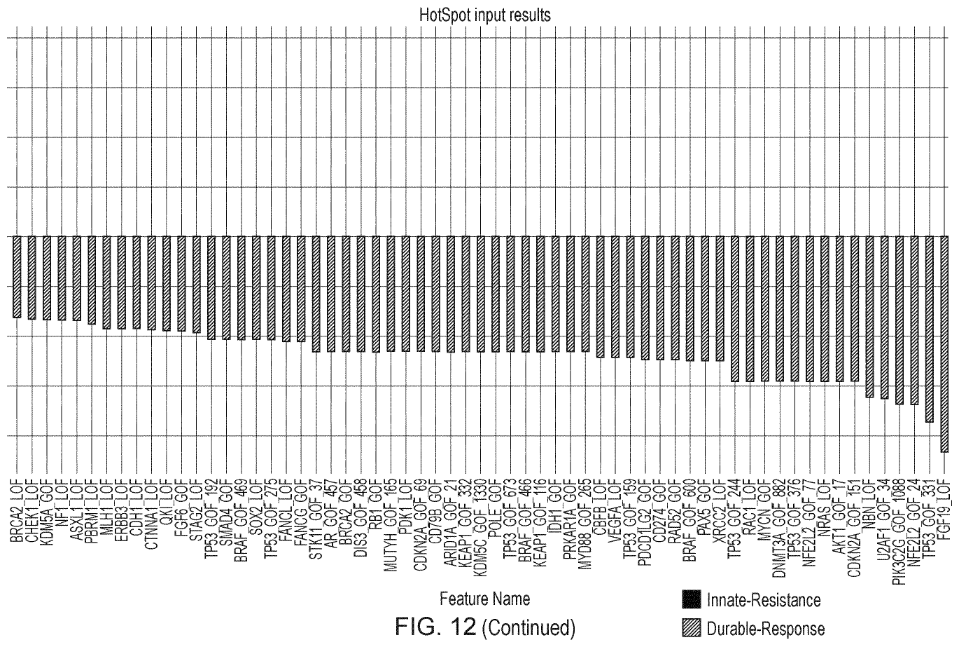

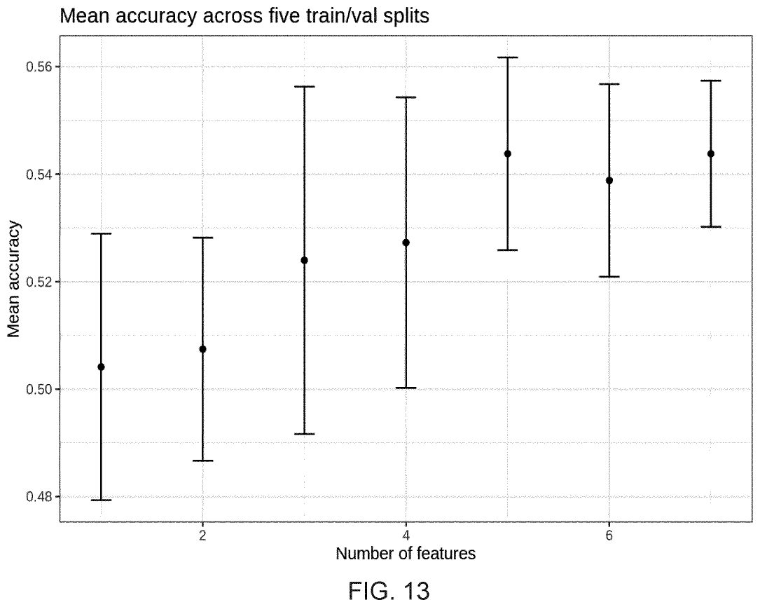

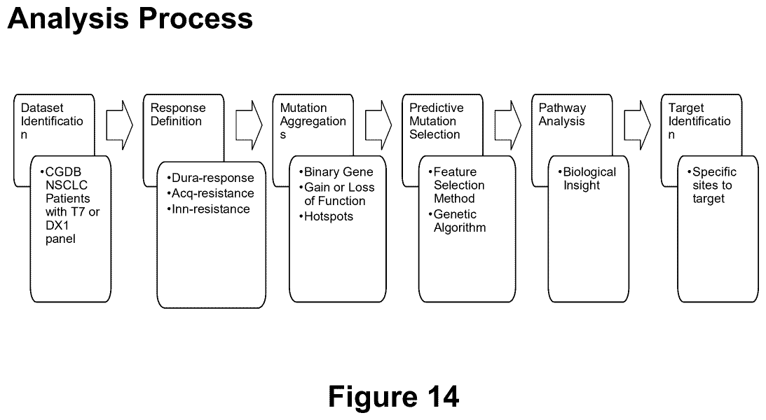

### Analysis Process (FIG. 14)

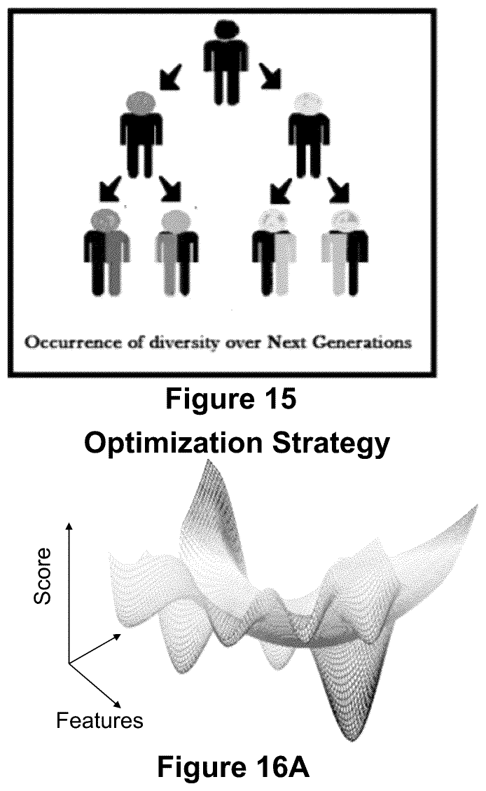

### Agenda (FIG. 15)

### Genetic Algorithms in General:

Optimization strategy with biological inspiration.
Mutations and Mating.
Maintaining diversity in the population.
   Genetic Algorithms specifically for Feature Selection:
‘Individuals’,
‘Fitness’
‘Mating’ and ‘Mutation’.

### Application on CPI Resistance:

Methods for maintaining robust solutions.

### Generic Genetic Algorithms

### Optimization Strategy

Problem: Huge number of possible solutions, how to find the best possible answer?
Try every possible solution?
   Prohibitively expensive (time or money).
   Best solution on this data might not apply to new data.
Gradient Descent?
   Not always applicable (surface is too spikey, non-numeric inputs).
   Might get stuck in local optima.

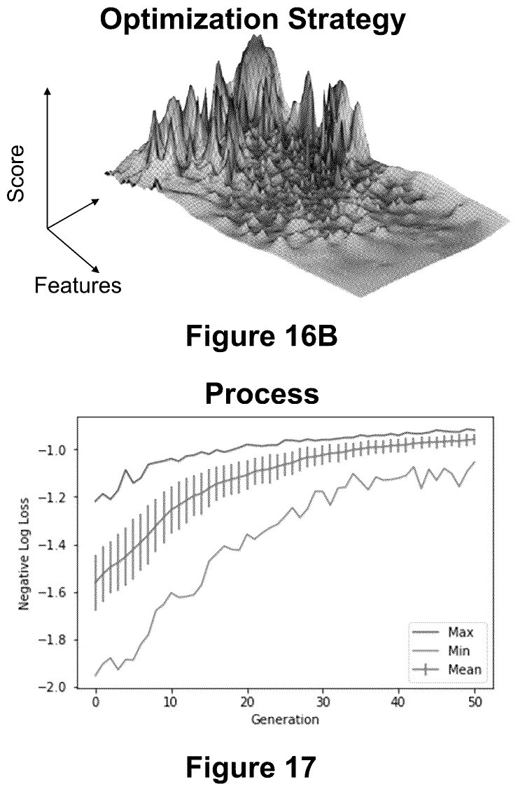

### FIG. 16A

Need: Method to find best solution out of complicated set of options.

Inspiration: genetic evolution.
Iterative process over generations.
Combine most successful previous solutions to find new solutions.
Randomly mutate previous solutions to avoid getting stuck in local optima.

### FIG. 16B

### Definitions

For each optimization problem, must organize the problem in the following ways;

Individual: One ‘solution’.
Fitness: The score by which each solution is judged.
Mutate: Method that changes one solution into a different solution.
Mating: Method that combines two solutions into a new, different solution.

Concrete Examples: linear regression mX+b on a fixed data set.
Individual: One set of values for m and b.
Fitness: MSE of the residual of the line mX+b on the dataset.
Mutate: Randomly add/subtract values to m and b.
Mating: Average the m and b values between the two parents.

Process (FIG. 17): 1. Generate the first Generation of Individuals. 2. Evaluate the Fitness of every Individual. 3. Choose the best Individuals. 4. Mate and Mutate the best Individuals to form a new generation. 5. Repeat steps 2-4 until ‘done’.

### Genetic Algorithms for Feature Selection

### Feature Selection:

Feature Selection is usually a pre-processing step to ‘canonical’ modelling.
   Increase signal-to-noise ratio.
   Reduces over-training.
   Improves statistical power.
Can be informative on its own.
   Some problems only require knowing which features are important, not maximizing their predictive potential.
In our case, features=mutations.

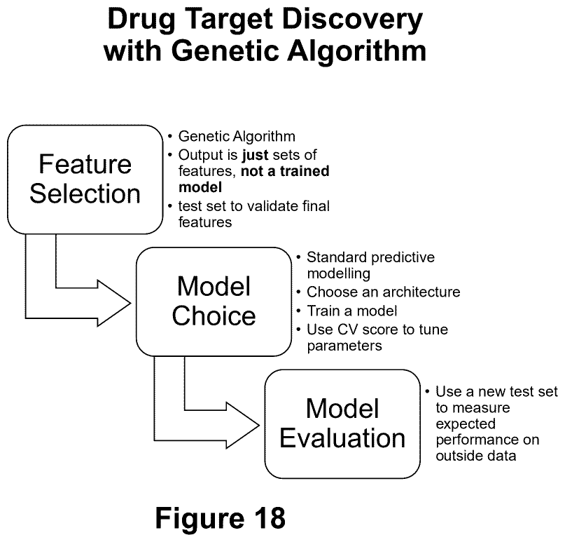

FIG. 18 shows drug target discovery with genetic algorithm. Current analysis is focused on exploring the data, identifying important targets. The current set is used to validate the features, not optimize model parameters. You only need the features for target discovery—a fully optimized predictive model is not necessary.

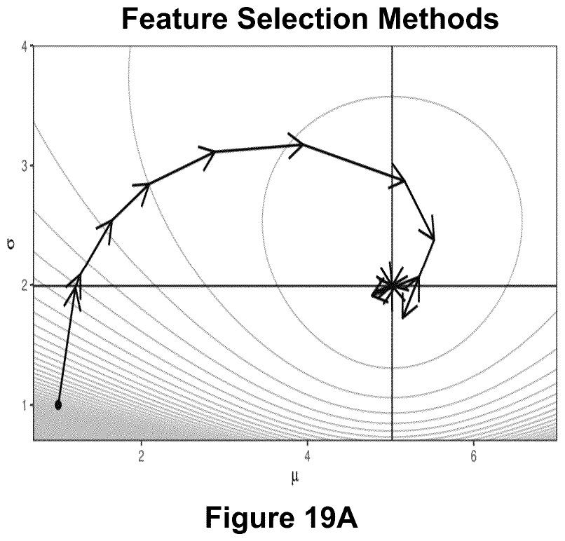

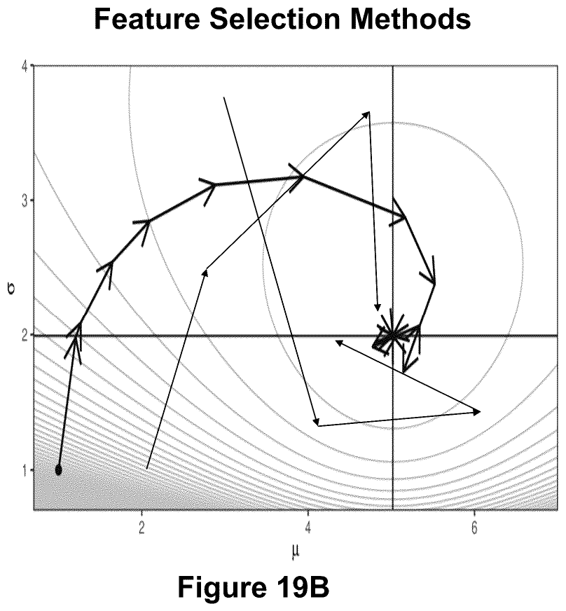

### Feature Selection Methods (FIG. 19A and FIG. 19B):

Univariate Selection:
   Choose features that are already statistically significant on their own.
   Cannot account for feature interactions.
Recursive Feature Elimination (RFE) or Sequential Feature Selection (SFS):
Iteratively build feature sets which improve a reference model.
Can only find feature combinations that are additive/subtractive.

### FIG. 19A

Genetic Algorithm:
   Evaluates groups of features (can include interaction terms).
   Score is based on model performance for a reference model.
   Can escape local optima through diversity of genetic individuals.
   Simultaneously evaluates many optimization paths (easily parallelizable).

### FIG. 19B

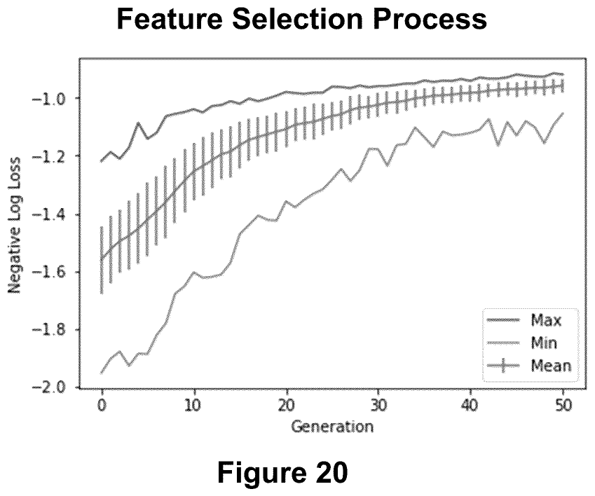

Feature Selection Process (FIG. 20): 1. Randomly generate a first generation of feature sets (individuals). 2. Evaluate the accuracy of a reference model using each of the individuals' features. 3. Choose the best performing sets of features (individuals). 4. Mutate/Mate to form a next generation. Repeat steps 2-4 until fitness score convergence.

### CPI Resistance

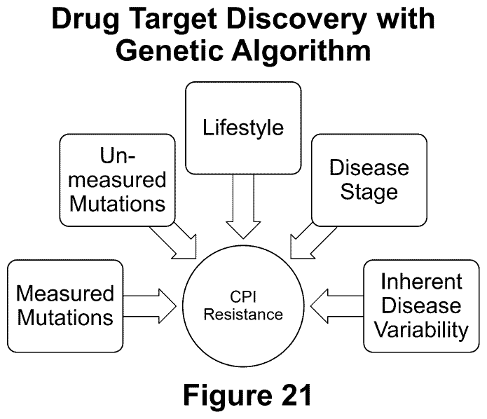

Drug Target Discovery with Genetic Algorithm (Table a and FIG. 21)
Dataset:
   ‘Rows’ are ˜1000 NSCLS Patients;
   ‘Columns’/features=mutations from FMI panel on tumor.
      The mutations are categorized several ways.
      Number of possible ‘features’ range from 285 from simplest categorization to 2000 for most complicated categorization.
   Target: CPI drug resistance, defined by progression of patient.
Goal:
   Find mutations which are predictive of CPI resistance

TABLE A

| | | | | | | | |
|---|---|---|---|---|---|---|---|
| PatientID | ABL1 | ACVR1B | AKT1 | AKT2 | AKT3 | ALK | AL |

| | | | | | | |
|---|---|---|---|---|---|---|
| E9FFFD06 | 0 | 0 | 0 | 0 | 0 | 0 |
| D8BB110B | 0 | 0 | 0 | 0 | 0 | 0 |
| 3EA6D7E | 1 | 0 | 0 | 0 | 0 | 1 |
| 09B253A7 | 1 | 0 | 0 | 0 | 0 | 0 |

Data Science Difficulties.
   High noise floor:
      Definition of ‘response’ is non-trivial.
      Inputs are not expected to explain all outcomes (only 285 genes measured).
   Wide dataset:
      Number of features exceeds the number of rows-very hard to avoid over-training.
      Combinatorial explosion of feature combinations (2\hat{ }285=10\hat{ }85 combinations).

### FIG. 21

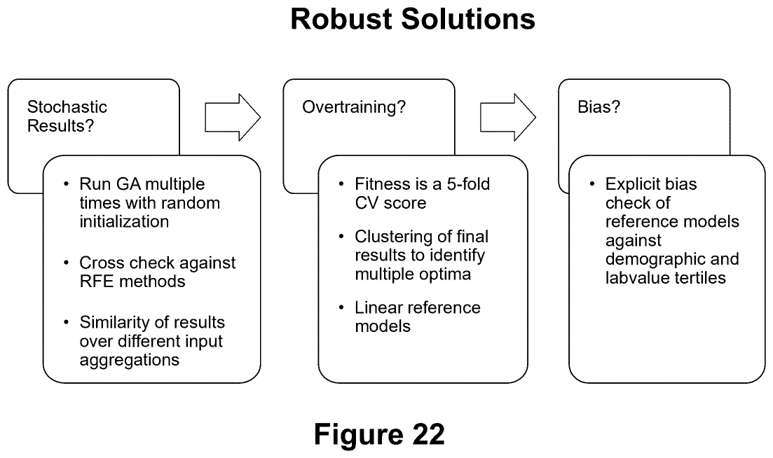

Robust solutions are shown in FIG. 22.

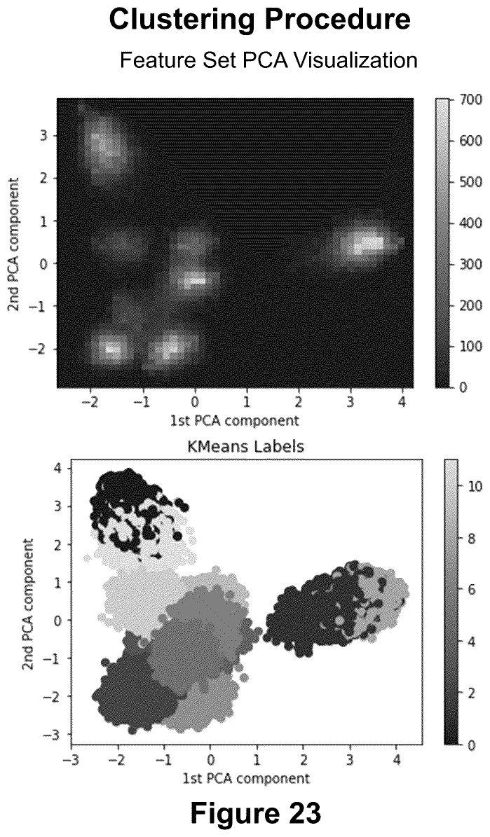

### Clustering Procedure (FIG. 23)

Single GA individual/feature set may be fluke.
Neighborhood of well-performing feature sets likely to mean robust maxima.
Motivates clustering procedure:
   All feature sets with high CV score are clustered;
   Most common features in those clusters are judged on test set;
      Well-performing cluster-sets form output of algorithm.

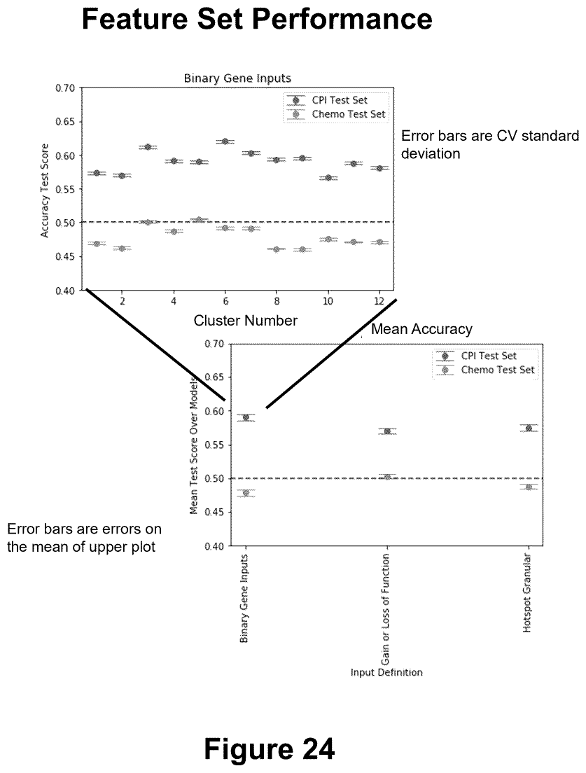

### Feature Set Performance (FIG. 24):

The final feature sets achieve a prediction accuracy of roughly 59% on a held-out CPI test set.
On a control test of chemo patients, prediction accuracy is consistent with random change.
   Implies prediction is based on CPI resistance, not prognosis.

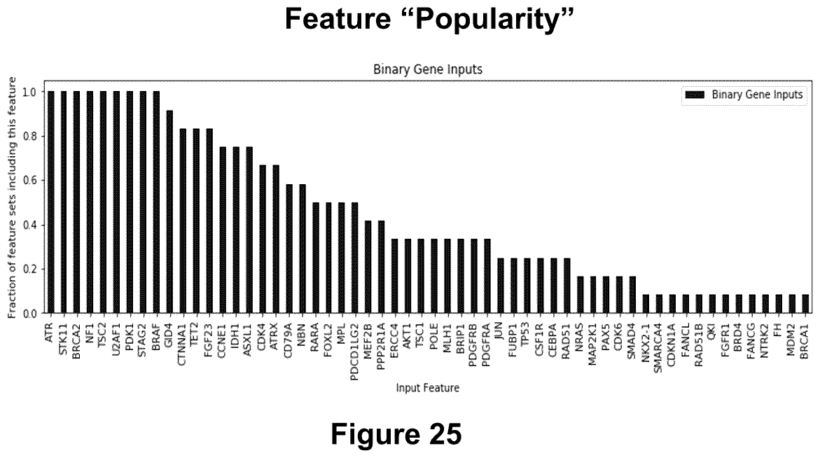

### Feature “Popularity” (FIG. 25):

Features that only appear in one or two clusters may be spurious.
Features that show up in most clusters are consistently important for prediction.
Therefore, more popular features across well-performing clusters indicate consistent predictive power.

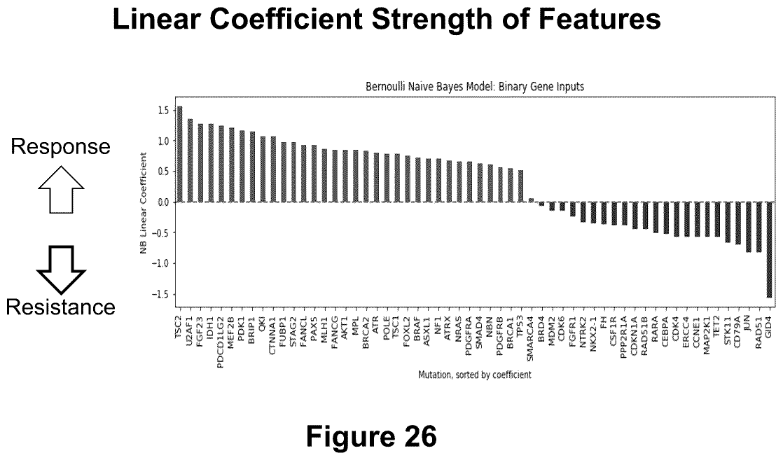

### Linear Coefficient Strength of Features (FIG. 26):

Features are selected using Naïve Bayes model to measure predictive power.
Naïve Bayes models have equivalent linear formulations.
Therefore, linear coefficient size of features can be calculated, shows the relative univariate predictive power of features.

### Genetic Algorithm for Feature Selection

Individuals.
   Sets of features.
   Defined in-code as a binary mask of features.
Example
   Individual 1 uses only features “AA”, “BB”, “EE”, and “FF”.
   Individual 2 uses only features “AA”, “BB”, “CC”.
   Table B.

TABLE B

| Feature | Individual 1 | Individual 2 |
|---|---|---|
| AA | 1 | 1 |
| BB | 1 | 1 |
| CC | 0 | 1 |
| DD | 0 | 0 |
| EE | 1 | 0 |
| FF | 1 | 0 |

Fitness.
   The accuracy of a reference model trained on the dataset using only the features present in the individual.
   Table C.

TABLE C

| Feature | Individual 1 | Individual 2 |
|---|---|---|
| AA | 1 | 1 |
| BB | 1 | 1 |
| CC | 0 | 1 |
| DD | 0 | 0 |
| EE | 1 | 0 |
| FF | 1 | 0 |
| Fitness | 0.60 | 0.70 |

Mutation.
   Randomly add/remove features from the individual
   (implementation detail-preserving mean number of included features).
   See mutation from Individual 1 to Individual 3 in Table D.

TABLE D

| Feature | Individual 1 | Individual 3 |
|---|---|---|
| AA | 1 | 1 |
| BB | 1 | 1 |
| CC | 0 | 0 |
| DD | 0 | 1 |
| EE | 1 | 0 |
| FF | 1 | 1 |

Mating:
   Choosing 50% of features from each ‘parent’.
   Table E shows Individual 4 following mating of Individual 1 and Individual 2.

TABLE E

| Feature | Individual 1 | Individual 2 | Individual 4 |
|---|---|---|---|
| AA | 1 | 1 | 1 |
| BB | 1 | 1 | 1 |
| CC | 0 | 1 | 1 |
| DD | 0 | 0 | 0 |
| EE | 1 | 0 | 1 |
| FF | 1 | 0 | 1 |

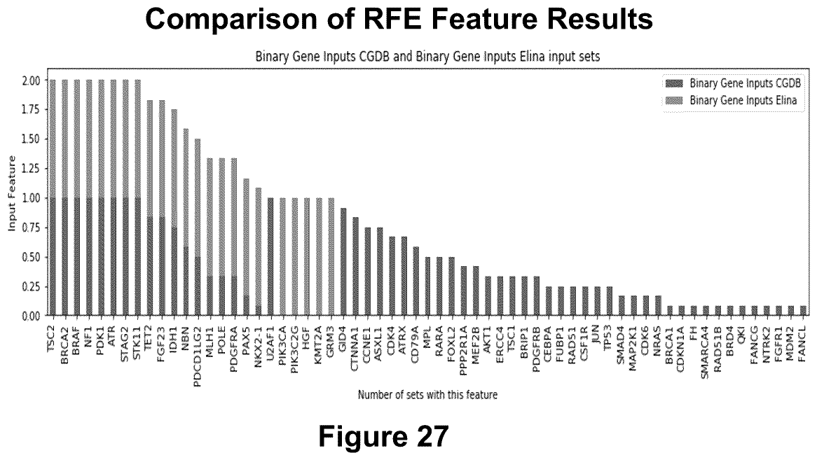

### Comparison to RFE Feature Results (FIG. 27):

Elina=RFE results;
CGDB=GA results.
Highly similar ‘core’ of important features.

### Points of Understandable Confusion

We use reference predicative models to evaluate the feature sets.
However, these are not tuned to maximize prediction, the models are meant to compare feature sets to each other.
   E.g., when we state that the accuracy is ˜60%, that should be interpreted as a measure of the features themselves, not the maximum performance of a trained model.
   The particular kinds of models we use currently for evaluating features are very simple linear models.
      This is to avoid overtraining on the huge potential set of features.
      Once a small subset of features is chosen, non-linear models have a chance of performing much better.

### Technical Details of Genetic Algorithm Settings

Three input dataset definitions.
   “Binary gene”—if a gene is mutated in any way, value is 1, else 0.
   “Loss or gain of function”-2 features per gene, one for gain-of-function mutations and another for loss-of-function mutations.
   “Hotspot granular”-common SV mutations are split out as additional features on top o the “loss or gain of function mutations”.
Binary features and binary target-only dura-response and inn-resistance patients used.
Genetic Algorithm Parameters:
   200 generations.
   Each generation is 1000 individuals.
   Fitness is defined as the 5-fold cross validated class-balanced accuracy of a Naïve Bayes (Bernoulli prior) model on the training set of patients.
   Individuals are chosen for mutation/mating probabilistically scaled by their fitness.
   Initial generation has 10% of features each on average.
   Mutation algorithm is adapted to preserve mean number of features (avoids growth of features).
   60% of each generation comes from mutation, 40% from mating.
Post-Processing (FIG. 16B).
   To improve confidence in robust result, GA is run 10 times from scratch.
   All individuals from all 10 runs are grouped together.
   The top 5% of all individuals over all 10 runs are clustered into 12 clusters via K-means.
   The features which appear in more than 50% of cluster members is defined as the “characteristic features” of the cluster and are the final outputs of the Genetic Algorithm.

### Genetic Algorithm.

Final outputs:
   12 sets of features for each of the 3 input definitions.
Evaluation:
   Any set which has performance on the held-out test set better than chance is kept.
   All 36 pass.
   Performance is ˜60% for all feature sets.

### Genetic Algorithm Outline

Basic idea is to search for sets of input features that make the best predictions of CPI resistance category.
   “Individuals” are sets of features (genes, mutations, or pathways); represented as a binary mask of the features.
   “Population” is a set of different individuals. “Fitness” is the per-individual prediction score for a model trained using only that individual's features.
Each generation:
   Mutate some individuals.
   Mate some individuals.
   Re-calculate the fitness of new individuals. \

### Genetic Algorithm Implementation Details

Predictive model chosen is a Random Forest (due to robustness to overfitting and simple structure).
Predictive score used is the “Log Loss” (or Cross Entropy).
   Often used in Neural Network training.
   Incorporates information about the probability of class assignment, not just class prediction.
   Handles multi-class predictions gracefully.
The first generation is randomly generated, which each feature having a 10% of being used to each individual.
For each generation:
   Combine the previous ‘parents’ (top 20% of all previous generations) and current generation.
   Rank each induvial according to its fitness.
   Make ⅗ population mutants by sampling from the fitness rankings (fitter individuals are more likely).
   Make ⅖ population crossovers by sampling from the fitness rankings (fitter individuals are more likely).

### Genetic Algorithm (GA) Example

Input dataset is binary gene mutation info. Table F.

TABLE F

| Patient ID | AA | AB | AC | AD | CPI |
|---|---|---|---|---|---|
| Pat1 | 0 | 0 | 0 | 1 | 0 |
| Pat2 | 0 | 1 | 1 | 1 | 1 |
| Pat3 | 1 | 0 | 0 | 1 | 2 |

GA individuals are randomly generated in the 1st generation.
   10% chance any single feature is included.
   Table G.

TABLE G

| Individual Name | AA | AB | AC | AD |
|---|---|---|---|---|
| A | 1 | 1 | 1 | 1 |
| B | 0 | 0 | 0 | 1 |
| C | 0 | 1 | 1 | 0 |

Individual fitnesses are calculated
   (The average cross validated log-loss over 5 fold is computed for Random Forest model predicting the CPI category using only the individual's features over all input date).
   Table H.

TABLE H

| Individual Name | AA | AB | AC | AD | Fitness |
|---|---|---|---|---|---|
| A | 1 | 1 | 1 | 1 | −2 |
| B | 0 | 0 | 0 | 1 | −1.5 |
| C | 0 | 1 | 1 | 0 | −1 |

New individuals are made by mutating previous individuals. Table I.

TABLE I

| | Individual Name | AA | AB | AC | AD | Fitness |
|---|---|---|---|---|---|---|
| Old | A | 1 | 1 | 1 | 1 | −2 |
| New | D | 1 | 1 | 0 | 1 | |

New individuals are made by mating previous individuals. Table J.

TABLE J

| | Individual Name | AA | AB | AC | AD | Fitness |
|---|---|---|---|---|---|---|
| Parent 1 | A | 1 | 1 | 1 | 1 | −2 |
| Parent 2 | B | 0 | 0 | 0 | 0 | −.15 |
| NEW Child | E | 0 | 1 | 0 | 1 | |

New individuals are evaluated. Table K.

TABLE K

| Individual Name | AA | AB | AC | AD | Fitness |
|---|---|---|---|---|---|
| D | 1 | 1 | 0 | 1 | −0.75 |
| E | 0 | 1 | 0 | 1 | −0.4 |

Cycle repeats for N generations.

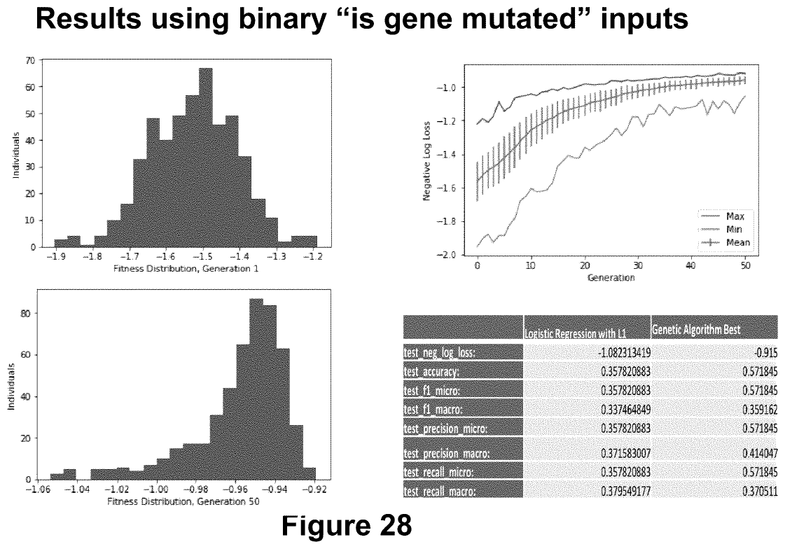

### Results Using Binary “is Gene Mutated” Inputs are Shown in FIG. 28.

### Information we can Get Out of the GA Results

When the model predictions are good (at least better than chance) for many individuals, we can compare the features used by those good individuals.
   If most individuals use a certain feature or set of features, they are likely important.
   If no individuals use a certain feature or set of features, they are likely unimportant.
   If there are individuals with two distinct sets of features that both perform well (and the features are not highly correlated) this could indicate separate gene networks affecting CPI resistance.
From these preliminary results as an example:
   Lists of genes/features that *always* appear in the top 20% of individuals:
   FAT3, KEAP1, NKX2-1, RBM10.

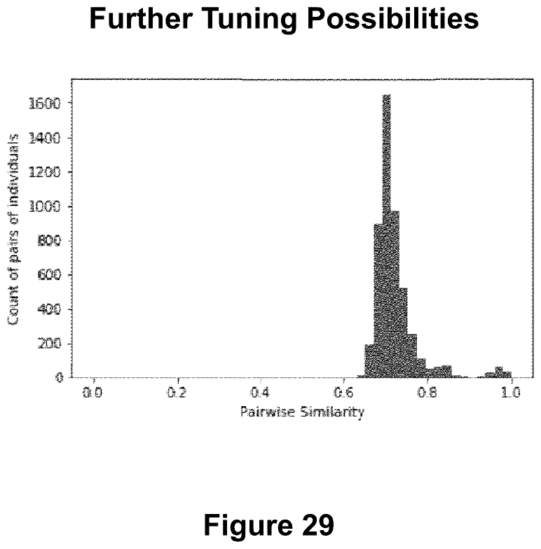

### Further Tuning Possibilities (FIG. 29)

The GA algorithm can be tuned to favor small sets of features.
   Maybe it's interesting to know if certain small subsets of features make models that are “good enough”.
The GA algorithm can be tuned to force high “diversity” among individuals.
   Maybe finding distinct sets of features that are both predictive may teach us about genetic networks.

### REFERENCES

A number of publications are cited above in order to more fully describe and disclose the invention and the state of the art to which the invention pertains. The entirety of each of these references is incorporated herein.
1. Rotow, J. & Bivona, T. G. Understanding and targeting resistance mechanisms in NSCLC. Nature Reviews Cancer (2017) doi: 10.1038/nrc.2017.84.
2. Inamura, K. Lung cancer: understanding its molecular pathology and the 2015 wHO classification. Front. Oncol. (2017) doi: 10.3389/fonc.2017.00193.
3. Schadendorf, D. et al. Efficacy and safety outcomes in patients with advanced melanoma who discontinued treatment with nivolumab and ipilimumab because of adverse events: A pooled analysis of randomized phase II and III trials. J. Clin. Oncol. (2017) doi: 10.1200/JCO.2017.73.2289.
4. Fehrenbacher, L. et al. Updated Efficacy Analysis Including Secondary Population Results for OAK: A Randomized Phase III Study of Atezolizumab versus Docetaxel in Patients with Previously Treated Advanced Non-Small Cell Lung Cancer. J. Thorac. Oncol. (2018) doi: 10.1016/j.jtho. 2018.04.039.
5. Bernard-Tessier, A. et al. Outcomes of long-term responders to anti-programmed death 1 and anti-programmed death ligand 1 when being rechallenged with the same anti-programmed death 1 and anti-programmed death ligand 1 at progression. Eur. J. Cancer (2018) doi: 10.1016/j.ejca.2018.06.005.
6. Mazein, A., Watterson, S., Hsieh, W. Y., Griffiths, W. J. & Ghazal, P. A comprehensive machine-readable view of the mammalian cholesterol biosynthesis pathway. Biochem. Pharmacol. (2013) doi: 10.1016/j.bcp.2013.03.021.
7. Antonia, S. J. et al. Four-year survival with nivolumab in patients with previously treated advanced non-small-cell lung cancer: a pooled analysis. Lancet Oncol. (2019) doi: 10.1016/S1470-2045 (19) 30407-3.
8. Schoenfeld, A. J. & Hellmann, M. D. Acquired Resistance to Immune Checkpoint Inhibitors. Cancer Cell vol. 37 443-455 (2020).
9. Sharma, P., Hu-Lieskovan, S., Wargo, J. A. & Ribas, A. Primary, Adaptive, and Acquired Resistance to Cancer Immunotherapy. Cell (2017) doi: 10.1016/j.cell.2017.01.017.
10. Walsh, R. J. & Soo, R. A. Resistance to immune checkpoint inhibitors in non-small cell lung cancer: biomarkers and therapeutic strategies. Ther. Adv. Med. Oncol. 12, 1-22 (2020).
11. Lagos, G. G., Izar, B. & Rizvi, N. A. Beyond Tumor PD-L1: Emerging Genomic Biomarkers for Checkpoint Inhibitor Immunotherapy. Am. Soc. Clin. Oncol. Educ. B. (2020) doi: 10.1200/edbk 289967.
12. Kalbasi, A. & Ribas, A. Tumour-intrinsic resistance to immune checkpoint blockade. Nature Reviews Immunology vol. 20 25-39 (2020).
13. Jiang, P. et al. Signatures of T cell dysfunction and exclusion predict cancer immunotherapy response. Nat. Med. (2018) doi: 10.1038/s41591-018-0136-1.
14. Hugo, W. et al. Genomic and Transcriptomic Features of Response to Anti-PD-1 Therapy in Metastatic Melanoma. Cell (2016) doi: 10.1016/j.cell.2016.02.065.
15. Auslander, N. et al. Robust prediction of response to immune checkpoint blockade therapy in metastatic melanoma. Nat. Med. (2018) doi: 10.1038/s41591-018-0157-9.
16. Anagnostou, V. et al. Integrative Tumor and Immune Cell Multi-omic Analyses Predict Response to Immune Checkpoint Blockade in Melanoma. Cell Reports Med. 1, (2020).
17. Singal, G. et al. Association of Patient Characteristics and Tumor Genomics With Clinical Outcomes Among Patients With Non-Small Cell Lung Cancer Using a Clinicogenomic Database. JAMA (2019) doi: 10.1001/jama.2019.3241.
18. Frampton, G. M. et al. Development and validation of a clinical cancer genomic profiling test based on massively parallel DNA sequencing. Nat. Biotechnol. (2013) doi: 10.1038/nbt.2696.
19. Kugel, C. H. et al. Age correlates with response to anti-PD1, reflecting age-related differences in intratumoral effector and regulatory T-cell populations. Clin. Cancer Res. (2018) doi: 10.1158/1078-0432.CCR-18-1116.
20. Rizvi, N. A. et al. Mutational landscape determines sensitivity to PD-1 blockade in non-small cell lung cancer. Science (80-.). (2015) doi: 10.1126/science.aaa1348.
21. Norum, J. & Nieder, C. Tobacco smoking and cessation and PD-L1 inhibitors in non-small cell lung cancer (NSCLC): A review of the literature. ESMO Open (2018) doi: 10.1136/esmoopen-2018-000406.
22. Xu, J. et al. Heterogeneity of Li-Fraumeni Syndrome links to unequal gain-of-function effects of p53 mutations. Sci. Rep. (2014) doi: 10.1038/srep04223.
23. Brosh, R. & Rotter, V. When mutants gain new powers: News from the mutant p53 field. Nature Reviews Cancer (2009) doi: 10.1038/nrc2693.
24. Summers, M. G. et al. BRAF and NRAS locus-specific variants have different outcomes on survival to colorectal cancer. Clin. Cancer Res. (2017) doi: 10.1158/1078-0432.CCR-16-1541.
25. De Roock, W. et al. Association of KRAS p. G13D mutation with outcome in patients with chemotherapy-refractory metastatic colorectal cancer treated with cetuximab. JAMA—J. Am. Med. Assoc. (2010) doi: 10.1001/jama.2010.1535.
26. Kadosh, E. et al. The gut microbiome switches mutant p53 from tumour-suppressive to oncogenic. Nature (2020) doi: 10.1038/s41586-020-2541-0.
27. Vogelstein, B. et al. Cancer genome landscapes. Science (2013) doi: 10.1126/science. 1235122.
28. Skoulidis, F. et al. STK11/LKB1 mutations and PD-1 inhibitor resistance in KRAS-mutant lung adenocarcinoma. Cancer Discov. (2018) doi: 10.1158/2159-8290.CD-18-0099.
29. Skoulidis, F. et al. Co-occurring genomic alterations define major subsets of KRAS-mutant lung adenocarcinoma with distinct biology, immune profiles, and therapeutic vulnerabilities. Cancer Discov. (2015) doi: 10.1158/2159-8290.CD-14-1236.
30. Blumenthal, G. M. et al. Overall response rate, progression-free survival, and overall survival with targeted and standard therapies in advanced non-small-cell lung cancer: US Food and Drug Administration trial-level and patient-level analyses. J. Clin. Oncol. (2015) doi: 10.1200/JCO.2014.59.0489.
31. Solomon, B. J. et al. Correlation between overall response rate and progression-free survival/overall survival in comparative trials involving targeted therapies in molecularly enriched populations. J. Clin. Oncol. (2020) doi: 10.1200/jco.2020.38.15 suppl.3588.
32. Papillon-Cavanagh, S., Doshi, P., Dobrin, R., Szustakowski, J. & Walsh, A. M. STK11 and KEAP1 mutations as prognostic biomarkers in an observational real-world lung adenocarcinoma cohort. ESMO Open 5, e000706 (2020).
33. Rish, I. An empirical study of the naive Bayes classifier. IJCAI 2001 Work. Empir, methods Artif. Intell. (2001) doi: 10.1039/b104835j.
34. Van Allen, E. M. et al. Genomic correlates of response to CTLA-4 blockade in metastatic melanoma. Science (80-.). (2015) doi: 10.1126/science.aad0095.
35. Miao, D. et al. Genomic correlates of response to immune checkpoint blockade in microsatellite-stable solid tumors. Nat. Genet. (2018) doi: 10.1038/s41588-018-0200-2.
36. Ng, A. Y. & Jordan, M. I. On discriminative vs. Generative classifiers: A comparison of logistic regression and naive bayes. in Advances in Neural Information Processing Systems (2002).
37. Hill, A. et al. Benchmarking network algorithms for contextualizing genes of interest. PLOS Comput. Biol. (2019) doi: 10.1371/journal.pcbi. 1007403.
38. O'Donnell, J. S., Long, G. V., Scolyer, R. A., Teng, M. W. L. & Smyth, M. J. Resistance to PD1/PDL1 checkpoint inhibition. Cancer Treatment Reviews (2017) doi: 10.1016/j.ctrv.2016.11.007.
39. Jackson, C. M., Choi, J. & Lim, M. Mechanisms of immunotherapy resistance: lessons from glioblastoma. Nature Immunology (2019) doi: 10.1038/s41590-019-0433-y.
40. Baugh, E. H., Ke, H., Levine, A. J., Bonneau, R. A. & Chan, C. S. Why are there hotspot mutations in the TP53 gene in human cancers? Cell Death and Differentiation (2018) doi: 10.1038/cdd.2017.180.
41. Levine, A. J. p53: 800 million years of evolution and 40 years of discovery. Nature Reviews Cancer www.nature.com/nrc doi: 10.1038/s41568-020-0262-1.
42. Bargonetti, J. & Prives, C. Gain-of-function mutant p53: History and speculation. Journal of Molecular Cell Biology (2019) doi: 10.1093/jmcb/mjz067.
43. Cormedi, M. C. V., Van Allen, E. M. & Colli, L. M. Predicting immunotherapy response through genomics. Current Opinion in Genetics and Development vol. 66 1-9 (2021).
44. Liu, H. J. et al. TSC2-deficient tumors have evidence of T cell exhaustion and respond to anti-PD-1/anti-CTLA-4 immunotherapy. JCI insight (2018) doi: 10.1172/jci.insight.98674.
45. Torrejon, D. Y. et al. Overcoming Genetically Based Resistance Mechanisms to PD-1 Blockade. Cancer Discov. (2020) doi: 10.1158/2159-8290.CD-19-1409.
46. Liang, Y. et al. Targeting IFNα to tumor by anti-PD-L1 creates feedforward antitumor responses to overcome checkpoint blockade resistance. Nat. Commun. (2018) doi: 10.1038/s41467-018-06890-y.
47. Reislander, T., Groelly, F. J. & Tarsounas, M. DNA Damage and Cancer Immunotherapy: A STING in the Tale. Molecular Cell (2020) doi: 10.1016/j.molcel.2020.07.026.
48. Gao, S. P. et al. Mutations in the EGFR kinase domain mediate STAT3 activation via IL-6 production in human lung adenocarcinomas. J. Clin. Invest. 117, 3846-3856 (2007).
49. Jiang, L. et al. Continuous targeted kinase inhibitors treatment induces upregulation of PD-L1 in resistant NSCLC. Sci. Rep. (2019) doi: 10.1038/s41598-018-38068-3.
50. Liu, H., Shen, J. & Lu, K. IL-6 and PD-L1 blockade combination inhibits hepatocellular carcinoma cancer development in mouse model. Biochem. Biophys. Res. Commun. 486, 239-244 (2017).
51. Bialkowski, L. et al. Immune checkpoint blockade combined with IL-6 and TGF-β inhibition improves the therapeutic outcome of mRNA-based immunotherapy. Int. J. Cancer 143, 686-698 (2018).
52. Keegan, A. et al. Plasma IL-6 changes correlate to PD-1 inhibitor responses in NSCLC. J. Immunother. Cancer (2020) doi: 10.1136/jitc-2020-000678.
53. Garbers, C., Heink, S., Korn, T. & Rose-John, S. Interleukin-6: Designing specific therapeutics for a complex cytokine. Nature Reviews Drug Discovery (2018) doi: 10.1038/nrd.2018.45.
54. Takeuchi, T. et al. Considering new lessons about the use of IL-6 inhibitors in arthritis. Considerations Med. (2018) doi: 10.1136/conmed-2018-000002.
55. Yuen, K. C. et al. Abstract 2676: Associations of peripheral biomarkers to outcomes to anti-PD-L1 immune checkpoint blockade in metastatic urothelial cancer. in (2019). doi: 10.1158/1538-7445.am2019-2676.
56. çelik, A. et al. Angiogenic and Immune-Related Biomarkers and Outcomes Following Axitinib/Pembrolizumab Treatment in Patients with Advanced Renal Cell Carcinoma. J. Mater. Process. Technol. 1, 1-8 (2018).
57. Zhao, J. et al. Immune and genomic correlates of response to anti-PD-1 immunotherapy in glioblastoma. Nat. Med. (2019) doi: 10.1038/s41591-019-0349-y.
58. Luke, J. J., Bao, R., Sweis, R. F., Spranger, S. & Gajewski, T. F. WNT/b-catenin pathway activation correlates with immune exclusion across human cancers. Clin. Cancer Res. (2019) doi: 10.1158/1078-0432.CCR-18-1942.
59. Öhlund, D. et al. Distinct populations of inflammatory fibroblasts and myofibroblasts in pancreatic cancer. J. Exp. Med. (2017) doi: 10.1084/jem.20162024.
60. Evans, E. K. et al. C A N C E R A precision therapy against cancers driven by KIT/PDGFRA mutations. www.cellsignal.com (2017).
61. Kim, G. & Ko, Y. T. Small molecule tyrosine kinase inhibitors in glioblastoma. Archives of Pharmacal Research vol. 43 385-394 (2020).
62. Tassell, V. et al. Preliminary biomarker analysis of sitravatinib in combination with nivolumab in NSCLC patients progressing on prior checkpoint inhibitor. J. Immunother. Cancer (2018).
63. Brose, M. S. et al. A phase Ib/II trial of lenvatinib plus pembrolizumab in non-small cell lung cancer. J. Clin. Oncol. (2019) doi: 10.1200/jco.2019.37.8 suppl.16.
64. Du, W., Huang, H., Sorrelle, N. & Brekken, R. A. Sitravatinib potentiates immune checkpoint blockade in refractory cancer models. JCI insight (2018) doi: 10.1172/jci.insight.124184.

1. A computer-implemented method of predicting whether a patient is likely to display resistance to a predetermined treatment, the computer-implemented method comprising:
receiving a genetic feature profile comprising a binary mask comprising a feature status of each of an identified set of genetic features;
applying an analytical model to the received genetic feature profile, wherein the analytical model has been trained by:
(i) determining one or more sets of genetic features, wherein determining the one or more sets of genetic features comprises:
(a) receiving patient data comprising, for each of a plurality of patients: an indication of whether that patient is resistant to the predetermined treatment, and a genetic feature profile comprising a binary mask, the binary mask comprising:
for each of one or more genes, an indication of whether there is a mutation at any point in that gene; and
for each mutation, at least one of: (1) an indication of whether the mutation is a gain-of-function mutation or a loss-of-function mutation, or (2) an indication of the position of that mutation within the gene in which it is located, the indication comprising, for each of a plurality of hotspot locations within a given gene, an indication of whether the mutation is present at that hotspot;
(b) using a genetic algorithm to generate a plurality of generations of individuals, wherein each individual comprises a subset of the predetermined plurality of genetic features, each generation of individuals generated based, at least in part, on a plurality of fitness scores, each fitness score corresponding to a respective individual in the previous generation, and parameterizing a predictive accuracy of the set of genetic features, each fitness score being calculated based at least in part on the patient data;
(c) repeating step (b) until it has been performed N times;
(d) from the plurality of individuals generated in steps (b) and (c), selecting a subset of the individuals based on their fitness scores;
(e) clustering the selected subset of individuals to generate a plurality of clusters of individuals, based on the similarity of their respective subsets of features; and
(f) from each cluster, identifying a respective characteristic genetic feature set based on the frequency with which genetic features appear in individuals in that cluster; and
(ii) training the analytical model using training data related to the one or more identified sets of genetic features, to generate the trained analytical model; and
outputting a result indicative of whether the patient is likely to display resistance to the predetermined treatment.

2. The computer-implemented method of claim 1, wherein:
the received genetic feature profile comprises a binary mask, the binary mask comprising:
for each of one or more genes, an indication of whether there is a mutation at any point in that gene; and
for each mutation, at least one of:
(1) an indication of whether the mutation is a gain-of-function mutation or a loss-of-function mutation; or
(2) an indication of the position of that mutation within the gene in which it is located, the indication comprising, for each of a plurality of hotspot locations within a given gene, an indication of whether the mutation is present at that hotspot.

3. The computer-implemented method of claim 1, wherein:
step (f) comprises:
for each cluster of individuals, identifying the one or more genetic features which occur in more than a threshold proportion of individuals within that cluster, those features forming the respective characteristic genetic feature set for that cluster; and
selecting one or more of the characteristic genetic feature sets of the respective plurality of clusters as the one or more genetic feature sets to predict the resistance to the predetermined treatment.

4. The computer-implemented method of claim 1, wherein:
step (e) comprises applying a k-means clustering algorithm on the selected subsets of individuals.

5. The computer-implemented method of claim 1, wherein:
N is no less than 10; and
the plurality of clusters comprises at least N clusters.

6. The computer-implemented method of claim 1, wherein:
the patient data comprises a first subset of patient data and a second subset of patient data;
each fitness score is calculated based at least in part on the first subset of patient data, and not on the second subset of patient data; and
step (f) comprises:
for each identified characteristic genetic feature set, calculating a fitness score parameterizing the predictive accuracy of the characteristic genetic feature set, based at least in part on the first subset of patient data, and not the second subset of patient data; and
selecting the one or more characteristic genetic feature sets having the highest associated fitness score as the one or more genetic feature sets which best predict the likelihood that the patient is resistant to the predetermined treatment.

7. The computer-implemented method of claim 6, wherein:
during step (b), the fitness score is a cross-validation accuracy score of a naïve Bayes model, with a Bernoulli prior, on a training set which comprises a first subset of the patient data.

8. The computer-implemented method of claim 1, wherein:
using the genetic algorithm to generate the plurality of generations of individuals comprises:
(i) generating a plurality of first generation G₁ individuals, and for each first generation individual, calculating a fitness score;
(ii) generating a plurality of second generation G₂ individuals, the subset of genetic features of each respective second generation individual being based on the subset of genetic features of at least one first generation individual and, for each second generation individual, calculating a fitness score; and
(iii) generating a subsequent generation Gᵢ of individuals, the subset of genetic features of each respective individual in the subsequent generation of individuals being generated based on the subset of genetic features of at least one individual in the previous generation Gᵢ₋₁ of individuals, and, for each individual in the subsequent generation of individuals, calculating a fitness score; and
(iv) iteratively repeating step (iii) until the plurality of generations of individual has been generated.

9. The computer-implemented method of claim 8, wherein:
generating the plurality of second generation individuals comprises, for each of one or more second generation individuals:
sampling the plurality of first generation individuals to select a candidate individual, wherein the probability of a given first generation individual being sampled is based on the respective fitness score of that individual; and
mutating the subset of genetic features of the candidate individual to generate a mutated subset of genetic features, thereby generating a second generation individual having as their subset of genetic features the mutated subset of genetic features.

10. The computer-implemented method of claim 8, wherein:
generating the plurality of second generation individuals comprises, for each of one or more second generation individuals:
sampling the plurality of first generation individuals to select a first parent individual a second parent individual, wherein the probability of a given first generation individual being selected is based on the respective fitness score of that individual; and
mating the first parent individual and the second parent individual from the first generation, thereby generating a second generation individual whose subset of genetic features is based on the respective subsets of genetic features of the first parent individual and the second parent individual.

11. A computer-implemented method of generating an analytical model for predicting the presence or absence of a particular phenotypic characteristic, the computer-implemented invention comprising:
determining one or more sets of genetic features, wherein determining the one or more sets of genetic features comprises:
(a) receiving patient data comprising, for each of a plurality of patients: an indication of whether that patient is resistant to the predetermined treatment, and a genetic feature profile comprising a binary mask, the binary mask comprising:
for each of one or more genes, an indication of whether there is a mutation at any point in that gene; and
for each mutation, at least one of: (1) an indication of whether the mutation is a gain-of-function mutation or a loss-of-function mutation, or (2) an indication of the position of that mutation within the gene in which it is located, the indication comprising, for each of a plurality of hotspot locations within a given gene, an indication of whether the mutation is present at that hotspot;
(b) using a genetic algorithm to generate a plurality of generations of individuals, wherein each individual comprises a subset of the predetermined plurality of genetic features, each generation of individuals generated based, at least in part, on a plurality of fitness scores, each fitness score corresponding to a respective individual in the previous generation, and parameterizing a predictive accuracy of the set of genetic features, each fitness score being calculated based at least in part on the patient data;
(c) repeating step (b) until it has been performed N times;
(d) from the plurality of individuals generated in steps (b) and (c), selecting a subset of the individuals based on their fitness scores;
(e) clustering the selected subset of individuals to generate a plurality of clusters of individuals, based on the similarity of their respective subsets of features; and
(f) from each cluster, identifying a respective characteristic genetic feature set based on the frequency with which genetic features appear in individuals in that cluster; and
training an analytical model using training data relating to the one or more sets of genetic features to generate a trained analytical model.

12. A system comprising a processor configured to execute the computer-implemented method of claim 1.

13. The computer-implemented method of claim 2, wherein:
step (f) comprises:
for each cluster of individuals, identifying the one or more genetic features which occur in more than a threshold proportion of individuals within that cluster, those features forming the respective characteristic genetic feature set for that cluster; and
selecting one or more of the characteristic genetic feature sets of the respective plurality of clusters as the one or more genetic feature sets to predict the resistance to the predetermined treatment.

14. The computer-implemented method of claim 2, wherein:
the patient data comprises a first subset of patient data and a second subset of patient data;
each fitness score is calculated based at least in part on the first subset of patient data, and not on the second subset of patient data; and
step (f) comprises:
for each identified characteristic genetic feature set, calculating a fitness score parameterizing the predictive accuracy of the characteristic genetic feature set, based at least in part on the first subset of patient data, and not the second subset of patient data; and
selecting the one or more characteristic genetic feature sets having the highest associated fitness score as the one or more genetic feature sets which best predict the likelihood that the patient is resistant to the predetermined treatment.

15. The computer-implemented method of claim 2, wherein:
using the genetic algorithm to generate the plurality of generations of individuals comprises:
(i) generating a plurality of first generation G₁ individuals, and for each first generation individual, calculating a fitness score;
(ii) generating a plurality of second generation G₂ individuals, the subset of genetic features of each respective second generation individual being based on the subset of genetic features of at least one first generation individual and, for each second generation individual, calculating a fitness score; and
(iii) generating a subsequent generation Gᵢ of individuals, the subset of genetic features of each respective individual in the subsequent generation of individuals being generated based on the subset of genetic features of at least one individual in the previous generation Gᵢ₋₁ of individuals, and for each individual in the subsequent generation of individuals, calculating a fitness score; and
(iv) iteratively repeating step (iii) until the plurality of generations of individuals has been generated.

16. The computer-implemented method of claim 15, wherein:
generating the plurality of second generation individuals comprises, for each of one or more second generation individuals:
sampling the plurality of first generation individuals to select a candidate individual, wherein the probability of a given first generation individual being sampled is based on the respective fitness score of that individual; and
mutating the subset of genetic features of the candidate individual to generate a mutated subset of genetic features, thereby generating a second generation individual having as their subset of genetic features the mutated subset of genetic features.

17. The computer-implemented method of claim 15, wherein:
generating the plurality of second generation individuals comprises, for each of one or more second generation individuals:
sampling the plurality of first generation individuals to select a first parent individual a second parent individual, wherein the probability of a given first generation individual being selected is based on the respective fitness score of that individual; and
mating the first parent individual and the second parent individual from the first generation, thereby generating a second generation individual whose subset of genetic features is based on the respective subsets of genetic features of the first parent individual and the second parent individual.

18. A system comprising a processor configured to execute the computer-implemented method of claim 2.

19. A system comprising a processor configured to execute the computer-implemented method of claim 13.

20. A system comprising a processor configured to execute the computer-implemented method of claim 14.
